(19) United States
(12) Patent Application Publication
(10) Pub. No.: US 2025/0368637 A1
(43) Pub. Date: Dec. 4, 2025

(54) SOLID-STATE FORMS OF N-(2-(4-CYANOTHIAZOLIDIN-3-YL)-2-OXOETHYL)-6-MORPHOLINOQUINOLINE-4-CARBOXAMIDE

(71) Applicant: AstraZeneca AB, Södertälje (SE)

(72) Inventors: Anna ARDANÉ, Gothenburg (GB); Staffan KARLSSON, Gothenburg (SE); Yvonne SAWYER, Macclesfield (GB)

(21) Appl. No.: 18/871,230

(22) PCT Filed: Jun. 20, 2023
(86) PCT No.: PCT/EP2023/066559

§ 371 Date: Dec. 3, 2024

Related U.S. Application Data
(60) Provisional application No. 63/366,696, filed on Jun. 21, 2022.

(51) Int. Cl. C07D 417/12; A61K 31/5377

(52) U.S. Cl. CPC C07D 417/12; A61K 31/5377

(57) ABSTRACT

Solid-state forms of N-(2-(4-cyanothiazolidin-3-yl)-2-oxoethyl)-6-morpholinoquinoline-4-carboxamide; corresponding pharmaceutical compositions; uses to treat or prevent Prolyl endopeptidase fibroblast activation protein (FAP)-mediated conditions; kits; and methods of preparation.

### CROSS-REFERENCE TO RELATED APPLICATIONS

This application claims the benefit under 35 U.S.C. § 119 (e) of U.S. Provisional Application No. 63/366,696, filed Jun. 21, 2022. The above-listed application is incorporated by reference in its entirety for all purposes.

### FIELD

The present disclosure relates generally to solid-state forms of N-(2-(4-cyanothiazolidin-3-yl)-2-oxoethyl)-6-morpholinoquinoline-4-carboxamide, including crystalline forms of (R)—N-(2-(4-cyanothiazolidin-3-yl)-2-oxoethyl)-6-morpholinoquinoline-4-carboxamide. The present disclosure further relates to pharmaceutical compositions comprising a crystalline form of (R)—N-(2-(4-cyanothiazolidin-3-yl)-2-oxoethyl)-6-morpholinoquinoline-4-carboxamide; use of a pharmaceutical composition comprising a crystalline form of (R)—N-(2-(4-cyanothiazolidin-3-yl)-2-oxoethyl)-6-morpholinoquinoline-4-carboxamide to treat or prevent Prolyl endopeptidase fibroblast activation protein (FAP)-mediated conditions; kits comprising a pharmaceutical composition comprising a crystalline form of (R)—N-(2-(4-cyanothiazolidin-3-yl)-2-oxoethyl)-6-morpholinoquinoline-4-carboxamide; and methods for preparing crystalline forms of (R)—N-(2-(4-cyanothiazolidin-3-yl)-2-oxoethyl)-6-morpholinoquinoline-4-carboxamide.

### BACKGROUND

FAP, a type II transmembrane serine protease, is expressed by fibroblast like cells involved in tissue remodeling and healing. In the context of non-alcoholic steatohepatitis (NASH), FAP is upregulated on the cell surface of activated hepatic stellate cells involved in the fibrosis formation (Hepatology 1999, 29, 1768), a major aspect of NASH that predicts disease outcome (Gastroenterology 2020, 158, 1611). FAP also can be present as a shedded plasma protease. Increased levels of circulating FAP are associated with NASH disease severity (Diabetes Res Clin Pract 2015, 108, 466).

FAP has a consensus cleavage motif after Gly-Pro and exhibits both endopeptidase and exopeptidase activity. Known enzymatic activities include cleavage of collagens (Hepatology 1999, 29, 1768), α2-antiplasmin (a2AP) (Blood 2004 103, 3783), and fibroblast growth factor 21 (FGF21) (Biochem J 2016, 473, 605). FAP activity at the cell surface of activated fibroblasts (including cleavage of collagens) generates a pro-fibrotic environment. FAP cleavage of a2AP gives a more efficient cross-linking of a2AP to fibrin and results in reduced fibrin clearance. FAP cleavage of FGF21 inactivates FGF21 metabolic effects (Biochem J 2016, 473, 605). All these activities are associated with a worsening of NASH disease and inhibiting FAP has the potential to treat NASH and other conditions by affecting multiple mechanisms.

Inhibition of FAP activity is a presently unexploited therapeutic approach for treating NASH and other diseases associated with such activity. No approved pharmacological agents that inhibit FAP activity generally, or that inhibit FAP activity specifically, are currently available. Accordingly, there is a need for FAP inhibitors, particularly FAP inhibitors that have pharmacologically appropriate selectivity and bioavailability and that possess physical properties suitable for manufacturing a drug substance and formulating a corresponding drug product.

The present disclosure addresses this large unmet need by providing solid-state forms of the FAP inhibitor, N-(2-(4-cyanothiazolidin-3-yl)-2-oxoethyl)-6-morpholinoquinoline-4-carboxamide, that are suitable for use in pharmaceutical compositions and methods for treating FAP-mediated conditions such as NASH.

### SUMMARY

In one aspect, the present disclosure provides solid-state forms of N-(2-(4-cyanothiazolidin-3-yl)-2-oxoethyl)-6-morpholinoquinoline-4-carboxamide.

In another aspect, the present disclosure provides crystalline forms of (R)—N-(2-(4-cyanothiazolidin-3-yl)-2-oxoethyl)-6-morpholinoquinoline-4-carboxamide.

In another aspect, the present disclosure provides crystalline (R)—N-(2-(4-cyanothiazolidin-3-yl)-2-oxoethyl)-6-morpholinoquinoline-4-carboxamide Form A.

In another aspect, the present disclosure provides crystalline (R)—N-(2-(4-cyanothiazolidin-3-yl)-2-oxoethyl)-6-morpholinoquinoline-4-carboxamide Form B.

In another aspect, the present disclosure provides crystalline forms of (R,S)—N-(2-(4-cyanothiazolidin-3-yl)-2-oxoethyl)-6-morpholinoquinoline-4-carboxamide.

In another aspect, the present disclosure provides crystalline (R,S)—N-(2-(4-cyanothiazolidin-3-yl)-2-oxoethyl)-6-morpholinoquinoline-4-carboxamide Type 2.

In another aspect, the present disclosure provides pharmaceutical compositions comprising a crystalline form of N-(2-(4-cyanothiazolidin-3-yl)-2-oxoethyl)-6-morpholino-quinoline-4-carboxamide, and one or more pharmaceutically acceptable excipients.

In another aspect, the present disclosure provides methods of treating or preventing a Prolyl endopeptidase fibroblast activation protein (FAP)-mediated condition by administering to a subject in need thereof a pharmaceutical composition comprising a therapeutically effective amount of a crystalline form of N-(2-(4-cyanothiazolidin-3-yl)-2-oxoethyl)-6-morpholino-quinoline-4-carboxamide.

In another aspect, the present disclosure provides use of a pharmaceutical composition comprising a crystalline form of N-(2-(4-cyanothiazolidin-3-yl)-2-oxoethyl)-6-morpholino-quinoline-4-carboxamide for treating or preventing a Prolyl endopeptidase fibroblast activation protein (FAP)-mediated condition.

In another aspect, the present disclosure provides use of a crystalline form of N-(2-(4-cyanothiazolidin-3-yl)-2-oxoethyl)-6-morpholinoquinoline-4-carboxamide for the manufacture of medicaments for treating or preventing a Prolyl endopeptidase fibroblast activation protein (FAP)-mediated condition.

In another aspect, the present disclosure provides kits comprising a pharmaceutical composition comprising a crystalline form of N-(2-(4-cyanothiazolidin-3-yl)-2-oxoethyl)-6-morpholinoquinoline-4-carboxamide.

In another aspect, the present disclosure provides methods for preparing crystalline forms of N-(2-(4-cyanothiazolidin-3-yl)-2-oxoethyl)-6-morpholinoquinoline-4-carboxamide.

### BRIEF DESCRIPTION OF THE DRAWINGS

FIG. 1 is a representative transmission powder X-ray diffraction pattern for crystalline (R)—N-(2-(4-cyanothiazolidin-3-yl)-2-oxoethyl)-6-morpholinoquinoline-4-carboxamide Form A.

FIG. 2 is a representative solid-state ¹³C NMR spectrum for a sample of crystalline (R)—N-(2-(4-cyanothiazolidin-3-yl)-2-oxoethyl)-6-morpholinoquinoline-4-carboxamide that is approximately 95 weight % Form A and 5 weight % Form B.

FIG. 3 is a representative solid-state ¹³C NMR spectrum for a sample of crystalline (R)—N-(2-(4-cyanothiazolidin-3-yl)-2-oxoethyl)-6-morpholinoquinoline-4-carboxamide that is approximately 30 weight % Form A and 70 weight % Form B.

FIG. 4 is a representative solid-state ¹³C NMR spectrum for a sample of crystalline (R)—N-(2-(4-cyanothiazolidin-3-yl)-2-oxoethyl)-6-morpholinoquinoline-4-carboxamide that is Form B with a minor amount of Form A.

FIG. 5 is a comparison of the solid-state ¹³C NMR spectra for crystalline (R)—N-(2-(4-cyanothiazolidin-3-yl)-2-oxoethyl)-6-morpholinoquinoline-4-carboxamide Form A and Form B based on the spectra shown in FIGS. 2, 3, and 4.

FIG. 6 is a representative differential scanning calorimetry curve for crystalline (R)—N-(2-(4-cyanothiazolidin-3-yl)-2-oxoethyl)-6-morpholinoquinoline-4-carboxamide Form A.

FIG. 7 is a representative thermogravimetric analysis thermogram for crystalline (R)—N-(2-(4-cyanothiazolidin-3-yl)-2-oxoethyl)-6-morpholinoquinoline-4-carboxamide Form A.

FIG. 8 is a representative gravimetric vapor sorption plot for crystalline (R)—N-(2-(4-cyanothiazolidin-3-yl)-2-oxoethyl)-6-morpholinoquinoline-4-carboxamide Form A.

FIG. 9 is a representative transmission powder X-ray diffraction pattern for crystalline (R)—N-(2-(4-cyanothiazolidin-3-yl)-2-oxoethyl)-6-morpholinoquinoline-4-carboxamide Form B.

FIG. 10 is a comparison of the transmission powder X-ray diffraction patterns for crystalline (R)—N-(2-(4-cyanothiazolidin-3-yl)-2-oxoethyl)-6-morpholinoquinoline-4-carboxamide Form A and Form B based on the diffractograms shown in FIGS. 1 and 9. The Form A diffractogram is at the top and the Form B diffractogram is at the bottom of FIG. 10.

FIG. 11 is a comparison of the transmission powder X-ray diffraction patterns for crystalline (R)—N-(2-(4-cyanothiazolidin-3-yl)-2-oxoethyl)-6-morpholinoquinoline-4-carboxamide Form A and Form B based on the diffractograms shown in FIGS. 1 and 9 for region encompassing the distinctive Form B peaks. The Form A diffractogram is at the top and the Form B diffractogram is at the bottom of FIG. 11.

FIG. 12 is a representative differential scanning calorimetry curve for crystalline (R)—N-(2-(4-cyanothiazolidin-3-yl)-2-oxoethyl)-6-morpholinoquinoline-4-carboxamide Form B.

FIG. 13 is a representative thermogravimetric analysis thermogram for crystalline (R)—N-(2-(4-cyanothiazolidin-3-yl)-2-oxoethyl)-6-morpholinoquinoline-4-carboxamide Form B.

FIG. 14 is a representative gravimetric vapor sorption plot for crystalline (R)—N-(2-(4-cyanothiazolidin-3-yl)-2-oxoethyl)-6-morpholinoquinoline-4-carboxamide Form B.

FIG. 15 is a representative reflection powder X-ray diffraction pattern for crystalline (R,S)—N-(2-(4-cyanothiazolidin-3-yl)-2-oxoethyl)-6-morpholinoquinoline-4-carboxamide Type 2.

FIG. 16 is a representative differential scanning calorimetry curve for crystalline (R,S)—N-(2-(4-cyanothiazolidin-3-yl)-2-oxoethyl)-6-morpholinoquinoline-4-carboxamide Type 2.

FIG. 17 is a representative gravimetric vapor sorption plot for crystalline (R,S)—N-(2-(4-cyanothiazolidin-3-yl)-2-oxoethyl)-6-morpholinoquinoline-4-carboxamide Type 2.

FIG. 18 is a representative reflection powder X-ray diffraction pattern for amorphous (R)—N-(2-(4-cyanothiazolidin-3-yl)-2-oxoethyl)-6-morpholinoquinoline-4-carboxamide.

FIG. 19 is a representative differential scanning calorimetry curve for amorphous (R)—N-(2-(4-cyanothiazolidin-3-yl)-2-oxoethyl)-6-morpholinoquinoline-4-carboxamide.

FIG. 20 is a representative gravimetric vapor sorption plot for amorphous (R)—N-(2-(4-cyanothiazolidin-3-yl)-2-oxoethyl)-6-morpholinoquinoline-4-carboxamide.

FIG. 21 shows representative solubility curves for (R)—N-(2-(4-cyanothiazolidin-3-yl)-2-oxoethyl)-6-morpholinoquinoline-4-carboxamide Form B in acetonitrile and methanol.

### DETAILED DESCRIPTION

Many embodiments are detailed throughout the specification and will be apparent to a reader skilled in the art. Such embodiments are provided by way of example only and are not intended to otherwise limit the scope of the invention. Various alternatives to the described embodiments may be employed in practicing the invention.

### I. Definitions

With respect to the embodiments disclosed in this specification, the following terms have the meanings set forth below:

Reference to “a” or “an” means “one or more.” Throughout, the plural and singular should be treated as interchangeable, other than the indication of number.

When ranges are used herein to describe, for example, physical or chemical properties, all combinations and subcombinations of ranges and specific embodiments therein are intended to be included. Use of the term “about” or “approximately” when referring to a number or a numerical range means that the number or numerical range referred to is an approximation within experimental variability (or within statistical experimental error), and thus the number or numerical range may vary from, for example, between 1% and 15% of the stated number or numerical range.

Unless the context requires otherwise, the words “comprise” or “comprises” or “comprising” are used on the basis and clear understanding that they are to be interpreted inclusively, rather than exclusively, and that Applicant intends each of those words to be so interpreted in construing this patent, including the claims below.

The term “amorphous form” refers to a form of a compound that lacks long range crystalline order.

The terms “co-administration,” “co-administering,” “administered in combination with,” and “administering in combination with” as used herein, encompass administration of two or more agents to a subject so that both agents and/or their metabolites are present in the subject at the same time. Co-administration includes simultaneous administration in separate compositions, administration at different times in separate compositions, or administration in a composition in which two or more agents are present.

The term “crystalline form” is intended to include all crystalline forms of the compound, including, for example, polymorphs, pseudopolymorphs, solvates, hydrates, unsolvated polymorphs (including anhydrates), and conformational polymorphs, as well as mixtures thereof, unless a particular crystalline form is referred to.

The term “therapeutically effective amount” of a pharmacological agent is an amount that is sufficient to effect beneficial or desired results, including clinical results, and, as such, will depend upon the situation in which it is being administered. Where the pharmacological agent is being administered to treat liver disease, for example, a therapeutically effective amount of the agent is an amount of the agent that is sufficient, either alone or in combination with additional therapies, to provide an anti-liver disease effect in a subject as compared to the response obtained without administration of the agent.

The term “pharmaceutically acceptable” is used adjectivally in this specification to mean that the modified noun is appropriate for use as a pharmaceutical product or as a part of a pharmaceutical product. For example, the term “pharmaceutically carrier” or “pharmaceutically acceptable excipient” is intended to include any and all carriers or excipients that are suitable for use in mammals, particularly humans.

The terms “reflection” or “reflection mode,” when used in conjunction with powder X-ray diffraction, refers to the reflection (also known as Bragg-Brentano) sampling mode.

The term “preventing” is readily understood by an ordinarily skilled physician and, with respect to treatment of a particular condition, can include is intended to have its normal meaning and includes primary prophylaxis to prevent the development of the condition and secondary prophylaxis whereby the condition has already developed and the patient is temporarily or permanently protected against exacerbation or worsening of the disease or the development of new symptoms associated with the condition.

The term “solvate” refers to a crystalline phase of a compound in physical association with one or more molecules of a solvent. The crystalline phase of a compound in physical association with one or more molecules of water is referred to as a “hydrate.”

The terms “transmission” or “transmission mode,” when used in conjunction with powder X-ray diffraction, refers to the transmission (also known as Debye-Scherrer) sampling mode.

The term “treating” is readily understood by an ordinarily skilled physician and, with respect to treatment of a particular condition, can include (1) diminishing the extent or cause of the condition being treated, and/or (2) alleviating or ameliorating one or more symptoms associated with that condition. Treatment of liver disease, for example, can include stabilizing (i.e., not worsening), delaying, or slowing the spread or progression of the liver disease: prolonging survival as compared to expected survival if not receiving treatment; and/or otherwise ameliorating or palliating the severity of the liver disease, in whole or in part.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the(S)-isomeric form is 80%. The enantiomeric purity of a compound can be determined in a number of ways, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts: or preferred enantiomers can be prepared by asymmetric syntheses. See, for example, Jacques, et al., Enantiomers, Racemates and Resolutions, Wiley Interscience, New York, 1981: Eliel, Stereochemistry of Carbon Compounds, McGraw-Hill, NY, 1962; and Eliel and Wilen, Stereochemistry of Organic Compounds, Wiley-Interscience, New York, 1994.

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (R)-enantiomer, means a preparation of the compound having greater than 50% by weight of the (R)-enantiomer relative to the (S)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

II. Crystalline Forms of N-(2-(4-Cyanothiazolidin-3-yl)-2-oxoethyl)-6-morpholinoquinoline-4-carboxamide

A compound that is an active pharmaceutical ingredient in a drug product potentially can exist in different solid-state forms exhibiting different physical properties. These physical property differences can impact the manufacturing and formulation of the drug product. Such physical properties can include, but are not limited to: (1) packing properties such as molar volume, density, and hygroscopicity, (2) thermodynamic properties such as melting temperature, vapor pressure, and solubility, (3) kinetic properties such as dissolution rate and stability (including stability at ambient conditions, especially to moisture and under storage conditions), (4) surface properties such as surface area, wettability, interfacial tension, and shape, (5) mechanical properties such as hardness, tensile strength, compressibility, compactibility, handling, flow and blend; and (6) filtration properties. Accordingly, solid-state forms of a compound, particularly crystalline forms of the compound, that provide an improvement in one or more of these physical properties relative to other solid-state forms of the compound are desirable. The discovery of a new solid-state form of a pharmaceutically useful compound therefore provides a potential opportunity to improve the performance characteristics of the corresponding drug product and related manufacturing process.

The present disclosure provides solid-state forms of N-(2-(4-cyanothiazolidin-3-yl)-2-oxoethyl)-6-morpholinoquinoline-4-carboxamide. In one aspect, the solid-state form is a crystalline form. Each crystalline form described in the present specification possesses one or more of the above-described advantageous properties relative to one or more of the other solid-state forms of the compound. In another aspect, the solid-state form is a crystalline anhydrate. In further aspects, the crystalline form is substantially pure. As used in the present specification, the term “substantially pure” means that the crystalline form of the compound comprises at least about 90 weight % of the desired crystalline form relative to any other solid-state form of the compound. In one aspect, the crystalline form of the compound comprises at least about 95 weight % of the desired crystalline form. In another aspect, the crystalline form of the compound comprises at least about 96 weight % of the desired crystalline form. In another aspect, the crystalline form of the compound comprises at least about 97 weight % of the desired crystalline form. In another aspect, the crystalline form of the compound comprises at least about 98 weight % of the desired crystalline form. In another aspect, the crystalline form of the compound comprises at least about 99 weight % of the desired crystalline form.

In some embodiments, the present disclosure provides a crystalline form of (R)—N-(2-(4-cyanothiazolidin-3-yl)-2-oxoethyl)-6-morpholinoquinoline-4-carboxamide which has the following chemical structure:

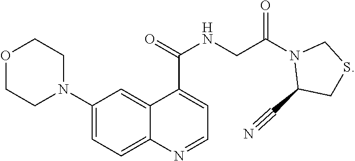 [SMILES: N#C[C@@H]1CSCN1C(=O)CNC(=O)c1ccnc2ccc(N3CCOCC3)cc12]

In some embodiments, the present disclosure provides crystalline (R)—N-(2-(4-cyanothiazolidin-3-yl)-2-oxoethyl)-6-morpholinoquinoline-4-carboxamide Form A.

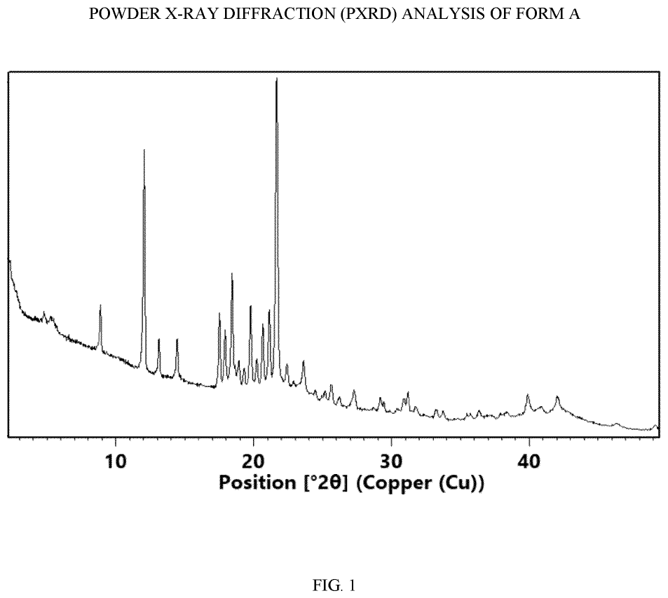

In some embodiments, Form A is characterized by a transmission X-ray powder diffraction pattern: (i) that comprises at least one peak selected from the group consisting of 12.0±0.2° 2θ, 18.4±0.2° 2θ, 19.8±0.2° 2θ, and 21.7°2θ±0.2° 2θ, and (ii) that does not comprise peaks at 18.7±0.2° 2θ and 22.4° 2θ±0.2° 2θ having a medium or stronger relative intensity. In one aspect, the transmission X-ray powder diffraction pattern does not comprise peaks at 18.7±0.2° 2θ and 22.4° 2θ±0.2° 2θ. In another aspect, the transmission X-ray powder diffraction pattern comprises at least two, three, or four peaks selected from the group consisting of 12.0±0.2° 2θ, 18.4±0.2° 2θ, 19.8±0.2° 2θ, and 21.7° 2θ±0.2° 2θ. In another aspect, the transmission X-ray powder diffraction pattern comprises peaks at 12.0±0.2° 2θ, 18.4±0.2° 2θ, 19.8±0.2° 2θ, and 21.7° 2θ±0.2° 2θ. In another aspect, the transmission X-ray powder diffraction pattern further comprises at least one, two, three, or four peaks selected from the group consisting of 13.1±0.2° 2θ, 14.4±0.2° 2θ, 17.5±0.2° 2θ, and 21.1° 2θ±0.2° 2θ. In another aspect, the transmission X-ray powder diffraction pattern further comprises peaks at 13.1±0.2° 2θ, 14.4±0.2° 2θ, 17.5±0.2° 2θ, and 21.1°2θ±0.2° 2θ. In another aspect, the transmission X-ray powder diffraction pattern comprises peaks at 8.9±0.2° 2θ, 12.0±0.2° 2θ, 13.1±0.2° 2θ, 14.4±0.2° 2θ, 17.5±0.2° 2θ, 17.9±0.2° 2θ, 18.4±0.2° 2θ, 19.8±0.2° 2θ, 20.6±0.2° 2θ, 21.1±0.2° 2θ, 21.7±0.2° 2θ. 23.6±0.2° 2θ, 25.6±0.2° 2θ, 27.3±0.2° 2θ, 31.2±0.2° 2θ, 39.9±0.2° 2θ, and 42.0±0.2° 2θ. In another aspect, the transmission X-ray powder diffraction pattern is substantially the same as the transmission X-ray powder diffraction pattern of FIG. 1.

In some embodiments, Form A is characterized by a solid-state ¹³C NMR spectrum comprising at least one peak selected from the group consisting of 166.1±0.2 ppm, 130.7±0.2 ppm, 117.9±0.2 ppm, 108.6±0.2 ppm, and 35.2±0.2 ppm. In one aspect, the solid-state ¹³C NMR spectrum comprises peaks at 166.1±0.2 ppm, 117.9±0.2 ppm, and 108.6±0.2 ppm. In another aspect, the solid-state ¹³C NMR spectrum comprises peaks at 166.1±0.2 ppm, 130.7±0.2 ppm, 117.9±0.2 ppm, 108.6±0.2 ppm, and 35.2±0.2 ppm. In another aspect, the solid-state ¹³C NMR spectrum comprises peaks at 168.0±0.2 ppm, 166.1±0.2 ppm, 147.5±0.2 ppm, 146.4±0.2 ppm, 143.1±0.2 ppm, 139.5±0.2 ppm, 130.7±0.2 ppm, 125±0.2 ppm, 117.9±0.2 ppm, 108.6±0.2 ppm, 67.1±0.2 ppm, 48.0±0.2 ppm, 42.9±0.2 ppm, and 35.2±0.2 ppm. In another aspect, the solid-state ¹³C NMR spectrum does not comprise at least one peak selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm. In another aspect, the solid-state ¹³C NMR spectrum does not comprise at least five peaks selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm. In another aspect, the solid-state ¹³C NMR spectrum does not comprise peaks at 49.9±0.2 ppm and 46.6±0.2 ppm. In another aspect, the solid-state ¹³C NMR spectrum does not comprise peaks at 166.9±0.2 ppm, 118.6±0.2 ppm, 110.0±0.2 ppm, 49.9±0.2 ppm, and 46.6±0.2 ppm. In another aspect, the solid-state ¹³C NMR spectrum does not comprise peaks at 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

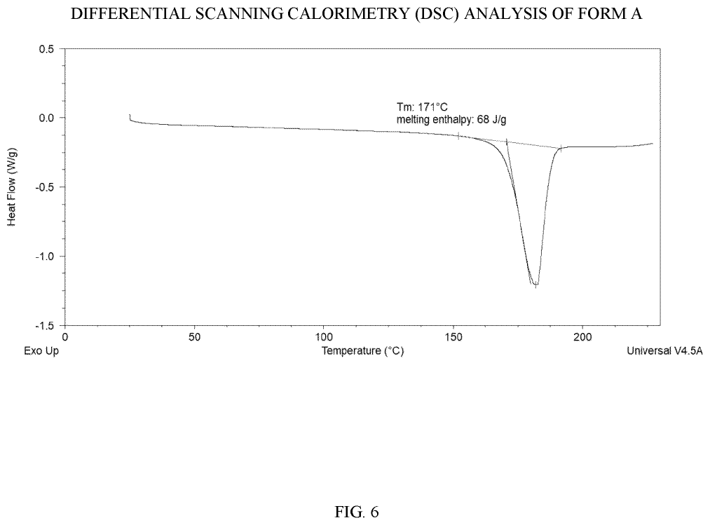

In some embodiments, Form A is characterized by a differential scanning calorimetry curve comprising a melting endotherm having an onset temperature between about 165° C. to about 180° C. In one aspect, the endotherm comprises a melting endotherm having an onset temperature between about 165° C. to about 177° C. In another aspect, the endotherm has an onset at 171° C.±5° C. In another aspect, the differential scanning calorimetry curve is substantially the same as the differential scanning calorimetry curve of FIG. 6.

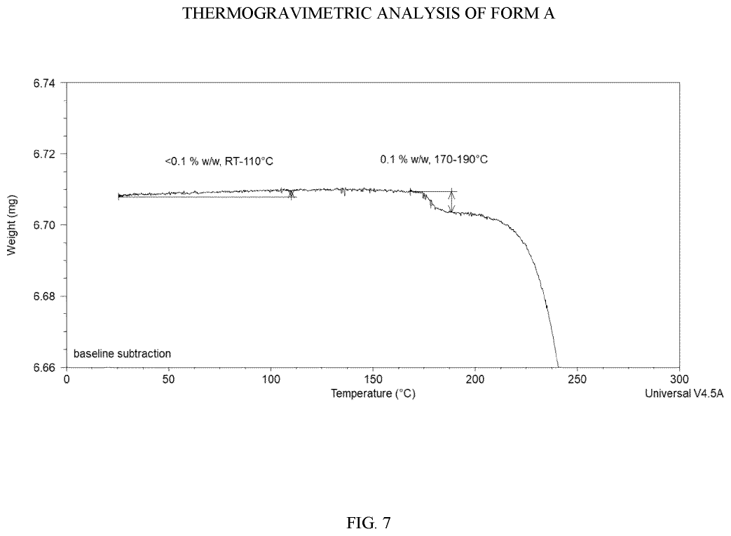

In some embodiments, Form A is characterized by a thermogravimetric analysis thermogram wherein the crystalline form exhibits a weight loss of less than about 0.5 weight % from about 25° C. to about 110° C. In one aspect, the weight loss is less than about 0.2 weight %. In another aspect, the weight loss is less than about 0.1 weight %. In another aspect, the thermogravimetric analysis thermogram is substantially the same as the thermogravimetric analysis thermogram of FIG. 7.

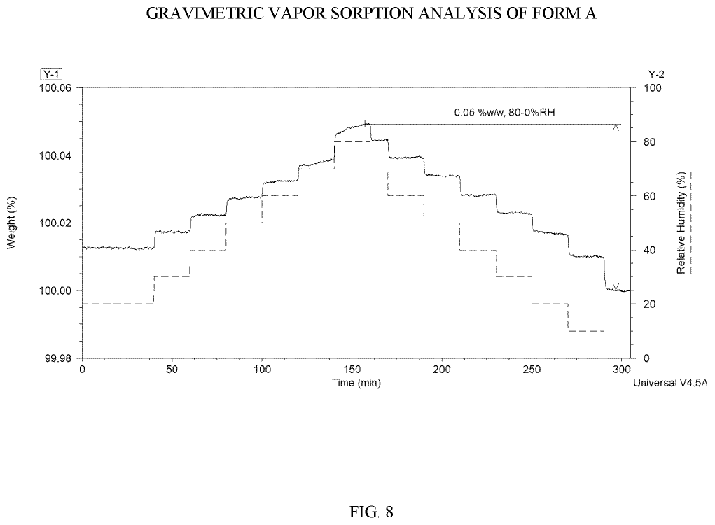

In some embodiments, Form A is characterized by a gravimetric vapor sorption plot wherein the crystalline form exhibits a reversible moisture uptake of less than about 0.5 weight % from about 0% relative humidity to about 80% relative humidity at 25° C.±0.1° C. In one aspect, the reversible moisture uptake is less than about 0.2 weight %. In another aspect, the reversible moisture uptake is less than about 0.1 weight %. In another aspect, the gravimetric vapor sorption plot is substantially the same as the gravimetric vapor sorption plot of FIG. 8.

In some embodiments, Form A is characterized by at least two of the above-described physical characterization embodiments (transmission X-ray powder diffraction, solid-state ¹³C NMR, differential scanning calorimetry, thermogravimetric analysis, and/or gravimetric vapor sorption).

In some embodiments, Form A is characterized by at least three of the above-described physical characterization embodiments (transmission X-ray powder diffraction, solid-state ¹³C NMR, differential scanning calorimetry, thermogravimetric analysis, and/or gravimetric vapor sorption).

In some embodiments, Form A is characterized by at least four of the above-described physical characterization embodiments (transmission X-ray powder diffraction, solid-state ¹³C NMR, differential scanning calorimetry, thermogravimetric analysis, and/or gravimetric vapor sorption).

In some embodiments, Form A is characterized by the following:
a transmission X-ray powder diffraction pattern that comprises at least one peak selected from the group consisting of 12.0±0.2° 2θ, 18.4±0.2° 2θ, 19.8±0.2° 2θ, and 21.7° 2θ±0.2° 2θ, and that does not comprise peaks at 18.7±0.2° 2θ and 22.4° 2θ+0.2° 2θ having a medium or stronger relative intensity;
a solid-state ¹³C NMR spectrum comprising peaks at 166.1±0.2 ppm, 130.7±0.2 ppm, 117.9±0.2 ppm, 108.6±0.2 ppm, and 35.2±0.2 ppm; and
a differential scanning calorimetry curve comprising a melting endotherm having an onset temperature between about 165° C. to about 180° C.

In some embodiments, Form A is characterized by the following:
a transmission X-ray powder diffraction pattern that comprises at least one peak selected from the group consisting of 12.0±0.2° 2θ, 18.4±0.2° 2θ, 19.8±0.2° 2θ, and 21.7° 2θ±0.2° 2θ, and that does not comprise peaks at 18.7±0.2° 2θ and 22.4° 2θ+0.2° 2θ having a medium or stronger relative intensity:
a solid-state ¹³C NMR spectrum comprising peaks at 166.1±0.2 ppm, 130.7±0.2 ppm, 117.9±0.2 ppm, 108.6±0.2 ppm, and 35.2±0.2 ppm;
a differential scanning calorimetry curve comprising a melting endotherm having an onset temperature between about 165° C. to about 177° C.; and
a thermogravimetric analysis thermogram wherein the crystalline form exhibits a weight loss of less than about 0.2 weight % from about 25° C. to about 110° C.

In some embodiments, Form A is characterized by the following:
a transmission X-ray powder diffraction pattern that comprises at least one peak selected from the group consisting of 12.0±0.2° 2θ, 18.4±0.2° 2θ, 19.8±0.2° 2θ, and 21.7°2θ+0.2° 2θ, and that does not comprise peaks at 18.7±0.2° 2θ and 22.4° 2θ±0.2° 2θ having a medium or stronger relative intensity:
a solid-state ¹³C NMR spectrum comprising peaks at 166.1±0.2 ppm, 130.7±0.2 ppm, 117.9±0.2 ppm, 108.6±0.2 ppm, and 35.2±0.2 ppm;
a differential scanning calorimetry curve comprising a melting endotherm having an onset temperature between about 165° C. to about 177° C.; and
a gravimetric vapor sorption plot wherein the crystalline form exhibits a reversible moisture uptake of less than about 0.2 weight % from about 0% relative humidity to about 80% relative humidity at 25° C.±0.1° C.

In some embodiments, Form A is characterized by the following:
a transmission X-ray powder diffraction pattern that comprises at least one peak selected from the group consisting of 12.0±0.2° 2θ, 18.4±0.2° 2θ, 19.8±0.2° 2θ, and 21.7°2θ±0.2° 2θ, and that does not comprise peaks at 18.7±0.2° 2θ and 22.4° 2θ±0.2° 2θ having a medium or stronger relative intensity:
a solid-state ¹³C NMR spectrum comprising peaks at 166.1±0.2 ppm, 130.7±0.2 ppm, 117.9±0.2 ppm, 108.6±0.2 ppm, and 35.2±0.2 ppm:
a differential scanning calorimetry curve comprising a melting endotherm having an onset at 171° C.±5° C.;
a thermogravimetric analysis thermogram wherein the crystalline form exhibits a weight loss of less than about 0.2 weight % from about 25° C. to about 110° C.; and
a gravimetric vapor sorption plot wherein the crystalline form exhibits a reversible moisture uptake of less than about 0.2 weight % from about 0% relative humidity to about 80% relative humidity at 25° C.±0.1° C.

In some embodiments, Form A is a crystalline anhydrate.

In some embodiments, Form A has a long needle morphology.

In some embodiments, Form A is substantially free of any other crystalline form of (R)—N-(2-(4-cyanothiazolidin-3-yl)-2-oxoethyl)-6-morpholinoquinoline-4-carboxamide.

In some embodiments, Form A comprises less than 5 weight % of any other crystalline form of (R)—N-(2-(4-cyanothiazolidin-3-yl)-2-oxoethyl)-6-morpholinoquinoline-4-carboxamide.

In some embodiments, Form A comprises less than 10 weight % of any other crystalline form of (R)—N-(2-(4-cyanothiazolidin-3-yl)-2-oxoethyl)-6-morpholinoquinoline-4-carboxamide.

In some embodiments, the present disclosure provides crystalline (R)—N-(2-(4-cyanothiazolidin-3-yl)-2-oxoethyl)-6-morpholinoquinoline-4-carboxamide Form B.

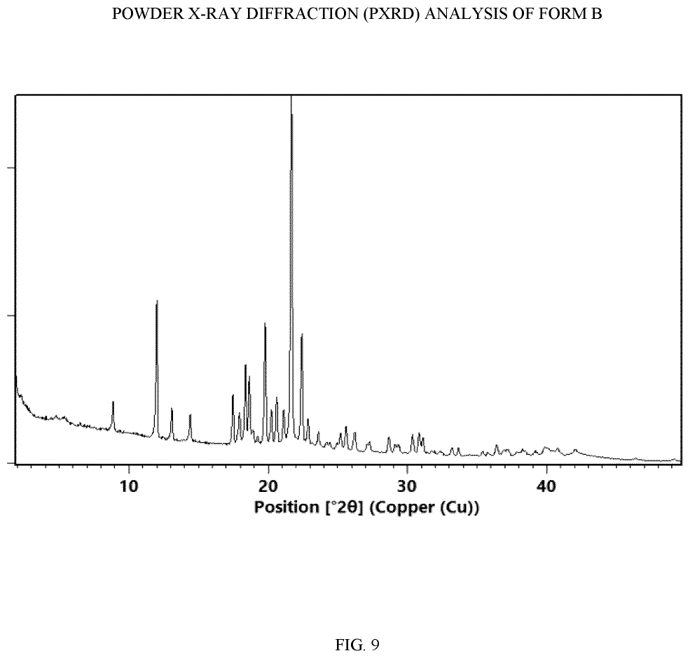

In some embodiments, Form B is characterized by a transmission X-ray powder diffraction pattern comprising peaks at 18.7±0.2° 2θ and 22.4° 2θ±0.2° 2θ. In one aspect, the peaks at 18.7±0.2° 2θ and 22.4° 2θ+0.2° 2θ have at least a medium or stronger relative intensity. In another aspect, the transmission X-ray powder diffraction pattern further comprises at least one, two, three, or four peaks selected from the group consisting of 12.0±0.2° 2θ, 18.4±0.2° 2θ, 19.8±0.2° 2θ, and 21.7° 2θ±0.2° 2θ. In another aspect, the transmission X-ray powder diffraction pattern further comprises peaks at 12.0±0.2° 2θ, 18.4±0.2° 2θ, 19.8±0.2° 2θ, and 21.7° 2θ±0.2° 2θ. In another aspect, the transmission X-ray powder diffraction pattern further comprises at least one, two, three, or four peaks selected from the group consisting of 13.1±0.2° 2θ, 14.4±0.2° 2θ, 17.5±0.2° 2θ, and 21.1° 2θ+0.2° 2θ. In another aspect, the transmission X-ray powder diffraction pattern further comprises peaks at 13.1±0.2° 2θ, 14.4±0.2° 2θ, 17.5±0.2° 2θ, and 21.1° 2θ±0.2° 2θ. In another aspect, the transmission X-ray powder diffraction pattern comprises peaks at 8.9° 2θ±0.2° 2θ, 12.0° 2θ±0.2° 2θ, 13.1° 2θ±0.2° 2θ, 14.4° 2θ±0.2° 2θ, 17.5° 2θ±0.2° 2θ, 18.0° 2θ±0.2° 2θ, 18.4° 2θ±0.2° 2θ, 18.7° 2θ±0.2° 2θ, 19.8° 2θ±0.2° 2θ, 20.3°2θ±0.2° 2θ, 20.6° 2θ±0.2° 2θ, 21.1° 2θ±0.2° 2θ, 21.7° 2θ±0.2° 2θ, 22.4° 2θ±0.2° 2θ, 22.9° 2θ±0.2° 2θ, 25.2° 2θ±0.2° 2θ, 25.6° 2θ±0.2° 2θ, 26.2° 2θ±0.2° 2θ, 28.7°2θ±0.2° 2θ, 30.4° 2θ±0.2° 2θ, 30.9° 2θ±0.2° 2θ, and 31.1° 2θ±0.2° 2θ. In another aspect, the transmission X-ray powder diffraction pattern is substantially the same as the transmission X-ray powder diffraction pattern of FIG. 9.

In some embodiments, Form B is characterized by a solid-state ¹³C NMR spectrum comprising at least one peak selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm. In one aspect, the solid-state ¹³C NMR spectrum comprises peaks at 49.9±0.2 ppm and 46.6±0.2 ppm. In another aspect, the solid-state ¹³C NMR spectrum comprises at least five peaks selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm. In another aspect, the solid-state ¹³C NMR spectrum comprises peaks at 166.9±0.2 ppm, 118.6±0.2 ppm, 110.0±0.2 ppm, 49.9±0.2 ppm, and 46.6±0.2 ppm. In another aspect, the solid-state ¹³C NMR spectrum comprises peaks at 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm. In another aspect, the solid-state ¹³C NMR spectrum comprises peaks at 167.7±0.2 ppm, 166.9±0.2 ppm, 147.3±0.2 ppm, 143.1±0.2 ppm, 139.9±0.2 ppm, 130.2±0.2 ppm, 125±0.2 ppm, 119.5±0.2 ppm, 118.6±0.2 ppm, 117.4±0.2 ppm, 110.0±0.2 ppm, 49.9±0.2 ppm, 46.6±0.2 ppm, and 34.5±0.2 ppm. In another aspect, the solid-state ¹³C NMR spectrum does not comprise at least one peak selected from the group consisting of 166.1±0.2 ppm, 130.7±0.2 ppm, 117.9±0.2 ppm, 108.6±0.2 ppm, and 35.2±0.2 ppm. In another aspect, the solid-state ¹³C NMR spectrum does not comprise peaks at 166.1±0.2 ppm, 117.9±0.2 ppm, and 108.6±0.2 ppm. In another aspect, the solid-state ¹³C NMR spectrum does not comprise peaks at 166.1±0.2 ppm, 130.7±0.2 ppm, 117.9±0.2 ppm, 108.6±0.2 ppm, and 35.2±0.2 ppm.

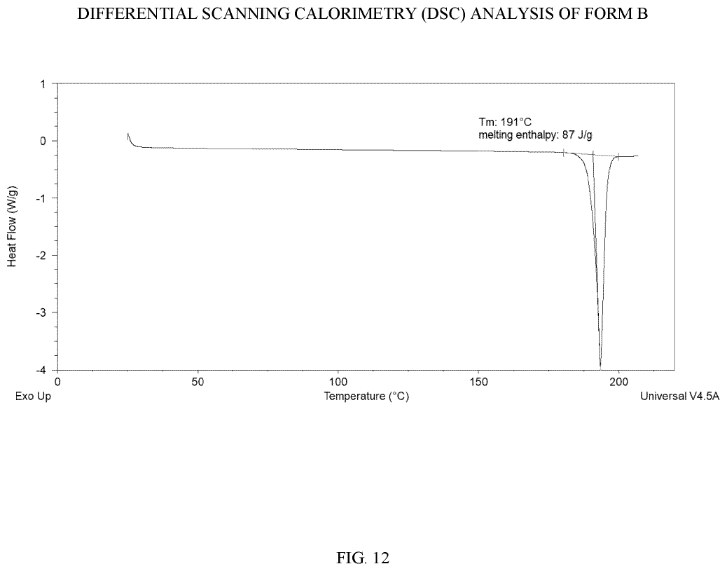

In some embodiments, Form B is characterized by a differential scanning calorimetry curve comprising a melting endotherm having an onset between about 185° C. to about 200° C. In one aspect, the endotherm has an onset between about 185° C. to about 197° C. In another aspect, the endotherm has an onset at 191° C.±5° C. In another aspect, the differential scanning calorimetry curve is substantially the same as the differential scanning calorimetry curve of FIG. 12.

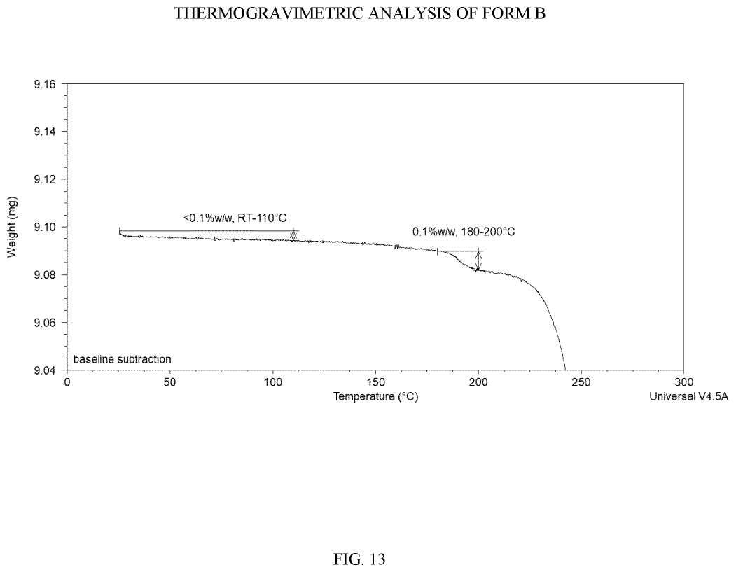

In some embodiments, Form B is characterized by a thermogravimetric analysis thermogram wherein the crystalline form exhibits a weight loss of less than about 0.5 weight % from about 25° C. to about 110° C. In one aspect, the weight loss is less than about 0.2 weight %. In another aspect, the weight loss is less than about 0.1 weight %. In another aspect, the thermogravimetric analysis thermogram is substantially the same as the thermogravimetric analysis thermogram of FIG. 13.

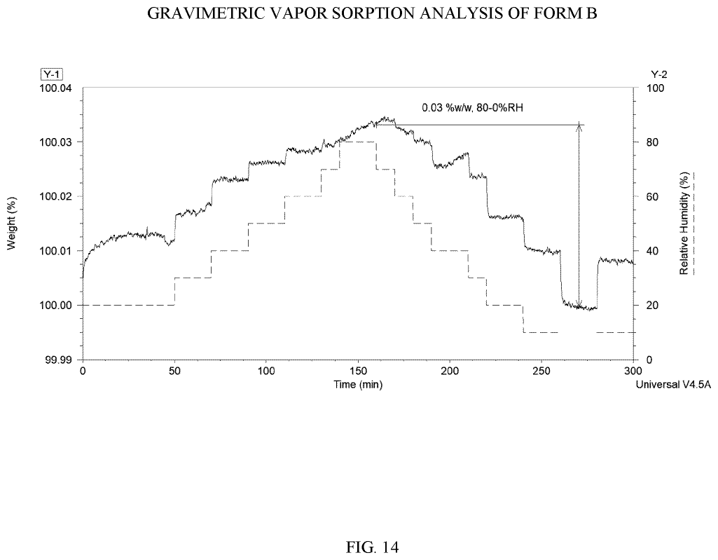

In some embodiments, Form B is characterized by a gravimetric vapor sorption plot wherein the crystalline form exhibits a reversible moisture uptake of less than about 0.5 weight % from about 0% relative humidity to about 80% relative humidity at 25° C.±0.1° C. In one aspect, the reversible moisture uptake is less than about 0.2 weight %. In another aspect, the reversible moisture uptake is less than about 0.1 weight %. In another aspect, the gravimetric vapor sorption plot is substantially the same as the gravimetric vapor sorption plot of FIG. 14.

In some embodiments, Form B is characterized by at least two of the above-described physical characterization embodiments (transmission X-ray powder diffraction, solid-state ¹³C NMR, differential scanning calorimetry, thermogravimetric analysis, and/or gravimetric vapor sorption).

In some embodiments, Form B is characterized by at least three of the above-described physical characterization embodiments (transmission X-ray powder diffraction, solid-state ¹³C NMR, differential scanning calorimetry, thermogravimetric analysis, and/or gravimetric vapor sorption).

In some embodiments, Form B is characterized by at least four of the above-described physical characterization embodiments (transmission X-ray powder diffraction, solid-state ¹³C NMR, differential scanning calorimetry, thermogravimetric analysis, and/or gravimetric vapor sorption).

In some embodiments, Form B is characterized by the following:
a transmission X-ray powder diffraction pattern comprising peaks at 18.7±0.2° 2θ and 22.4° 2θ±0.2° 2θ;
a solid-state ¹³C NMR spectrum comprising at least five peaks selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm; and a differential scanning calorimetry curve comprising a melting endotherm having an onset between about 185° C. to about 200° C.

In some embodiments, Form B is characterized by the following:
a transmission X-ray powder diffraction pattern comprising peaks at 18.7±0.2° 2θ and 22.4° 2θ±0.2° 2θ;
a solid-state ¹³C NMR spectrum comprising peaks at 166.9±0.2 ppm, 118.6±0.2 ppm, 110.0±0.2 ppm, 49.9±0.2 ppm, and 46.6±0.2 ppm;
a differential scanning calorimetry curve comprising a melting endotherm having an onset between about 185° C. to about 200° C.; and
a thermogravimetric analysis thermogram wherein the crystalline form exhibits a weight loss of less than about 0.2 weight % from about 25° C. to about 110° C.

In some embodiments, Form B is characterized by the following:
a transmission X-ray powder diffraction pattern comprising peaks at 18.7±0.2° 2θ and 22.4° 2θ±0.2° 2θ;
a solid-state ¹³C NMR spectrum comprising peaks at 166.9±0.2 ppm, 118.6±0.2 ppm, 110.0±0.2 ppm, 49.9±0.2 ppm, and 46.6±0.2 ppm:
a differential scanning calorimetry curve comprising a melting endotherm having an onset between about 185° C. to about 200° C.; and
a gravimetric vapor sorption plot wherein the crystalline form exhibits a reversible moisture uptake of less than about 0.2 weight % from about 0% relative humidity to about 80% relative humidity at 25° C.±0.1° C.

In some embodiments, Form B is characterized by the following:
a transmission X-ray powder diffraction pattern comprising peaks at 18.7±0.2° 2θ and 22.4° 2θ±0.2° 2θ;
a solid-state ¹³C NMR spectrum comprising peaks at 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm:
a differential scanning calorimetry curve comprising a melting endotherm having an onset at 191° C.±5° C.;
a thermogravimetric analysis thermogram wherein the crystalline form exhibits a weight loss of less than about 0.1 weight % from about 25° C. to about 110° C.; and
a gravimetric vapor sorption plot wherein the crystalline form exhibits a reversible moisture uptake of less than about 0.1 weight % from about 0% relative humidity to about 80% relative humidity at 25° C.±0.1° C.

In some embodiments, Form B is a crystalline anhydrate.

In some embodiments, Form B has a long needle morphology.

In some embodiments, Form B is substantially free of any other crystalline form of (R)—N-(2-(4-cyanothiazolidin-3-yl)-2-oxoethyl)-6-morpholinoquinoline-4-carboxamide.

In some embodiments, Form B comprises less than 5 weight % of any other crystalline form of (R)—N-(2-(4-cyanothiazolidin-3-yl)-2-oxoethyl)-6-morpholinoquinoline-4-carboxamide.

In some embodiments, Form B comprises less than 10 weight % of any other crystalline form of (R)—N-(2-(4-cyanothiazolidin-3-yl)-2-oxoethyl)-6-morpholinoquinoline-4-carboxamide.

In some embodiments the present disclosure provides a crystalline form of (R,S)—N-(2-(4-cyanothiazolidin-3-yl)-2-oxoethyl)-6-morpholinoquinoline-4-carboxamide (i.e., a crystalline form of racemic N-(2-(4-cyanothiazolidin-3-yl)-2-oxoethyl)-6-morpholino-quinoline-4-carboxamide).

In some embodiments, the present disclosure provides crystalline (R,S)—N-(2-(4-cyanothiazolidin-3-yl)-2-oxoethyl)-6-morpholinoquinoline-4-carboxamide Type 2.

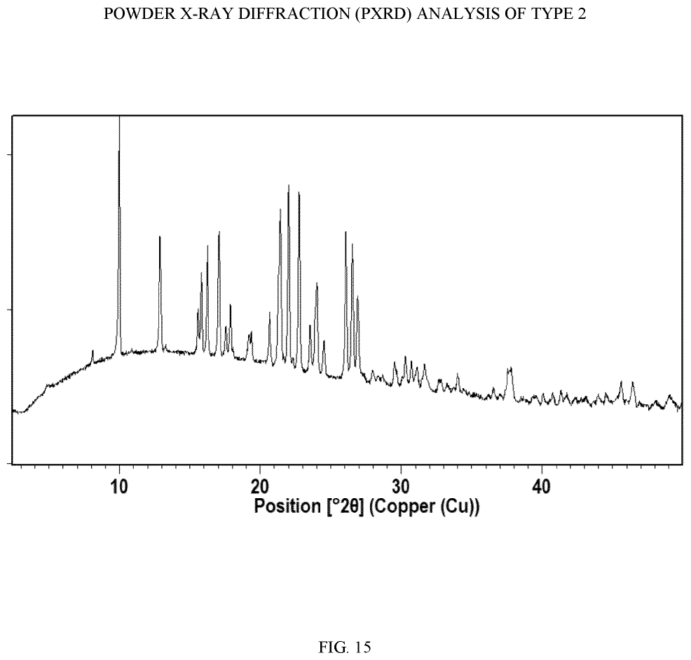

In some embodiments, Type 2 is characterized by a reflection X-ray powder diffraction pattern comprising at least one peak selected from the group consisting of 10.0±0.2° 2θ, 12.9±0.2° 2θ, 17.1±0.2° 2θ, 22.0±0.2° 2θ, and 22.8° 2θ±0.2° 2θ. In one aspect, the reflection X-ray powder diffraction pattern comprises peaks at 10.0±0.2° 2θ, 12.9±0.2° 2θ, 17.1±0.2° 2θ, 22.0±0.2° 2θ, and 22.8° 2θ±0.2° 2θ. In another aspect, the reflection X-ray powder diffraction pattern further comprises at least one, two, three, four, or five peaks selected from the group consisting of 15.8±0.2° 2θ, 16.2±0.2° 2θ, 26.0±0.2° 2θ, 26.5±0.2° 2θ, and 26.9° 2θ±0.2° 2θ. In another aspect, the reflection X-ray powder diffraction pattern further comprises peaks at 15.8±0.2° 2θ, 16.2±0.2° 2θ, 26.0±0.2° 2θ, 26.5±0.2° 2θ, and 26.9° 2θ±0.2° 2θ. In another aspect, the reflection X-ray powder diffraction pattern comprises peaks at 8.1±0.2° 2θ, 10.0±0.2° 2θ, 12.9±0.2° 2θ, 15.6±0.2° 2θ, 15.8±0.2° 2θ, 16.2±0.2° 2θ, 17.1±0.2° 2θ, 17.6±0.2° 2θ, 17.9±0.2° 2θ, 19.2±0.2° 2θ, 19.3±0.2° 2θ, 20.7±0.2° 2θ, 21.4±0.2° 2θ, 22.0±0.2° 2θ, 22.8±0.2° 2θ, 23.5±0.2° 2θ, 24.0±0.2° 2θ, 24.5±0.2° 2θ, 26.1±0.2° 2θ, 26.5±0.2° 2θ, 26.9±0.2° 2θ, 29.5±0.2° 2θ, 30.3±0.2° 2θ, 30.7±0.2° 2θ, 31.1±0.2° 2θ, 31.7±0.2° 2θ, 32.7±0.2° 2θ, 34.0±0.2° 2θ, and 37.7±0.2° 2θ. In another aspect, the reflection X-ray powder diffraction pattern is substantially the same as the reflection X-ray powder diffraction pattern of FIG. 15.

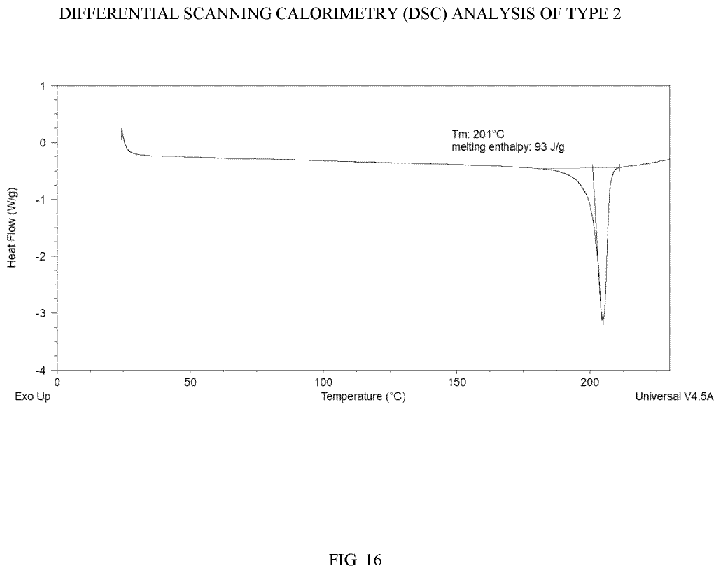

In some embodiments, Type 2 is characterized by a differential scanning calorimetry curve comprising a melting endotherm between about 195° C. to about 210° C. In one aspect, the endotherm has an onset temperature between about 195° C. to about 207° C. In another aspect, the endotherm has an onset temperature at 201° C.±5° C. In another aspect, the differential scanning calorimetry curve is substantially the same as the differential scanning calorimetry curve of FIG. 16.

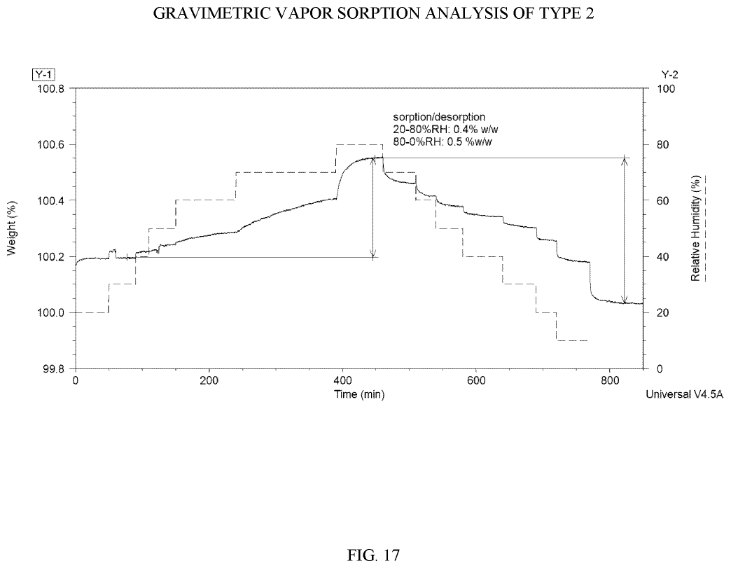

In some embodiments, Type 2 is characterized by a gravimetric vapor sorption plot wherein the crystalline form exhibits a reversible moisture uptake of less than about 1.0 weight % from about 0% relative humidity to about 80% relative humidity at 25° C.±0.1° C. In one aspect, the reversible moisture uptake is less than about 0.7 weight %. In another aspect, the reversible moisture uptake is about 0.5 weight %. In another aspect, the gravimetric vapor sorption plot is substantially the same as the gravimetric vapor sorption plot of FIG. 17.

In some embodiments, Type 2 is characterized by at least two of the above-described physical characterization embodiments (reflection X-ray powder diffraction, differential scanning calorimetry, and/or gravimetric vapor sorption).

In some embodiments, Type 2 is characterized by at least three of the above-described physical characterization embodiments (reflection X-ray powder diffraction, differential scanning calorimetry, and gravimetric vapor sorption).

In some embodiments, Type 2 is characterized by the following:
a reflection X-ray powder diffraction pattern comprises peaks at 10.0±0.2° 2θ, 12.9±0.2° 2θ, 17.1±0.2° 2θ, 22.0±0.2° 2θ, and 22.8° 2θ±0.2° 2θ;
a differential scanning calorimetry curve comprising a melting endotherm having an onset temperature between about 190° C. to about 210° C.; and
a gravimetric vapor sorption plot wherein the crystalline form exhibits a reversible moisture uptake of less than about 1.0 weight % from about 0% relative humidity to about 80% relative humidity at 25° C.±0.1° C.

In some embodiments, Type 2 is characterized by the following:
a reflection X-ray powder diffraction pattern comprises peaks at 10.0±0.2° 2θ, 12.9±0.2° 2θ, 17.1±0.2° 2θ, 22.0±0.2° 2θ, and 22.8° 2θ±0.2° 2θ;
a differential scanning calorimetry curve comprising a melting endotherm having an onset temperature between about 195° C. to about 207° C.; and
a gravimetric vapor sorption plot wherein the crystalline form exhibits a reversible moisture uptake of less than about 0.7 weight % from about 0% relative humidity to about 80% relative humidity at 25° C.±0.1° C.

In some embodiments, Type 2 is characterized by the following:
a reflection X-ray powder diffraction pattern comprises peaks at 10.0±0.2° 2θ, 12.9±0.2° 2θ, 17.1±0.2° 2θ, 22.0±0.2° 2θ, and 22.8° 2θ±0.2° 2θ;
a differential scanning calorimetry curve comprising a melting endotherm having an onset at 201° C.±5° C.; and
a gravimetric vapor sorption plot wherein the crystalline form exhibits a reversible moisture uptake of about 0.5 weight % from about 0% relative humidity to about 80% relative humidity at 25° C.±0.1° C.

In some embodiments, Type 2 is a crystalline anhydrate.

In some embodiments, Type 2 is substantially free of any other crystalline form of N-(2-(4-cyanothiazolidin-3-yl)-2-oxoethyl)-6-morpholinoquinoline-4-carboxamide.

In some embodiments, Type 2 comprises less than 5 weight % of any other crystalline form of N-(2-(4-cyanothiazolidin-3-yl)-2-oxoethyl)-6-morpholinoquinoline-4-carboxamide.

In some embodiments, Type 2 comprises less than 10 weight % of any other crystalline form of N-(2-(4-cyanothiazolidin-3-yl)-2-oxoethyl)-6-morpholinoquinoline-4-carboxamide.

In some embodiments the present disclosure provides a composition comprising at least two crystalline forms selected from the group consisting of Form A, Form B, and Type 2.

In some embodiments, the present disclosure provides a composition comprising Form A and Form B. In one aspect, the composition comprises at least 50 weight % of Form B relative to Form A. In another aspect, the composition comprises at least 75 weight % of Form B relative to Form A. In another aspect, the composition comprises at least 90 weight % of Form B relative to Form A. In another aspect, the composition comprises at least 95 weight % of Form B relative to Form A. In another aspect, the composition comprises at least 95 weight % of Form B relative to any other crystalline forms.

### Representative Embodiments

Embodiment 1: A crystalline form of N-(2-(4-cyanothiazolidin-3-yl)-2-oxoethyl)-6-morpholinoquinoline-4-carboxamide.

Embodiment 2: The crystalline form of Embodiment 1 that is a crystalline form of (R)—N-(2-(4-cyanothiazolidin-3-yl)-2-oxoethyl)-6-morpholinoquinoline-4-carboxamide.

Embodiment 3: The crystalline form of Embodiment 2 characterized by a transmission X-ray powder diffraction pattern comprising peaks at 18.7±0.2° 2θ and 22.4° 2θ±0.2° 2θ.

Embodiment 4: The crystalline form of Embodiment 3, wherein the peaks at 18.7±0.2° 2θ and 22.4° 2θ±0.2° 2θ have at least a medium or stronger relative intensity.

Embodiment 5: The crystalline form of Embodiment 3, wherein the transmission X-ray powder diffraction pattern further comprises at least one peak selected from the group consisting of 12.0±0.2° 2θ, 18.4±0.2° 2θ, 19.8±0.2° 2θ, and 21.7° 2θ±0.2° 2θ.

Embodiment 6: The crystalline form of Embodiment 3, wherein the transmission X-ray powder diffraction pattern further comprises peaks at 12.0±0.2° 2θ, 18.4±0.2° 2θ, 19.8±0.2° 2θ, and 21.7° 2θ±0.2° 2θ.

Embodiment 7: The crystalline form of Embodiment 6, wherein the transmission X-ray powder diffraction pattern further comprises at least one peak selected from the group consisting of 13.1±0.2° 2θ, 14.4±0.2° 2θ, 17.5±0.2° 2θ, and 21.1° 2θ±0.2° 2θ.

Embodiment 8: The crystalline form of Embodiment 6, wherein the transmission X-ray powder diffraction pattern further comprises peaks at 13.1±0.2° 2θ, 14.4±0.2° 2θ, 17.5±0.2° 2θ, and 21.1° 2θ±0.2° 2θ.

Embodiment 9: The crystalline form of any of Embodiments 3 to 8, wherein the transmission x-ray powder diffraction is carried out using Cu radiation.

Embodiment 10: The crystalline form of any of Embodiments 3 to 9, wherein the transmission x-ray powder diffraction is carried out using a PANalytical Empyrean diffractometer operating in transmission geometry, a tube voltage of 45 kV, and filament emission of 40 mA.

Embodiment 11: The crystalline form of any of Embodiments 2 to 10 further characterized by a solid-state ¹³C NMR spectrum comprising at least one peak selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

Embodiment 12: The crystalline form of Embodiment 11, wherein the solid-state ¹³C NMR spectrum comprises peaks at 49.9±0.2 ppm and 46.6±0.2 ppm.

Embodiment 13: The crystalline form of Embodiment 11, wherein the solid-state ¹³C NMR spectrum comprises at least five peaks selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

Embodiment 14: The crystalline form of Embodiment 11, wherein the solid-state ¹³C NMR spectrum comprises peaks at 166.9±0.2 ppm, 118.6±0.2 ppm, 110.0±0.2 ppm, 49.9±0.2 ppm, and 46.6±0.2 ppm.

Embodiment 15: The crystalline form of Embodiment 11, wherein the solid-state ¹³C NMR spectrum comprises peaks at 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

Embodiment 16: The crystalline form of Embodiment 11, wherein the solid-state ¹³C NMR spectrum comprises peaks at 167.7±0.2 ppm, 166.9±0.2 ppm, 147.3±0.2 ppm, 143.1±0.2 ppm, 139.9±0.2 ppm, 130.2±0.2 ppm, 125±0.2 ppm, 119.5±0.2 ppm, 118.6±0.2 ppm, 117.4±0.2 ppm, 110.0±0.2 ppm, 49.9±0.2 ppm, 46.6±0.2 ppm, and 34.5±0.2 ppm.

Embodiment 17: The crystalline form of any of Embodiments 11 to 16, wherein the solid-state ¹³C NMR spectrum does not comprise at least one peak selected from the group consisting of 166.1±0.2 ppm, 130.7±0.2 ppm, 117.9±0.2 ppm, 108.6±0.2 ppm, and 35.2±0.2 ppm.

Embodiment 18: The crystalline form of any of Embodiments 11 to 16, wherein the solid-state ¹³C NMR spectrum does not comprise peaks at 166.1±0.2 ppm, 117.9±0.2 ppm, and 108.6±0.2 ppm.

Embodiment 19: The crystalline form of any of Embodiments 11 to 16, wherein the solid-state ¹³C NMR spectrum does not comprise peaks at 166.1±0.2 ppm, 130.7±0.2 ppm, 117.9±0.2 ppm, 108.6±0.2 ppm, and 35.2±0.2 ppm.

Embodiment 20: The crystalline form of any of Embodiments 2 to 19 further characterized by a differential scanning calorimetry curve comprising a melting endotherm having an onset between about 185° C. to about 200° C.

Embodiment 21: The crystalline form of Embodiment 20, wherein the endotherm has an onset between about 185° C. to about 197° C.

Embodiment 22: The crystalline form of Embodiment 20, wherein the endotherm has an onset at 191° C.±5° C.

Embodiment 23: The crystalline form of Embodiment 20, wherein the differential scanning calorimetry curve is substantially the same as the differential scanning calorimetry curve of FIG. 12.

Embodiment 24: The crystalline form of any of Embodiments 20 to 23, wherein the differential scanning calorimetry is conducted on a TA Instruments Differential Scanning calorimeter, model Q2000, with a sample placed in an aluminum pan and heated under nitrogen at a rate of 10° C./minute to a temperature of 230° C.

Embodiment 25: The crystalline form of any of Embodiments 3 to 24 further characterized by a thermogravimetric analysis thermogram wherein the crystalline form exhibits a weight loss of less than about 0.5 weight % from about 25° C. to about 110° C.

Embodiment 26: The crystalline form of Embodiment 25, wherein the weight loss is less than about 0.2 weight %.

Embodiment 27: The crystalline form of Embodiment 25, wherein the weight loss is less than about 0.1 weight %.

Embodiment 28: The crystalline form of Embodiment 25, wherein the thermogravimetric analysis thermogram is substantially the same as the thermogravimetric analysis thermogram of FIG. 13.

Embodiment 29: The crystalline form of any of Embodiments 3 to 28 further characterized by a gravimetric vapor sorption plot wherein the crystalline form exhibits a reversible moisture uptake of less than about 0.5 weight % from about 0% relative humidity to about 80% relative humidity at 25° C.±0.1° C.

Embodiment 30: The crystalline form of Embodiment 29, wherein the reversible moisture uptake is less than about 0.2 weight %.

Embodiment 31: The crystalline form of Embodiment 29, wherein the reversible moisture uptake is less than about 0.1 weight %.

Embodiment 32: The crystalline form of Embodiment 29, wherein the gravimetric vapor sorption plot is substantially the same as the gravimetric vapor sorption plot of FIG. 14.

Embodiment 33: The crystalline form of any of Embodiments 3 to 10, wherein the crystalline form is further characterized by the following:
a solid-state ¹³C NMR spectrum comprising at least five peaks selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm; and
a differential scanning calorimetry curve comprising a melting endotherm having an onset between about 185° C. to about 200° C.

Embodiment 34: The crystalline form of any of Embodiments 3 to 10, wherein the crystalline form is further characterized by the following:
a solid-state ¹³C NMR spectrum comprising peaks at 166.9±0.2 ppm, 118.6±0.2 ppm, 110.0±0.2 ppm, 49.9±0.2 ppm, and 46.6±0.2 ppm:
a differential scanning calorimetry curve comprising a melting endotherm having an onset between about 185° C. to about 200° C.; and
a thermogravimetric analysis thermogram wherein the crystalline form exhibits a weight loss of less than about 0.2 weight % from about 25° C. to about 110° C.

Embodiment 35: The crystalline form of any of Embodiments 3 to 10, wherein the crystalline form is further characterized by the following:
a solid-state ¹³C NMR spectrum comprising peaks at 166.9±0.2 ppm, 118.6±0.2 ppm, 110.0±0.2 ppm, 49.9±0.2 ppm, and 46.6±0.2 ppm:
a differential scanning calorimetry curve comprising a melting endotherm having an onset between about 185° C. to about 200° C.; and
a gravimetric vapor sorption plot wherein the crystalline form exhibits a reversible moisture uptake of less than about 0.2 weight % from about 0% relative humidity to about 80% relative humidity at 25° C.±0.1° C.

Embodiment 36: The crystalline form of any of Embodiments 3 to 10, wherein the crystalline form is further characterized by the following:
a solid-state ¹³C NMR spectrum comprising peaks at 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm:
a differential scanning calorimetry curve comprising a melting endotherm having an onset at 191° C.±5° C.:
a thermogravimetric analysis thermogram wherein the crystalline form exhibits a weight loss of less than about 0.1 weight % from about 25° C. to about 110° C.; and
a gravimetric vapor sorption plot wherein the crystalline form exhibits a reversible moisture uptake of less than about 0.1 weight % from about 0% relative humidity to about 80% relative humidity at 25° C.±0.1° C.

Embodiment 37: The crystalline form of any of Embodiments 3 to 36, wherein the crystalline form is a crystalline anhydrate.

Embodiment 38: The crystalline form of any of Embodiments 3 to 37, wherein the crystalline form comprises less than 5 weight % of any other crystalline form of (R)—N-(2-(4-cyanothiazolidin-3-yl)-2-oxoethyl)-6-morpholinoquinoline-4-carboxamide.

Embodiment 39: The crystalline form of any of Embodiments 3 to 37, wherein the crystalline form is substantially free of any other crystalline form of (R)—N-(2-(4-cyanothiazolidin-3-yl)-2-oxoethyl)-6-morpholinoquinoline-4-carboxamide.

Embodiment 40: The crystalline form of Embodiment 2, wherein:
the transmission X-ray powder diffraction pattern comprises at least one peak selected from the group consisting of 12.0±0.2° 2θ, 18.4±0.2° 2θ, 19.8±0.2° 2θ, and 21.7° 2θ±0.2° 2θ; and
the transmission X-ray powder diffraction pattern does not comprise peaks at 18.7±0.2° 2θ and 22.4° 2θ±0.2° 2θ having a medium or stronger relative intensity.

Embodiment 41: The crystalline form of Embodiment 40, wherein the transmission X-ray powder diffraction pattern does not comprise peaks at 18.7±0.2° 2θ and 22.4° 2θ±0.2° 2θ.

Embodiment 42: The crystalline form of Embodiment 40, wherein the transmission X-ray powder diffraction pattern further comprises at least two peaks selected from the group consisting of 12.0±0.2° 2θ, 18.4±0.2° 2θ, 19.8±0.2° 2θ, and 21.7° 2θ±0.2° 2θ.

Embodiment 43: The crystalline form of Embodiment 40, wherein the transmission X-ray powder diffraction pattern further comprises peaks at 12.0±0.2° 2θ, 18.4±0.2° 2θ, 19.8±0.2° 2θ, and 21.7° 2θ±0.2° 2θ.

Embodiment 44: The crystalline form of Embodiment 43, wherein the transmission X-ray powder diffraction pattern further comprises at least one peak selected from the group consisting of 13.1±0.2° 2θ, 14.4±0.2° 2θ, 17.5±0.2° 2θ, and 21.1° 2θ±0.2° 2θ.

Embodiment 45: The crystalline form of Embodiment 43, wherein the transmission X-ray powder diffraction pattern further comprises peaks at 13.1±0.2° 2θ, 14.4±0.2° 2θ, 17.5±0.2° 2θ, and 21.1° 2θ±0.2° 2θ.

Embodiment 46: The crystalline form of any of Embodiments 40 to 45, wherein the transmission x-ray powder diffraction is carried out using Cu radiation.

Embodiment 47: The crystalline form of any of Embodiments 40 to 46, wherein the transmission x-ray powder diffraction is carried out using a PANalytical Empyrean diffractometer operating in transmission geometry, a tube voltage of 45 kV, and filament emission of 40 mA.

Embodiment 48: The crystalline form of Embodiment 2 or any of Embodiments 40 to 47 further characterized by a solid-state ¹³C NMR spectrum comprising at least one peak selected from the group consisting of 166.1±0.2 ppm, 130.7±0.2 ppm, 117.9±0.2 ppm, 108.6±0.2 ppm, and 35.2±0.2 ppm.

Embodiment 49: The crystalline form of Embodiment 48, wherein the solid-state ¹³C NMR spectrum comprises peaks at 166.1±0.2 ppm, 117.9±0.2 ppm, and 108.6±0.2 ppm.

Embodiment 50: The crystalline form of Embodiment 48, wherein the solid-state ¹³C NMR spectrum comprises peaks at 166.1±0.2 ppm, 130.7±0.2 ppm, 117.9±0.2 ppm, 108.6±0.2 ppm, and 35.2±0.2 ppm.

Embodiment 51: The crystalline form of Embodiment 48, wherein the solid-state ¹³C NMR spectrum comprises peaks at 168.0±0.2 ppm, 166.1±0.2 ppm, 147.5±0.2 ppm, 146.4±0.2 ppm, 143.1±0.2 ppm, 139.5±0.2 ppm, 130.7±0.2 ppm, 125±0.2 ppm, 117.9±0.2 ppm, 108.6±0.2 ppm, 67.1±0.2 ppm, 48.0±0.2 ppm, 42.9±0.2 ppm, and 35.2±0.2 ppm.

Embodiment 52: The crystalline form of any of Embodiments 48 to 51, wherein the solid-state ¹³C NMR spectrum does not comprise at least one peak selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

Embodiment 53: The crystalline form of any of Embodiments 48 to 51, wherein the solid-state ¹³C NMR spectrum does not comprise at least five peaks selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

Embodiment 54: The crystalline form of any of Embodiments 48 to 51, wherein the solid-state ¹³C NMR spectrum does not comprise peaks at 49.9±0.2 ppm and 46.6±0.2 ppm.

Embodiment 55: The crystalline form of any of Embodiments 48 to 51, wherein the solid-state ¹³C NMR spectrum does not comprise peaks at 166.9±0.2 ppm, 118.6±0.2 ppm, 110.0±0.2 ppm, 49.9±0.2 ppm, and 46.6±0.2 ppm.

Embodiment 56: The crystalline form of any of Embodiments 48 to 51, wherein the solid-state ¹³C NMR spectrum does not comprise peaks at 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

Embodiment 57: The crystalline form of Embodiment 2 or any of Embodiments 40 to 56 further characterized by a differential scanning calorimetry curve comprising a melting endotherm having an onset temperature between about 165° C. to about 180° C.

Embodiment 58: The crystalline form of Embodiment 57, wherein the endotherm comprises a melting endotherm having an onset temperature between about 165° C. to about 177° C.

Embodiment 59: The crystalline form of Embodiment 57, wherein the endotherm has an onset at 171° C.±5° C.

Embodiment 60: The crystalline form of Embodiment 57, wherein the differential scanning calorimetry curve is substantially the same as the differential scanning calorimetry curve of FIG. 6.

Embodiment 61: The crystalline form of any of Embodiments 57 to 60, wherein the differential scanning calorimetry is conducted on a TA Instruments Differential Scanning calorimeter, model Q2000, with a sample placed in an aluminum pan and heated under nitrogen at a rate of 10° C./minute to a temperature of 230° C.

Embodiment 62: The crystalline form of any of Embodiments 40 to 61 further characterized by a thermogravimetric analysis thermogram wherein the crystalline form exhibits a weight loss of less than about 0.5 weight % from about 25° C. to about 110° C.

Embodiment 63: The crystalline form of Embodiment 62, wherein the weight loss is less than about 0.2 weight %.

Embodiment 64: The crystalline form of Embodiment 62, wherein the weight loss is less than about 0.1 weight %.

Embodiment 65: The crystalline form of Embodiment 62, wherein the thermogravimetric analysis thermogram is substantially the same as the thermogravimetric analysis thermogram of FIG. 7.

Embodiment 66: The crystalline form of any of Embodiments 40 to 65 further characterized by a gravimetric vapor sorption plot wherein the crystalline form exhibits a reversible moisture uptake of less than about 0.5 weight % from about 0% relative humidity to about 80% relative humidity at 25° C.±0.1° C.

Embodiment 67: The crystalline form of Embodiment 66, wherein the reversible moisture uptake is less than about 0.2 weight %.

Embodiment 68: The crystalline form of Embodiment 66, wherein the reversible moisture uptake is less than about 0.1 weight %.

Embodiment 69: The crystalline form of Embodiment 66, wherein the gravimetric vapor sorption plot is substantially the same as the gravimetric vapor sorption plot of FIG. 8.

Embodiment 70: The crystalline form of any of Embodiments 40 to 47, wherein the crystalline form is further characterized by the following:
a solid-state ¹³C NMR spectrum comprising peaks at 166.1±0.2 ppm, 130.7±0.2 ppm, 117.9±0.2 ppm, 108.6±0.2 ppm, and 35.2±0.2 ppm; and
a differential scanning calorimetry curve comprising a melting endotherm having an onset temperature between about 165° C. to about 180° C.

Embodiment 71: The crystalline form of any of Embodiments 40 to 47, wherein the crystalline form is further characterized by the following:
a solid-state ¹³C NMR spectrum comprising peaks at 166.1±0.2 ppm, 130.7±0.2 ppm, 117.9±0.2 ppm, 108.6±0.2 ppm, and 35.2±0.2 ppm:
a differential scanning calorimetry curve comprising a melting endotherm having an onset temperature between about 165° C. to about 177° C.; and
a thermogravimetric analysis thermogram wherein the crystalline form exhibits a weight loss of less than about 0.2 weight % from about 25° C. to about 110° C.

Embodiment 72: The crystalline form of any of Embodiments 40 to 47, wherein the crystalline form is further characterized by the following:
a solid-state ¹³C NMR spectrum comprising peaks at 166.1±0.2 ppm, 130.7±0.2 ppm, 117.9±0.2 ppm, 108.6±0.2 ppm, and 35.2±0.2 ppm:
a differential scanning calorimetry curve comprising a melting endotherm having an onset temperature between about 165° C. to about 177° C.; and
a gravimetric vapor sorption plot wherein the crystalline form exhibits a reversible moisture uptake of less than about 0.2 weight % from about 0% relative humidity to about 80% relative humidity at 25° C.±0.1° C.

Embodiment 73: The crystalline form of any of Embodiments 40 to 47, wherein the crystalline form is further characterized by the following:
a solid-state ¹³C NMR spectrum comprising peaks at 166.1±0.2 ppm, 130.7±0.2 ppm, 117.9±0.2 ppm, 108.6±0.2 ppm, and 35.2±0.2 ppm;
a differential scanning calorimetry curve comprising a melting endotherm having an onset at 171° C.±5° C.:
a thermogravimetric analysis thermogram wherein the crystalline form exhibits a weight loss of less than about 0.2 weight % from about 25° C. to about 110° C.; and
a gravimetric vapor sorption plot wherein the crystalline form exhibits a reversible moisture uptake of less than about 0.2 weight % from about 0% relative humidity to about 80% relative humidity at 25° C.±0.1° C.

Embodiment 74: The crystalline form of any of Embodiments 40 to 73, wherein the crystalline form is a crystalline anhydrate.

Embodiment 75: The crystalline form of any of Embodiments 40 to 74, wherein the crystalline form comprises less than 5 weight % of any other crystalline form of (R)—N-(2-(4-cyanothiazolidin-3-yl)-2-oxoethyl)-6-morpholinoquinoline-4-carboxamide.

Embodiment 76: The crystalline form of any of Embodiments 40 to 74, wherein the crystalline form is substantially free of any other crystalline form of (R)—N-(2-(4-cyanothiazolidin-3-yl)-2-oxoethyl)-6-morpholinoquinoline-4-carboxamide.

Embodiment 77: The crystalline form of Embodiment 1 that is a crystalline form of (R,S)—N-(2-(4-cyanothiazolidin-3-yl)-2-oxoethyl)-6-morpholinoquinoline-4-carboxamide.

Embodiment 78: The crystalline form of Embodiment 77 characterized by a reflection X-ray powder diffraction pattern comprising at least one peak selected from the group consisting of 10.0±0.2° 2θ, 12.9±0.2° 2θ, 17.1±0.2° 2θ, 22.0±0.2° 2θ, and 22.8° 2θ±0.2° 2θ.

Embodiment 79: The crystalline form of Embodiment 78, wherein the reflection X-ray powder diffraction pattern comprises peaks at 10.0±0.2° 2θ, 12.9±0.2° 2θ, 17.1±0.2° 2θ, 22.0±0.2° 2θ, and 22.8° 2θ±0.2° 2θ.

Embodiment 80: The crystalline form of Embodiment 79, wherein the reflection X-ray powder diffraction pattern further comprises at least one peak selected from the group consisting of 15.8±0.2° 2θ, 16.2±0.2° 2θ, 26.0±0.2° 2θ, 26.5±0.2° 2θ, and 26.9° 2θ±0.2° 2θ.

Embodiment 81: The crystalline form of Embodiment 79, wherein the reflection X-ray powder diffraction pattern further comprises peaks at 15.8±0.2° 2θ, 16.2±0.2° 2θ, 26.0±0.2° 2θ, 26.5±0.2° 2θ, and 26.9° 2θ±0.2° 2θ.

Embodiment 82: The crystalline form of any of Embodiments 77 to 81, wherein the reflection x-ray powder diffraction is carried out using Cu radiation.

Embodiment 83: The crystalline form of any of Embodiments 77 to 82, wherein the reflection x-ray powder diffraction is carried out using a PANalytical Empyrean diffractometer operating in reflection geometry, a tube voltage of 45 kV, and filament emission of 40 mA.

Embodiment 84: The crystalline form of any of Embodiments 77 to 83 further characterized by a differential scanning calorimetry curve comprising a melting endotherm between about 195° C. to about 210° C.

Embodiment 85: The crystalline form of Embodiment 84, wherein the endotherm has an onset temperature between about 195° C. to about 207° C.

Embodiment 86: The crystalline form of Embodiment 84, wherein the endotherm has an onset temperature at 201° C.±5° C.

Embodiment 87: The crystalline form of Embodiment 85, wherein the differential scanning calorimetry curve is substantially the same as the differential scanning calorimetry curve of FIG. 16.

Embodiment 88: The crystalline form of any of Embodiments 84 to 87, wherein the differential scanning calorimetry is conducted on a TA Instruments Differential Scanning calorimeter, model Q2000, with a sample placed in an aluminum pan and heated under nitrogen at a rate of 10° C./minute to a temperature of 230° C.

Embodiment 89: The crystalline form of any of Embodiments 77 to 88 further characterized by a gravimetric vapor sorption plot wherein the crystalline form exhibits a reversible moisture uptake of less than about 1.0 weight % from about 0% relative humidity to about 80% relative humidity at 25° C.±0.1° C.

Embodiment 90: The crystalline form of Embodiment 89, wherein the reversible moisture uptake is less than about 0.7 weight %.

Embodiment 91: The crystalline form of Embodiment 89, wherein the reversible moisture uptake is about 0.5 weight %.

Embodiment 92: The crystalline form of Embodiment 89, wherein the gravimetric vapor sorption plot is substantially the same as the gravimetric vapor sorption plot of FIG. 17.

Embodiment 93: The crystalline form of any of Embodiments 78 to 92, wherein the crystalline form is further characterized by the following:
a differential scanning calorimetry curve comprising a melting endotherm having an onset temperature between about 190° C. to about 210° C.; and
a gravimetric vapor sorption plot wherein the crystalline form exhibits a reversible moisture uptake of less than about 1.0 weight % from about 0% relative humidity to about 80% relative humidity at 25° C.±0.1° C.

Embodiment 94: The crystalline form of any of Embodiments 78 to 92, wherein the crystalline form is further characterized by the following:
a differential scanning calorimetry curve comprising a melting endotherm having an onset temperature between about 195° C. to about 207° C.; and
a gravimetric vapor sorption plot wherein the crystalline form exhibits a reversible moisture uptake of less than about 0.7 weight % from about 0% relative humidity to about 80% relative humidity at 25° C.±0.1° C.

Embodiment 95: The crystalline form of any of Embodiments 78 to 92, wherein the crystalline form is further characterized by the following:
a differential scanning calorimetry curve comprising a melting endotherm having an onset at 201° C.±5° C.; and
a gravimetric vapor sorption plot wherein the crystalline form exhibits a reversible moisture uptake of about 0.5 weight % from about 0% relative humidity to about 80% relative humidity at 25° C.±0.1° C.

Embodiment 96: The crystalline form of any of Embodiments 78 to 95, wherein the crystalline form is a crystalline anhydrate.

Embodiment 97: The crystalline form of any of Embodiments 78 to 96, wherein the crystalline form comprises less than 5 weight % of any other crystalline form of N-(2-(4-cyanothiazolidin-3-yl)-2-oxoethyl)-6-morpholinoquinoline-4-carboxamide.

Embodiment 98: The crystalline form of any of Embodiments 78 to 96, wherein the crystalline form is substantially free of any other crystalline form of N-(2-(4-cyanothiazolidin-3-yl)-2-oxoethyl)-6-morpholinoquinoline-4-carboxamide.

Embodiment 99: A composition comprising at least two crystalline forms selected from the group consisting of:
the crystalline form of any of Embodiments 3 to 37:
the crystalline form of any of Embodiments 40 to 74; and
the crystalline form of any of Embodiments 77 to 96.

Embodiment 100: The composition of Embodiment 99, wherein the composition comprises:
the crystalline form of any of Embodiments 3 to 37; and
the crystalline form of any of Embodiments 40 to 74.

Embodiment 101: The composition of Embodiment 100, wherein the composition comprises at least 50 weight % of the crystalline form of any of Embodiments 3 to 37 relative to the crystalline form of any of Embodiments 40 to 74.

Embodiment 102: The composition of Embodiment 100, wherein the composition comprises at least 75 weight % of the crystalline form of any of Embodiments 3 to 37 relative to the crystalline form of any of Embodiments 40 to 74.

Embodiment 103: The composition of Embodiment 100, wherein the composition comprises at least 90 weight % of the crystalline form of any of Embodiments 3 to 37 relative to the crystalline form of any of Embodiments 40 to 74.

Embodiment 104: The composition of Embodiment 100, wherein the composition comprises at least 95 weight % of the crystalline form of any of Embodiments 3 to 37 relative to the crystalline form of any of Embodiments 40 to 74.

Embodiment 105: The composition of Embodiment 100, wherein the composition comprises at least 95 weight % of the crystalline form of any of Embodiments 3 to 37 relative to any other crystalline form.

Embodiment 106: A pharmaceutical composition comprising the crystalline form of any of Embodiments 1 to 98, and one or more pharmaceutically acceptable excipients.

Embodiment 107: The pharmaceutical composition of Embodiment 106, wherein the pharmaceutical composition comprises the crystalline form of any of Embodiments 3 to 37.

Embodiment 108: The pharmaceutical composition of Embodiment 107, wherein the pharmaceutical composition further comprises the crystalline form of any of Embodiments 40 to 74.

Embodiment 109: The pharmaceutical composition of Embodiment 106, wherein the pharmaceutical composition comprises the crystalline form of any of Embodiments 40 to 70.

Embodiment 110: The pharmaceutical composition of any of Embodiments 106 to 109, wherein the pharmaceutical composition is a solid pharmaceutical composition.

Embodiment 111: A method of treating or preventing an FAP-mediated condition in a subject suffering from or susceptible to the FAP-mediated condition, the method comprising administering to the subject a therapeutically effective amount of a crystalline form of any of Embodiments 1 to 98.

Embodiment 112: The method of Embodiment 111, wherein the FAP-mediated condition is selected from the group consisting of liver disease, type 2 diabetes mellitus, cardiovascular conditions, obesity, obesity-related conditions, fibrosis, keloid disorder, inflammation, and cancer.

Embodiment 113: The method of Embodiment 112, wherein the FAP-mediated condition is liver disease.

Embodiment 114: The method of Embodiment 113, wherein the liver disease is nonalcoholic steatohepatitis.

Embodiment 115: The use of a compound of any of Embodiments 1 to 98, or a pharmaceutically acceptable salt thereof, for the manufacture of a medicament for treating or preventing an FAP-mediated condition.

### III. Methods of Use

(R)—N-(2-(4-cyanothiazolidin-3-yl)-2-oxoethyl)-6-morpholinoquinoline-4-carboxamide is an inhibitor of Prolyl endopeptidase fibroblast activation protein (FAP) activity. FAP is an endopeptidase that enzymatically cleaves substrates involved in glucose and lipid metabolism, fibrinolysis, and collagen production.

FAP is believed to cleave and inactivate human Fibroblast Growth Factor 21 (FGF-21) (Biochem. J. 2016, 473, 605), a protein involved in the regulation of glucose and lipid metabolism. It is hypothesized that inhibition of FAP increases endogenous FGF-21 levels and signaling, and results, for example, in decreased steatosis, improved insulin sensitivity, improved glucose tolerance, reduced body weight, and/or reduced cardiovascular disease mortality.

FAP is also believed to cleave human α2-Antiplasmin (α2AP) (Blood 2004 103, 3783), a protein involved in the regulation of fibrosis and fibrinolysis. Tissue repair involves coagulation which results in fibrin deposition. The fibrin of a clot is usually lysed, primarily by plasmin when converted from its inactive form (plasminogen) by plasminogen activators. Fibrinolysis is inhibited by Plasminogen Activator Inhibitor-1 (PAI-1), Plasminogen Activator Inhibitor-2 (PAI-2), and α2AP, (Experimental & Molecular Medicine 2020, 52, 367) all of which are induced by tissue trauma. FAP converts α2AP into a form more effectively bound to fibrin, which reduces plasmin degradation of fibrin at the site of an injury. It is hypothesized that inhibition of FAP increases fibrinolysis and improves tissue regeneration at the site of injury (J. Thromb. Haemost. 2013, 11, 2029; Proteomics Clin. Appl. 2014, 8, 454).

FAP is further believed to promote collagen production and deposition and to play a role in increased fibrosis through altered extracellular matrix (ECM) turnover (J Biol Chem 2016, 8, 291). It is hypothesized that inhibition of FAP results in a decrease in collagen deposition and a reduction in inflammation (Inflamm. Bowel Dis. 2018, 18, 332).

In view of the above, it is hypothesized that inhibition of FAP collectively reduces fibrosis and inflammation by decreasing hepatic stellate cell activity and increasing fibrinolysis, and further provides positive metabolic effects through increased FGF21 signaling and improved glucose tolerance.

In some embodiments, therefore, the present disclosure provides a method for treating or preventing an FAP-mediated condition in a subject in need thereof by administering to the subject a pharmaceutical composition comprising a therapeutically effective amount of a crystalline form of the present disclosure.

In some embodiments, the present disclosure provides a method for treating or preventing a condition characterized by overexpression of FAP in a subject in need thereof by administering to the subject a pharmaceutical composition comprising a therapeutically effective amount of a crystalline form of the present disclosure.

In some embodiments, the present disclosure provides a method for treating or preventing liver disease in a subject in need thereof by administering to the subject a pharmaceutical composition comprising a therapeutically effective amount of a crystalline form of the present disclosure. In one aspect, the liver disease is a fatty liver disease. In another aspect, the liver disease is Nonalcoholic Fatty Liver Disease (NAFLD). In another aspect, the NAFLD is selected from the group consisting of isolated steatosis, Nonalcoholic Steatohepatitis (NASH), liver fibrosis, and cirrhosis. In another aspect, the liver disease is end stage liver disease. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating liver disease in a subject in need thereof by administering to the subject a pharmaceutical composition comprising a therapeutically effective amount of a crystalline form of the present disclosure, wherein the subject has a body mass index (BMI) of 27 kg/m² to 40 kg/m². In one aspect, the subject has a BMI of 30 kg/m² to 39.9 kg/m². In another aspect, the subject has a BMI of at least 40 kg/m². In another aspect, the subject is overweight. In another aspect, the subject is obese. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a pharmaceutical composition comprising a therapeutically effective amount of a crystalline form of the present disclosure, wherein the subject is also suffering from or susceptible to dyslipidemia. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a pharmaceutical composition comprising a therapeutically effective amount of a crystalline form of the present disclosure, wherein the subject is also suffering from or susceptible to insulin resistance. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a pharmaceutical composition comprising a therapeutically effective amount of a crystalline form of the present disclosure, wherein the subject is also suffering from or susceptible to at least one of Type 2 diabetes and renal insufficiency. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a pharmaceutical composition comprising a therapeutically effective amount of a crystalline form of the present disclosure, wherein the subject is also suffering from or susceptible to Type 2 diabetes. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for treating liver disease in a subject in need thereof by administering to the subject a pharmaceutical composition comprising a therapeutically effective amount of a crystalline form of the present disclosure, wherein the subject is also suffering from or susceptible to renal insufficiency. In another aspect, the liver disease is NAFLD. In another aspect, the liver disease is NASH. In another aspect, the liver disease is liver fibrosis. In another aspect, the liver disease is cirrhosis.

In some embodiments, the present disclosure provides a method for reducing liver fat in a subject in need thereof by administering to the subject a pharmaceutical composition comprising a therapeutically effective amount of a crystalline form of the present disclosure. In one aspect, the subject is suffering from or susceptible to NAFLD. In another aspect, the subject is suffering from or susceptible to NASH. In another aspect, the subject is suffering from or susceptible to liver fibrosis. In another aspect, the subject is suffering from or susceptible to cirrhosis. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing Nonalcoholic Fatty Liver Disease (NAFLD) in a subject in need thereof by administering to the subject a pharmaceutical composition comprising a therapeutically effective amount of a crystalline form of the present disclosure. In one aspect, the NAFLD is Stage 1 NAFLD. In another aspect, the NAFLD is Stage 2 NAFLD. In another aspect, the NAFLD is Stage 3 NAFLD. In another aspect, the NAFLD is Stage 4 NAFLD. See, e.g., “The Diagnosis and Management of Nonalcoholic Fatty Liver Disease: Practice Guidance From the American Association for the Study of Liver Diseases,” Hepatology, 2018, Vol. 67, No. 1. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing Nonalcoholic Steatohepatitis (NASH) in a subject in need thereof by administering to the subject a pharmaceutical composition comprising a therapeutically effective amount of a crystalline form of the present disclosure. In one aspect, the NASH is Stage 1 NASH. In another aspect, the NASH is Stage 2 NASH. In another aspect, the NASH is Stage 3 NASH. In another aspect, the NASH is Stage 4 NASH. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing liver fibrosis in a subject in need thereof by administering to the subject a pharmaceutical composition comprising a therapeutically effective amount of a crystalline form of the present disclosure. In one aspect, the subject is suffering from Stage 3 liver fibrosis. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing cirrhosis in a subject in need thereof by administering to the subject a pharmaceutical composition comprising a therapeutically effective amount of a crystalline form of the present disclosure. In one aspect, the subject is suffering from stage F4 cirrhosis. In another aspect, the subject is also suffering from or susceptible to one or more conditions selected from the group consisting of obesity, dyslipidemia, insulin resistance, Type 2 diabetes, and renal insufficiency.

In some embodiments, the present disclosure provides a method for treating or preventing type 2 diabetes mellitus in a subject in need thereof by administering to the subject a pharmaceutical composition comprising a therapeutically effective amount of a crystalline form of the present disclosure. In one aspect, the subject is suffering from diabetic kidney disease. In another aspect, the subject is suffering from renal insufficiency. In another aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese.

In some embodiments, the present disclosure provides a method of improving glycemic control in a subject in need thereof by administering to the subject a pharmaceutical composition comprising a therapeutically effective amount of a crystalline form of the present disclosure. In one aspect, the subject is suffering from type 2 diabetes.

In another aspect, the subject suffering from diabetic kidney disease. In another aspect, the subject is suffering from renal insufficiency. In another aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese.

In some embodiments, the present disclosure provides a method of improving glycemic control in a subject with type 2 diabetes and diabetic kidney disease by administering to the subject a pharmaceutical composition comprising a therapeutically effective amount of a crystalline form of the present disclosure. In one aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese.

In some embodiments, the present disclosure provides a method of improving glycemic control in a subject with type 2 diabetes and renal insufficiency by administering to the subject a pharmaceutical composition comprising a therapeutically effective amount of a crystalline form of the present disclosure. In one aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese.

In some embodiments, the present disclosure provides a method of treating or preventing insulin resistance in a subject thereof by administering to the subject a pharmaceutical composition comprising a therapeutically effective amount of a crystalline form of the present disclosure. In another aspect, the subject is suffering from type 2 diabetes. In another aspect, the subject is suffering from diabetic kidney disease. In another aspect, the subject is suffering from renal insufficiency. Insulin resistance can be measured, for example, using the Homeostatic Model Assessment of Insulin Resistance (HOMA-IR) and/or the MATSUDA index. The HOMA-IR is explained, for example, in Diabetologia 1985, 28, 412, which is herein incorporated by reference in its entirety. The MATSUDA index is explained, for example, in Diabetes Care 1999, 22, 1462, which is herein incorporated by reference in its entirety.

In some embodiments, the present disclosure provides a method of treating or preventing glucose intolerance in a subject in need thereof by administering to the subject a pharmaceutical composition comprising a therapeutically effective amount of a crystalline form of the present disclosure. In one aspect, the subject is suffering from type 2 diabetes. In another aspect, the subject is suffering from diabetic kidney disease. In another aspect, the subject is suffering from renal insufficiency.

In some embodiments, the present disclosure provides a method of treating a cardiovascular condition in a subject in need of treatment by administering to the subject a pharmaceutical composition comprising a therapeutically effective amount of a crystalline form of the present disclosure. In one aspect, the cardiovascular condition is selected from the group consisting of heart failure, cardiomyopathy, atherosclerosis, venous thromboembolism, and atrial fibrillation. In one aspect, the cardiovascular condition is heart failure. In another aspect, the cardiovascular condition is heart failure with preserved ejection fraction (HFpEF). In another aspect, the cardiovascular condition is cardiomyopathy. In another aspect, the cardiomyopathy is selected from the group consisting of hypertrophic cardiomyopathy, dilated cardiomyopathy, restrictive cardiomyopathy, hypertrophic cardiomyopathy, ischemic cardiomyopathy, ischemic cardiomyopathy, dilated cardiomyopathy, and idiopathic cardiomyopathy. In another aspect, the cardiovascular condition is atherosclerosis. In another aspect, the cardiovascular condition is venous thromboembolism. In another aspect, the cardiovascular condition is atrial fibrillation.

In some embodiments, the present disclosure provides a method of treating obesity or an obesity-related condition in a subject in need thereof by administering to the subject a pharmaceutical composition comprising a therapeutically effective amount of a crystalline form of the present disclosure. In one aspect, the obesity-related condition is an obesity-related metabolic condition. In another aspect, the obesity-related condition is selected from the group consisting of insulin resistance, pre-diabetes, type 2 diabetes, glucose intolerance, increased fasting glucose, and glucagonomas. In another aspect, the obesity-related condition is dyslipidemia. In another aspect, the obesity-related condition is a cardiovascular condition is selected from the group consisting of heart failure, cardiomyopathy, atherosclerosis, venous thromboembolism, and atrial fibrillation. In another aspect, the obesity-related condition is renal disease.

In some embodiments, the present disclosure provides a method of reducing body weight in a subject in need thereof by administering to the subject a pharmaceutical composition comprising a therapeutically effective amount of a crystalline form of the present disclosure. In one aspect, the subject is suffering from type 2 diabetes. In another aspect, the subject is suffering from diabetic kidney disease. In another aspect, the subject is suffering from renal insufficiency. In another aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese. In another aspect, the subject's weight is reduced, for example, by at least about 5%, 10%, 15%, 20%, 25%, 30%, 35%, or 40%.

In some embodiments, the present disclosure provides a method of reducing body fat in a subject in need of treatment by administering to the subject a pharmaceutical composition comprising a therapeutically effective amount of a crystalline form of the present disclosure. In another aspect, the subject is suffering from type 2 diabetes. In another aspect, the subject is suffering from diabetic kidney disease. In another aspect, the subject is suffering from renal insufficiency. In another aspect, the administration of the compound is an adjunct to diet and exercise. In another aspect, the administration of the compound also reduces body weight and/or treats obesity. In another aspect, the subject has a BMI of 27 kg/m² to 40 kg/m². In another aspect, the subject has a BMI of 30 kg/m² to 39.9 kg/m². In another aspect, the subject has a BMI of at least 40 kg/m². In another aspect, the subject is overweight. In another aspect, the subject is obese. In another aspect, the fat is liver fat.

In some embodiments, the present disclosure provides a method for treating or preventing fibrosis in a subject in need thereof by administering to the subject a pharmaceutical composition comprising a therapeutically effective amount of a crystalline form of the present disclosure. In one aspect, the fibrosis is interstitial lung disease. In another aspect, the fibrosis is interstitial lung disease with progressive fibrosis. In another aspect, the interstitial lung disease is pulmonary fibrosis. In another aspect, the interstitial lung disease is idiopathic pulmonary fibrosis (IPF).

In some embodiments, the present disclosure provides a method for promoting tissue remodeling in a subject in need thereof by administering to the subject a pharmaceutical composition comprising a therapeutically effective amount of a crystalline form of the present disclosure. In one aspect, the subject has suffered cardiac tissue damage due to a myocardial infarction.

In some embodiments, the present disclosure provides a method of promoting wound healing and/or reducing adhesions in a subject in need thereof by administering to the subject a pharmaceutical composition comprising a therapeutically effective amount of a crystalline form of the present disclosure. In one aspect, the administration of the crystalline form promotes wound healing and/or reduces adhesions through increased fibrinolysis.

In some embodiments, the present disclosure provides a method for treating or preventing a keloid disorder in a subject in need thereof by administering to the subject a pharmaceutical composition comprising a therapeutically effective amount of a crystalline form of the present disclosure. In one aspect, the keloid disorder is selected from the group consisting of scar formation, keloid tumors, and keloid scar.

In some embodiments, the present disclosure provides a method for treating or preventing inflammation in a subject in need thereof by administering to the subject a pharmaceutical composition comprising a therapeutically effective amount of a crystalline form of the present disclosure. In one aspect, the inflammation is chronic inflammation. In one aspect, the chronic inflammation is selected from the group consisting of rheumatoid arthritis, osteoarthritis, and Crohn's disease. In another aspect, the chronic inflammation is rheumatoid arthritis.

In some embodiments, the present disclosure provides a method of treating cancer in a subject in need of treatment by administering to the subject a pharmaceutical composition comprising a therapeutically effective amount of a crystalline form of the present disclosure. In one aspect, the cancer is selected from the group consisting of breast cancer, pancreatic cancer, small intestine cancer, colon cancer, rectal cancer, lung cancer, head and neck cancer, ovarian cancer, hepatocellular carcinoma, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma, and prostate cancer. In another aspect, the cancer is hepatocellular carcinoma.

The subject treated typically will be a human or non-human mammal, particularly a human. Suitable subjects can also include domestic or wild animals; companion animals (including dogs, cats, and the like); livestock (including horses, cows and other ruminants, pigs, poultry, rabbits, and the like); primates (including monkeys such as rhesus monkeys, cynomolgus (also known as crab-eating or long-tailed) monkeys, marmosets, tamarins, chimpanzees, macaques, and the like); and rodents (including rats, mice, gerbils, guinea pigs, and the like).

In some embodiments, the present disclosure provides the crystalline forms of the present disclosure for use as medicaments.

In some embodiments, the present disclosure provides for the use of the crystalline forms of the present disclosure for treating or preventing an FAP-mediated condition as discussed above.

In some embodiments, the present disclosure provides for the use of the crystalline forms of the present disclosure for the manufacture of medicaments for treating or preventing an FAP-mediated condition as discussed above.

### IV. Combination Therapies and Fixed-Dose Combinations

The crystalline forms of the present disclosure may be used in the methods described above as either as single pharmacological agents or in combination with other pharmacological agents or techniques. Such combination therapies may be achieved by way of the simultaneous, sequential or separate dosing of the individual components of the treatment. These combination therapies (and corresponding combination products) employ the crystalline forms of the present disclosure within the dosage ranges described in this specification and the other pharmacological agent(s), typically within its approved dosage range(s).

In some embodiments, the present disclosure provides a combination suitable for use in the treatment of a condition selected from the previously discussed conditions, wherein the combination comprises a crystalline form of the present disclosure and a sodium-glucose transport protein 2 (SGLT2) inhibitor. In one aspect, the SGLT2 inhibitor is selected from the group consisting of canagliflozin, dapagliflozin, empagliflozin, ertugliflozin, ipragliflozin, luseogliflozin, and remogliflozin. In another aspect, the SGLT2 inhibitor is dapagliflozin.

In some embodiments, the present disclosure provides a combination suitable for use in the treatment of a condition selected from the previously discussed conditions, wherein the combination comprises a crystalline form of the present disclosure and metformin.

In some embodiments, the present disclosure provides a combination suitable for use in the treatment of a condition selected from the previously discussed conditions, wherein the combination comprises a crystalline form of the present disclosure and a glucagon-like peptide-1 receptor (GLP1) agonist. In one aspect, the GLP1 agonist is selected from the group consisting of exenatide, liraglutide, lixisenatide, albiglutide, dulaglutide, and semaglutide.

In some embodiments, the present disclosure provides a combination suitable for use in the treatment of a condition selected from the previously discussed conditions, wherein the combination comprises a crystalline form of the present disclosure and a dipeptidyl peptidase 4 (DPP4) inhibitor. In one aspect, the DPP4 inhibitor is selected from the group consisting of sitagliptin, vildagliptin, saxagliptin, linagliptin, gemigliptin, anagliptin, teneligliptin, alogliptin, trelagliptin, omarigliptin, evogliptin, gosogliptin, and dutogliptin.

In some embodiments, the present disclosure provides a combination suitable for use in the treatment of a condition selected from the previously discussed conditions, wherein the combination comprises a crystalline form of the present disclosure and a peroxisome proliferator-activated receptor (PPAR) agonist. In one aspect, the PPAR agonist is a PPARα agonist. In another aspect, the PPAR agonist is a PPARγ agonist. In another aspect, the PPAR agonist is a PPARα/γ agonist. In another aspect, the PPAR agonist is selected from the group consisting of clofibrate, gemfibrozil, ciprofibrate, bezafibrate, and fenofibrate. In another aspect, the PPAR agonist is a thiazolidinedione. In another aspect, the thiazolidinedione is selected from the group consisting of pioglitazone, rosiglitazone, lobeglitazone, and rivoglitazone. In another aspect, the PPAR agonist stimulates liver expression of FGF21.

In some embodiments, the present disclosure provides a pharmaceutical composition comprising a crystalline form of the present disclosure; one or more pharmacological agents selected from SGLT2 inhibitors, metformin, GLP1 agonists, DPP4 inhibitors, and PPAR agonists; and a pharmaceutically acceptable diluent or carrier. Such a combination can be used for the manufacture of a medicament for use in the treatment of a condition selected from the previously discussed conditions. In one aspect, the pharmaceutical composition comprises an SGLT2 inhibitor. In another aspect, the pharmaceutical composition comprises metformin. In another aspect, the pharmaceutical composition comprises a GLP1 agonist. In another aspect, the pharmaceutical composition comprises a DPP4 inhibitor. In another aspect, the pharmaceutical composition comprises a PPAR agonist.

In some embodiments, the present disclosure provides a combination suitable for use in the treatment of cancer, wherein the combination comprises a crystalline form of the present disclosure and an immune checkpoint inhibitor. In one aspect, the immune checkpoint inhibitor is selected from the group consisting of anti-PD-1 antibodies, anti-PD-L1 antibodies, anti-CTLA4 antibodies, TLR7 agonists, CD40 agonists, Lag-3 antagonists, and OX40 agonists. In another aspect, the immune checkpoint inhibitor is an anti-PD-1 antibody (e.g., pembrolizumab (Keytruda), nivolumab (Opdivo), cemiplimab (Libtayo), etc.). In another aspect, the immune checkpoint inhibitor is an anti-PD-L1 antibody (e.g., atezolizumab (Tecentriq), avelumab (Bavencio), durvalumab (Imfinzi), etc.). In another aspect, the immune checkpoint inhibitor is an anti-CTLA4 antibody (e.g., ipilimumab (Yervoy), tremelimumab, etc.). In another aspect, the cancer is selected from the group consisting of pancreatic cancer, colon cancer, and rectal cancer.

### V. Pharmaceutical Compositions

The crystalline forms of the present disclosure may be administered as pharmaceutical compositions, comprising one or more pharmaceutically acceptable excipients. Therefore, in some embodiments the present disclosure provides pharmaceutical compositions comprising a crystalline form of the present disclosure, and at least one pharmaceutically acceptable excipient.

The excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth Edition, Pharmaceutical Press, edited by Rowe, Ray C; Sheskey, Paul J; Quinn, Marian. Pharmaceutically acceptable excipients may function as, for example, adjuvants, diluents, carriers, stabilisers, flavourings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

The compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous or intramuscular dosing), or as a suppository for rectal dosing. The compositions may be obtained by conventional procedures using conventional pharmaceutical excipients, well known in the art. Thus, compositions intended for oral use may contain, for example, one or more coloring, sweetening, flavoring and/or preservative agents.

The total daily dose will necessarily be varied depending upon the subject treated, the route of administration, any therapies being co-administered, and the severity of the illness being treated, and may include single or multiple doses. Specific dosages can be adjusted, for example, depending upon the condition being treated; the age, body weight, general health condition, sex, and diet of the subject; administration routes; dose intervals; excretion rate; and other drugs being co-administered to the subject. An ordinarily skilled physician provided with the disclosure of the present specification will be able to determine appropriate dosages and regimens for administration of the therapeutic agent to the subject, and to adjust such dosages and regimens as necessary during the course of treatment, in accordance with methods well-known in the therapeutic arts. The crystalline form of the present disclosure typically will be administered to a warm-blooded animal at a unit dose within the range 2.5 to 5000 mg/m² body area of the animal, or approximately 0.05 to 100 mg/kg, and this normally provides a therapeutically effective dose.

In some embodiments, the present disclosure provides pharmaceutical compositions for use in therapy, comprising a crystalline form of the present disclosure and at least one pharmaceutically acceptable excipient.

In some embodiments, the present disclosure provides pharmaceutical compositions for use in the treatment of an FAP-mediated condition, comprising a crystalline form of the present disclosure and at least one pharmaceutically acceptable excipient. In one aspect, the FAP-mediated condition is selected from the group consisting of liver disease, type 2 diabetes mellitus, cardiovascular conditions, obesity, obesity-related conditions, fibrosis, keloid disorder, inflammation, and cancer.

In some embodiments, the present disclosure provides a pharmaceutical composition comprising Form A, and one or more pharmaceutically acceptable excipients. In one aspect, the composition comprises at least 90 weight % of Form A relative to any other crystalline forms. In another aspect, the composition comprises at least 95 weight % of Form A relative to any other crystalline forms. In another aspect, the composition comprises at least 96 weight % of Form A relative to any other crystalline forms. In another aspect, the composition comprises at least 97 weight % of Form A relative to any other crystalline forms. In another aspect, the composition comprises at least 98 weight % of Form A relative to any other crystalline forms. In another aspect, the composition comprises at least 99 weight % of Form A relative to any other crystalline forms.

In some embodiments, the present disclosure provides a pharmaceutical composition comprising Form B, and one or more pharmaceutically acceptable excipients. In one aspect, the composition comprises at least 90 weight % of Form B relative to any other crystalline forms. In another aspect, the composition comprises at least 95 weight % of Form B relative to any other crystalline forms. In another aspect, the composition comprises at least 96 weight % of Form B relative to any other crystalline forms. In another aspect, the composition comprises at least 97 weight % of Form B relative to any other crystalline forms. In another aspect, the composition comprises at least 98 weight % of Form B relative to any other crystalline forms. In another aspect, the composition comprises at least 99 weight % of Form B relative to any other crystalline forms.

In some embodiments, the present disclosure provides a pharmaceutical composition comprising Form A and Form B, and one or more pharmaceutically acceptable excipients. In one aspect, the composition comprises at least 50 weight % of Form B relative to Form A. In another aspect, the composition comprises at least 75 weight % of Form B relative to Form A. In another aspect, the composition comprises at least 90 weight % of Form B relative to Form A. In another aspect, the composition comprises at least 95 weight % of Form B relative to Form A. In another aspect, the composition comprises at least 99 weight % of Form B relative to Form A.

In some embodiments, the present disclosure provides a pharmaceutical composition comprising Type 2, and one or more pharmaceutically acceptable excipients. In one aspect, the composition comprises at least 90 weight % of Type 2 relative to any other crystalline forms. In another aspect, the composition comprises at least 95 weight % of Type 2 relative to any other crystalline forms. In another aspect, the composition comprises at least 96 weight % of Type 2 relative to any other crystalline forms. In another aspect, the composition comprises at least 97 weight % of Type 2 relative to any other crystalline forms. In another aspect, the composition comprises at least 98 weight % of Type 2 relative to any other crystalline forms. In another aspect, the composition comprises at least 99 weight % of Type 2 relative to any other crystalline forms.

### VI. Kits

The present disclosure further provides kits comprising a unit dosage form comprising a crystalline form of the present disclosure contained within a packaging material and a label or package insert which indicates that the unit dosage form can be used for treating one or more of the previously described conditions.

In some embodiments, the kit comprises a unit dosage form comprising a crystalline form of the present disclosure contained within a packaging material and a label or package insert which indicates that the pharmaceutical composition can be used for treating an FAP-mediated condition. In one aspect, the FAP-mediated condition is liver disease. In another aspect, the liver disease is selected from the group consisting of fatty liver disease, end stage liver disease, and cirrhosis. In another aspect, the liver disease is selected from the group consisting of Nonalcoholic Steatohepatitis (NASH) and Nonalcoholic Fatty Liver Disease (NAFLD).

In some embodiments, the kit comprises: (a) a first unit dosage form comprising a crystalline form of the present disclosure; (b) a second unit dosage form comprising a pharmacological agent selected from the group consisting of SGLT2 inhibitors, metformin, GLP1 agonists, DPP4 inhibitors, and PPAR agonists; (c) a container means for containing said first and second dosage forms; and (d) a label or package insert which indicates that the first unit dosage form and second unit dosage form can be used for treating an FAP-mediated condition.

### VII. Examples

The following descriptions of experiments, procedures, examples, and intermediates are intended to exemplify embodiments of the disclosure. They are in no way intended to be limiting. Other embodiments of this disclosure may be prepared using the methods illustrated in these examples, either alone or in combination with techniques generally known in the art.

### Example 1: Preparation of (R)—N-(2-(4-Cyanothiazolidin-3-yl)-2-oxoethyl)-6-morpholinoquinoline-4-carboxamide

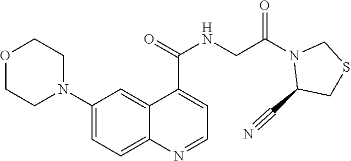 [SMILES: N#C[C@@H]1CSCN1C(=O)CNC(=O)c1ccnc2ccc(N3CCOCC3)cc12]

(R)—N-(2-(4-Cyanothiazolidin-3-yl)-2-oxoethyl)-6-morpholinoquinoline-4-carboxamide can be prepared as described below:

### A. (R)-3-Glycylthiazolidine-4-carbonitrile hydrochloride (Intermediate 1)

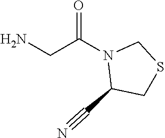 [SMILES: N#C[C@@H]1CSCN1C(=O)CN]

### (i) tert-Butyl (R)-4-carbamoylthiazolidine-3-carboxylate (Intermediate 1-A)

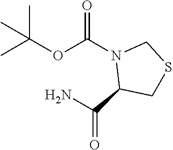

Di-tert-butyl dicarbonate (Boc₂O, 18.6 mL, 80.2 mmol) was added to a stirred solution of (R)-3-(tert-butoxycarbonyl) thiazolidine-4-carboxylic acid (17.0 g, 72.9 mmol) and pyridine (7.07 mL, 87.5 mmol) in ethyl acetate (170 mL) and the reaction mixture was stirred at room temperature for 3 hours. Then, a solution of NH₃ (aq. 25%, 6 mL) was added dropwise and the mixture was stirred at room temperature overnight. The reaction mixture was diluted with ethyl acetate, the phases were separated, and the organic phase was washed with sat. NaCl, dried, filtered through a pad of silica gel, washed with ethyl acetate, and evaporated to give the crude tert-butyl (R)-4-carbamoylthiazolidine-3-carboxylate (Intermediate 1-A, 16.9 g, 100%) as a colorless oil, which was used directly in the next step.

### (ii) tert-Butyl (R)-4-cyanothiazolidine-3-carboxylate (Intermediate 1-B)

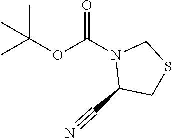

Trifluoro acetic acid anhydride (TFAA, 12.4 mL, 87.5 mmol) as a solution in ethyl acetate (20 mL) was added to a solution of crude tert-butyl (R)-4-carbamoyl-thiazolidine-3-carboxy late (Intermediate 1-A, 16.9 g, 72.9 mmol) and pyridine (14.7 mL, 182 mmol) in ethyl acetate (150 mL) at room temperature. The mixture was stirred at room temperature for 4 hours and then diluted with ethyl acetate, washed with aq. HCl (1 M), and sat. NaHCO₃. The organic phase was dried, filtered through a pad of silica gel, washed with ethyl acetate, and evaporated to give a light-yellow oil which solidified on standing. The crude solid material was suspended in heptane:ethyl acetate (4:1, 50 mL) and stirred at room temperature overnight. The solids were filtered off, washed with heptane: ethyl acetate (4:1), and dried to give tert-butyl (R)-4-cyanothiazolidine-3-carboxylate (Intermediate 1-B, 12.0 g, 83%) as a colorless solid; ¹H NMR (400 MHZ, CDCl₃) δ 5.20-4.79 (m, 1H), 4.60-4.53 (m, 1H), 4.53-4.36 (m, 1H), 3.40-3.18 (m, 2H), 1.51 (s, 9H).

### (iii) (R)-Thiazolidine-4-carbonitrile hydrochloride (Intermediate 1-C)

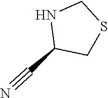

A solution of aq. HCl (12 M, 11 mL) in methanol (140 mL) was added slowly to a solution of tert-butyl (R)-4-cyanothiazolidine-3-carboxylate (Intermediate 1-B, 6.0 g, 28 mmol) in methanol (140 mL) at room temperature. The clear colorless solution was stirred at room temperature for 2 hours. Solvents were evaporated to give (R)-thiazolidine-4-carbonitrile hydrochloride (Intermediate 1-C, 4.22 g, 100%) as a colorless solid: ¹H NMR (400 MHZ, CD₃OD) δ 4.90 (dd, 1H), 4.35-4.24 (m, 2H), 3.37-3.24 (m, 2H).

### (iv) tert-Butyl (R)-(2-(4-cyanothiazolidin-3-yl)-2-oxoethyl)carbamate (Intermediate 1-D)

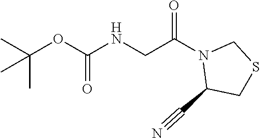 [SMILES: CC(C)(C)OC(=O)NCC(=O)N1CSC[C@H]1C#N]

N-Ethyl-N-isopropyl-propan-2-amine (DIPEA, 19.6 mL, 112 mmol) was added to a suspension of (R)-thiazolidine-4-carbonitrile hydrochloride (Intermediate 1-C, 4.22 g, 28 mmol), (tert-butoxycarbonyl) glycine (6.13 g, 35.0 mmol) and propanephosphonic acid anhydride (T₃P, 41.6 mL, 70.0 mmol, 50% solution in ethyl acetate) in ethyl acetate (120 mL). The mixture was heated at 60° C. for 4 hours. The mixture was diluted with ethyl acetate, and sequentially washed with water, aq. HCl (1 M) and sat. NaHCO₃. The organic phase was dried, filtered, and evaporated. The residue was filtered through a pad of silica gel, washed with heptane:ethyl acetate (1:1) and evaporated to give an oil which was triturated with heptane:DCM to give tert-butyl (R)-(2-(4-cyanothiazolidin-3-yl)-2-oxoethyl)carbamate (Intermediate 1-D, 7.60 g, 100%) as an almost colorless solid; ¹H NMR (400 MHZ, CDCl₃) δ 5.36-5.25 (m, 2H), 4.59-4.52 (m, 2H), 4.14-3.90 (m, 2H), 3.29 (d, 2H), 1.45 (s, 9H).

### (v) (R)-3-Glycylthiazolidine-4-carbonitrile hydrochloride (Intermediate 1)

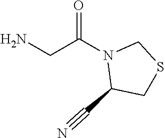 [SMILES: N#C[C@@H]1CSCN1C(=O)CN]

A solution of aq. HCl (12 M, 5.6 mL) in methanol (140 mL) was added slowly to a solution of tert-butyl (R)-(2-(4-cyanothiazolidin-3-yl)-2-oxoethyl) carbamate (Intermediate 1-D, 7.60 g, 28.0 mmol) in methanol (140 mL), then the solution was stirred at room temperature overnight. The solvents were evaporated to give (R)-3-glycylthiazolidine-4-carbonitrile hydrochloride (Intermediate 1, 5.80 g, 100%) as a colorless solid: ¹H NMR (400 MHZ, CD₃OD) δ 5.34 (t, 1H), 4.72 (d, 1H), 4.62 (d, 1H), 4.11-3.94 (m, 2H), 3.41-3.36 (m, 2H).

### B. 6-Morpholinoquinoline-4-carboxylic acid (Intermediate 2)

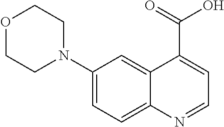 [SMILES: O=C(O)c1ccnc2ccc(N3CCOCC3)cc12]

### (i) Ethyl 6-morpholinoquinoline-4-carboxylate (Intermediate 2-A)

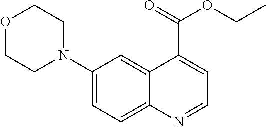 [SMILES: CCOC(=O)c1ccnc2ccc(N3CCOCC3)cc12]

Morpholine (0.22 mL, 2.5 mmol) was added to a mixture of ethyl 6-bromoquinoline-4-carboxylate (0.355 g, 1.27 mmol), bis(dibenzylideneacetone)palladium (Pd(dba)₂, 36 mg, 0.06 mmol), dicyclohexyl(2′,6′-diisopropoxy-[1,1′-biphenyl]-2-yl)phosphane (RuPhos, 59 mg, 0.13 mmol) and K₃PO₄ (0.538 g, 2.53 mmol) in 2-methylpropan-2-ol (2.3 mL). The flask was sealed, purged with N₂ (g), and heated at 90° C. overnight. The reaction mixture was diluted with ethyl acetate, washed sequentially with water and brine. The organic layer was dried by passing through a phase separator and concentrated under reduced pressure to give ethyl 6-morpholinoquinoline-4-carboxylate (Intermediate 2-A, 110 mg, 30%): MS m/z (ESI), [M+H]+ 287.2.

### (ii) 6-Morpholinoquinoline-4-carboxylic acid (Intermediate 2)

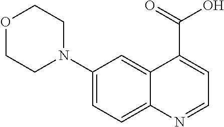 [SMILES: O=C(O)c1ccnc2ccc(N3CCOCC3)cc12]

NaOH (31 mg, 0.77 mmol) was added to a solution of ethyl 6-morpholinoquinoline-4-carboxylate (Intermediate 2-A, 110 mg, 0.38 mmol) in methanol (4 mL), and heated at 60° C. for 2 hours. The reaction mixture was cooled to room temperature, and aq HCl (0.023 mL) was added. The reaction mixture was concentrated under reduced pressure to give 6-morpholinoquinoline-4-carboxylic acid (Intermediate 2, 95 mg, 96%): MS m/z (ESI), [M+H]+ 259.1.

### C. (R)—N-(2-(4-Cyanothiazolidin-3-yl)-2-oxoethyl)-6-morpholinoquinoline-4-carboxamide

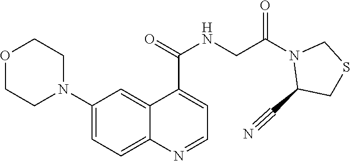 [SMILES: N#C[C@@H]1CSCN1C(=O)CNC(=O)c1ccnc2ccc(N3CCOCC3)cc12]

N-Ethyl-N-isopropyl-propan-2-amine (DIPEA, 0.15 mL, 0.87 mmol) was added to a suspension of 6-morpholinoquinoline-4-carboxylic acid (Intermediate 2, 75 mg, 0.29 mmol), (R)-3-glycylthiazolidine-4-carbonitrile hydrochloride (Intermediate 1, 121 mg, 0.58 mmol), 1-hydroxy benzotriazole hydrate (HoBt, 53 mg, 0.35 mmol), and 3-(ethyliminomethyleneamino)-N,N-dimethyl-propan-1-amine hydrochloride (EDC, 84 mg, 0.44 mmol) in ethyl acetate (1 mL) and acetonitrile (1 mL). Gives a clear yellow solution which was stirred at room temperature overnight. The mixture was diluted with ethyl acetate, washed with sat. NaHCO₃ and brine. The organic phase was dried, filtered, and evaporated. The residue was purified by preparative HPLC on a Kromasil C8 column (10 μm, 250×20 mm ID) using a gradient (5-65%) of acetonitrile in H₂O/acetonitrile/formic acid (95/5/0.2) as mobile phase to give (R)—N-(2-(4-cyanothiazolidin-3-yl)-2-oxoethyl)-6-morpholinoquinoline-4-carboxamide (26 mg, 21%): HRMS (ESI) m/z [M+H]+ calcd for C₂₀H₂₂N₅O₃S: 412.1438 found: 412.1437: ¹H NMR (400 MHZ, CD₃CN) δ 8.71 (d, 1H), 8.05 (s, 1H), 7.96 (d, 1H), 7.69 (d, 1H), 7.60 (dd, 1H), 7.43 (d, 1H), 5.24 (dd, 1H), 4.79-4.60 (m, 2H), 4.30 (d, 2H), 3.88-3.79 (m, 4H), 3.35-3.28 (m, 6H).

### Example 2: Analytical Methods

Unless otherwise stated, the following analytical methods were used to characterize the crystalline forms described in the following Examples:

### A. Reflection Powder X-Ray Diffraction (PXRD For Amorphous)

The X-ray diffraction analysis is performed according to standard methods, which can be found, for example, in Kitaigorodsky, A. I. (1973), Molecular Crystals and Molecules, Academic Press, New York: Bunn, C. W. (1948), Chemical Crystallography, Clarendon Press, London: or Klug, H. P. & Alexander, L. E. (1974), X-ray Diffraction Procedures, John Wiley & Sons, New York.

The powder X-ray diffraction (referred to herein as PXRD) pattern was determined by mounting a sample on a zero-background holder, single silicon crystal, and spreading out the sample into a thin layer. The PXRD is recorded with a Theta-Theta PANalytical X'Pert PRO (wavelength of X-rays 1.5418 Å nickel-filtered Cu radiation, Voltage 45 kV, filament emission 40 mA). Variable divergence and anti-scatter slits and incident and diffracted soller slit 0.04° are used. The samples are rotated during measurement. Samples are scanned from 2.4-50° 2θ using a 0.013° step width and a 115.770 s count time together with a PIXcel 1D detector (active length 3.35° 2θ). The PXRD patterns are obtained in Bragg-Brentano geometry.

One of skill in the art will recognize that a PXRD pattern may be obtained which has one or more measurement errors depending on measurement conditions, such as equipment or machine used (Jenkins, R & Snyder, R. L. ‘Introduction to X-Ray Powder Diffractometry’ John Wiley & Sons 1996; Bunn, C. W. (1948), Chemical Crystallography, Clarendon Press, London: Klug, H. P. & Alexander, L. E. (1974), X-Ray Diffraction Procedures). Those skilled in the art of X-ray powder diffraction will further recognize that the relative intensity of peaks can be affected by, for example, grains above 30 microns in size and non-unitary aspect ratios that may affect analysis of samples. Furthermore, it should be understood that intensities might fluctuate depending on experimental conditions and sample preparation (e.g., preferred orientation). The following definitions have been used for the relative intensity (%): 25%-100%, vs (very strong): 10%-25%, s (strong): 3%-10%, m (medium): 1%-3%, w (weak).

One of skill in the art will also recognize that the position of reflections can be affected by the precise height at which the sample sits in the diffractometer and the zero calibration of the diffractometer. The surface planarity of the sample may also have a small effect. Hence the diffraction pattern data presented are not to be taken as absolute values. Generally, a measurement error of a diffraction angle in an X-ray powder diffractogram may be approximately plus or minus 0.2 2θ, and such a degree of a measurement error should be taken into account when considering the PXRD data.

The reflection mode PXRD pattern may be compared to the transmission mode PXRD pattern, although those skilled in the art will realize that the diffraction patterns may vary, particularly with respect to peak intensities.

### B. Reflection Powder X-Ray-Diffraction (PXRD For Type 2)

The powder X-ray diffraction (referred to herein as PXRD) pattern was determined by mounting a sample on a zero-background holder, single silicon crystal, and spreading out the sample into a thin layer. The powder X-ray diffraction was recorded with a reflection Theta-Theta PANalytical Empyrean (wavelength of X-rays 1.5419 Å nickel-filtered Cu radiation, Voltage 45 kV, filament emission 40 mA. Variable divergence and anitscatter slits and incident and diffracted soller slit 0.04° were used. The samples were rotated during measurement. Samples were scanned from 2.4-50° 2Theta using a 0.013° step width and a 234.345 s counting time together with a PIXcel3D-Medipix3 detector (active length 3.35° 2θ). The following definitions have been used for the relative intensity (%): 25%-100%, vs (very strong): 10%-25%, s (strong): 3%-10%, m (medium); 1%-3%, w (weak).

### C. Transmission Powder X-Ray Diffraction (PXRD For Form A and Form B)

Powder X-ray diffraction data are measured with Corundum as an internal reference. The powder X-ray diffraction (PXRD) pattern is determined by mounting a sample between two Kapton® Polyimide films, forming a thin layer between the films. The PXRD is recorded with a transmission PANalytical Empyrean (wavelength of X-rays 1.5419 Å nickel-filtered Cu radiation, Voltage 45 kV, filament emission 40 mA). Fixed divergence and anti-scatter slits are used and the samples are rotated during measurement. Samples are scanned from 2.5-50° 2θ using a 0.013° step width and a 938.145 s counting time together with a PIXcel3D-Medipix3 detector (active length 3.35° θ). The following definitions have been used for the relative intensity (%): 25%-100%, vs (very strong): 10%-25%, s (strong): 3%-10%, m (medium): 1%-3%, w (weak).

### D. Solid-State ¹³C NMR Spectroscopy

Approximately 100 mg of material is packed into a 4 mm Zirconium Dioxide rotor sealed with a Kel-F cap. For determination of ¹³C Cross Polarization Magic Angle Spinning spectra the rotor is spun at 15 kHz (to remove chemical shift anisotropy) and the ¹³C spectrum is recorded using cross polarization from hydrogen (improves sensitivity and reduces experiment times). The contact time for the magnetization transfer is 2 milliseconds and the inter-pulse delay (allowing for nuclear relaxation) is 15 seconds. Signal averaging is employed with sufficient scans recorded to enable all the major peaks to be resolved from the noise (typically 1k scans). Glycine is used as a secondary chemical shift reference (main peak at 176.03 ppm).

### E. Ramp Differential Scanning Calorimetry (DSC)

The melting point temperature onset (Tₘ) is determined by Differential Scanning calorimetry using a TA Instruments DSC, model Q2000. A sample (approximately 1-3 mg) is weighed into an aluminum sample pan. The sample is packed to the bottom of the sample pan and a lid with a pin hole is used. The instrument is purged with nitrogen at 50 mL/min and data collected between 25° C. and 210-250° C., using a heating rate of 10° C./minute.

### F. MDSC Differential Scanning Calorimetry (DSC)

The glass transition temperature midpoint (Tg) is determined by MSC Differential Scanning calorimetry using a TA Instruments DSC, model Q2000. A sample (approximately 5 mg) is weighed into an aluminum sample pan. The sample is packed to the bottom of the sample pan and a lid with a pin hole is used. The instrument is purged with nitrogen at 50 mL/min and data collected between 25° C. and 230° C., using a heat only modulation method with an underlying heating rate of 5° C./minute and a modulating amplitude ±0.53° C. every 40 seconds.

### G. Thermogravimetric Analysis (TGA)

Thermal gravimetric analysis is performed using a TA Instruments TGA, model Q500. A sample (approximately 10 mg) is transferred to a tared sample holder. The instrument is purged with nitrogen, oven 60 mL/min and balance 40 mL/min, and data are collected between room temperature and 300° C., using a heating rate of 10° C./min. During heating, the buoyancy effect will result in an observed weight increase. This effect can be reduced by using more than 15 mg of material or performing a baseline subtraction on the sample curve.

### H. Gravimetric Vapor Sorption (GVS)

Gravimetric vapor sorption analysis is performed using a TA Instruments TGA, model Q5000SA. A sample (approximately 5-10 mg) is transferred to a tared sample holder. The instrument is purged with nitrogen, chamber 200 mL/min and balance 10 mL/min at 25° C. and data are collected at different relative humidity (% RH). Starting at 20% RH and going stepwise up to 80% RH, down stepwise to 0% RH, and eventually a second cycle going up to 90% RH and back to 0% RH. The equilibrium criteria for moving to next % RH is reached when the drift criteria (dm/dt) is below 0.002 for 10 min.

Hygroscopicity can be assessed, for example, according to the European Pharmacopoeia (EP) classification: non-hygroscopic: <0.2%: slightly hygroscopic: ≥0.2% and <2%: hygroscopic: ≥2% and <15%: very hygroscopic: ≥15%: deliquescent: sufficient water is absorbed to form a liquid: all values measured as weight increase at 80% RH and 25° C.).

### Example 3: Form A/Form B Mixtures

Initial crystallization efforts to isolate pure Form A and pure Form B often resulted in mixtures of Form A and Form B. Crystallization parameters such as temperature, time, and solvent composition impacted the type and quality of the final crystalline product obtained.

### A. 50/50 Mixture

In this study a mixture of Form A and Form B (˜50/50) was obtained when (R)—N-(2-(4-cyanothiazolidin-3-yl)-2-oxoethyl)-6-morpholinoquinoline-4-carboxamide was isolated from a mixture of isopropanol and ethyl acetate. Specifically, 6-morpholino-quinoline-4-carboxylic acid (Intermediate 2 of Example 1, 177 g, 0.757 mol, 1.0 eq.) was charged into a 5 L jacket vessel. (R)-3-glycylthiazolidine-4-carboxamide (159 g, 1.2 eq.) and then ethyl acetate (3500 mL) were charged to the reactor. A 50% wt/wt solution of propylphosphonic anhydride in ethyl acetate (1310 g, 3 eq.) was then charged to the reactor followed by DIPEA (443 g. 5.002 eq.). The temperature was adjusted to 65° C. and the reaction mixture stirred for 48 hours followed by cooling to 20° C.

The reaction mixture was combined with a second reaction mixture prepared in a similar manner (total 465 g) and charged into a 50 L vessel for workup. Ethyl acetate (20 L) was charged to the vessel and the pH was adjusted to around 9 using 10 L sat. aq. NaHCO₃. The resulting biphasic mixture was stirred for 15 minutes at 20° C. followed by separation of layers. The aqueous phase was extracted by 10 L ethyl acetate and stirred for 15 minutes at 20° C. followed by separation of phases. The organic phase was washed by 10 L sat. aq. NaHCO₃ solution and stirred for 15 minutes at 20° C. followed by separation of layers. All the organic phases were combined and concentrated to about 5 volumes using a rotary evaporator at 45° C. The organic phase was passed through a 5 kg silica Pad using isopropanol (15 L) as the eluent. The organic phase was then concentrated to about 5 volumes using a rotary evaporator at 45° C., the concentrated organic phase swapped with 10 L ethyl acetate and concentrated to about 8 volumes. The suspension was filtered and the wet cake washed with 2 L ethyl acetate. The wet cake was dried at 50° C. for 18 hours yielding a light yellow solid that was approximately a 50/50 mixture of Form A and Form B of (R)—N-(2-(4-cyanothiazolidin-3-yl)-2-oxoethyl)-6-morpholinoquinoline-4-carboxamide.

### B. Additional Studies

A small-scale crystallization study was conducted. Amorphous (R)—N-(2-(4-cyanothiazolidin-3-yl)-2-oxoethyl)-6-morpholino-quinoline-4-carboxamide was suspended or dissolved in each of the solvents listed in Table 1 below (20 relative volumes) and stirred for 3 days at 20° C. Each solid obtained was then analyzed by PXRD and DSC. Results are reported in Table 1 below.

TABLE 1

| SOLVENT | RESULT |
|---|---|
| Ethyl Acetate | Form A/Form B Mixture |
| | (About 85/15 based on DSC) |
| Acetone | Form B |
| Methyl isobutyl ketone | Form A |
| Methyl Ethyl Ketone | Form B |
| Acetonitrile | Form B |

Use of ethyl acetate or methyl isobutyl ketone as crystallization solvents appeared to favor formation of Form A while use of acetone, methyl ethyl ketone, or acetonitrile appeared to favor formation of Form B. Other crystallization parameters selected, however, also appear to influence the crystalline form produced. In Example 3A above, a solvent system comprising isopropanol and ethyl acetate produced a 50/50 mixture of Form A and Form B. The low temperature used during concentration procedure and the limited time, however, might not have been sufficient to allow Form A to dissolve and transform to form B. In Example 5A (v) below, pure Form B was obtained starting from a 50/50 mixture of Form A and Form B using a solvent system comprising acetone and ethyl acetate. The suspension was stirred at a high temperature for several hours which may have allowed most of the lower melting point Form A to dissolve which then enriched the supension with the higher melting Form B over time allowing the Form B present to act as seed crystals as the suspension was cooled to 20° C.

### Example 4: Crystalline Form A

### A. Preparation of Crystalline Form A

Amorphous (R)—N-(2-(4-cyanothiazolidin-3-yl)-2-oxoethyl)-6-morpholino-quinoline-4-carboxamide (19 g) was suspended in ethyl acetate (˜450 mL) and the resulting fine suspension was heated to 75° C. A fine yellow suspension was obtained, slowly cooled to 20° C. over 5 hours, and then stirred for an additional 12 hours at 20° C. The fine suspension was filtered and the yellow solid was washed with ethyl acetate (2×50 mL). The solid collected was first sucked relatively dry under vacuum at 20° C. for 10 minutes and then at 40° C. for 2 hours and finally dried under vacuum over the weekend at 35° C. PXRD and DSC analysis showed mainly pure Form A.

### B. Physical Characterization of Crystalline Form A

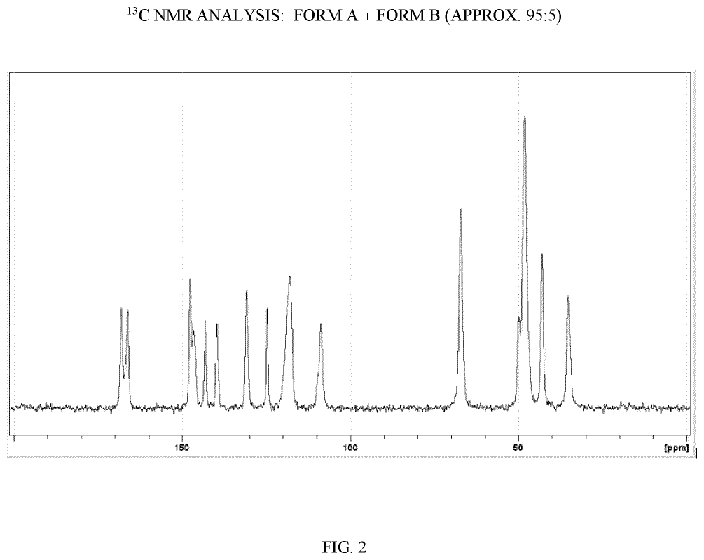

Characterization of Form A was carried out using various techniques including PXRD (FIG. 1), solid-state ¹³C NMR spectroscopy (FIGS. 2 to 5), differential scanning calorimetry (DSC) (FIG. 6), thermogravimetic analysis (TGA) (FIG. 7), and gravimetric vapor sorption (GVS) (FIG. 8).

The PXRD pattern of FIG. 1 confirms that Form A is crystalline. FIG. 1 shows the PXRD pattern for Form A measured using transmission geometry. Table 2 below lists selected peaks identified in the PXRD pattern of FIG. 1.

TABLE 2

| PEAK | RELATIVE | |
|---|---|---|
| POSITION | INTENSITY | |
| (°2θ) | (%) | DEFINITION |
| 8.9 | 15 | s |
| 12.0 | 72 | vs |
| 13.1 | 12 | s |
| 14.4 | 10 | m |
| 17.5 | 22 | s |
| 17.9 | 17 | s |
| 18.4 | 36 | vs |
| 19.8 | 24 | s |
| 20.6 | 17 | s |
| 21.1 | 23 | s |
| 21.7 | 100 | vs |
| 23.6 | 10 | m |
| 25.6 | 6 | m |
| 27.3 | 6 | m |
| 31.2 | 7 | m |
| 39.9 | 6 | m |
| 42.0 | 6 | m |

Form A shows distinctive peaks (relative to other forms except Form B) at 12.0±0.2° 2θ, 18.4±0.2° 2θ, 19.8±0.2° 2θ, and 21.7° 2θ±0.2° 2θ. Form A shows further characteristic peaks at 13.1±0.2° 2θ, 14.4±0.2° 2θ, 17.5±0.2° 2θ, and 21.1°20±0.2° 2θ. Form A lacks distinctive peaks (i.e., medium or stronger relative intensity peaks) relative to Form B at 18.7±0.2° 2θ and 22.4° 2θ±0.2° 2θ.

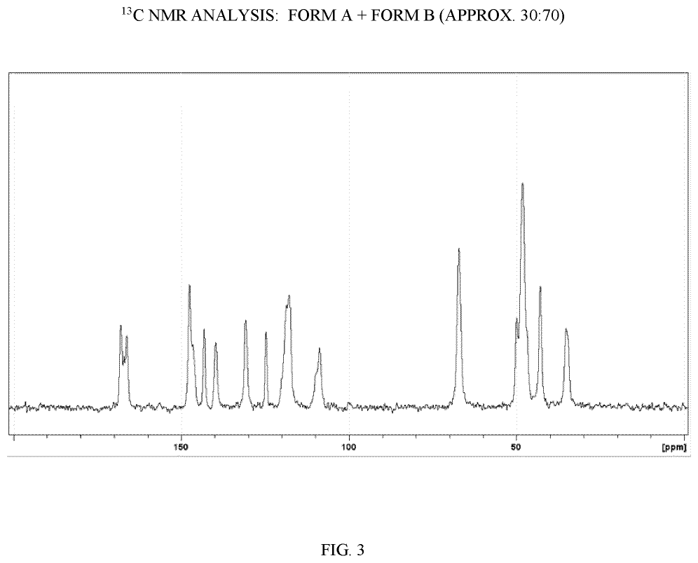

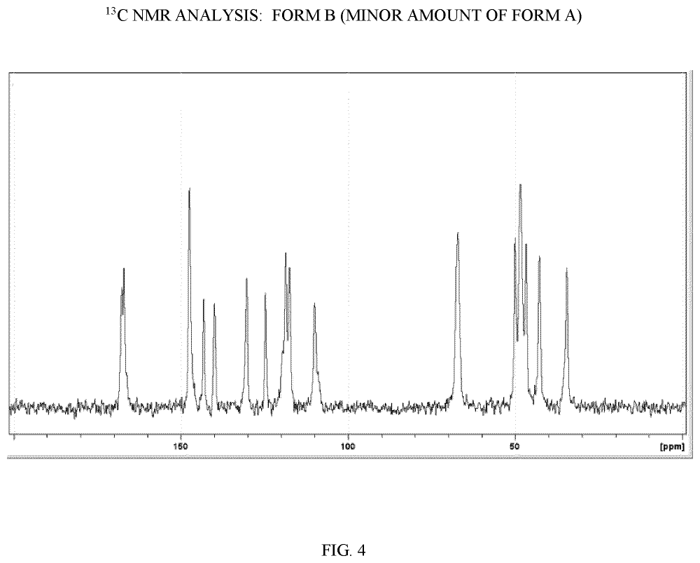

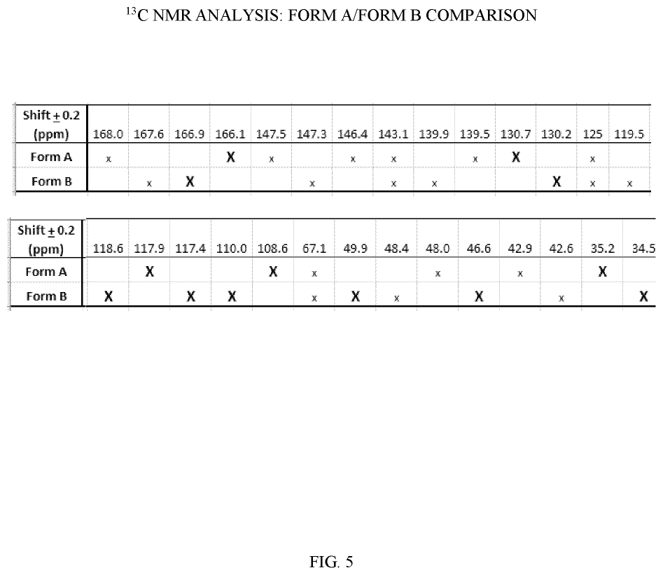

FIG. 2 shows a representative solid-state ¹³C NMR spectrum for a sample of crystalline (R)—N-(2-(4-cyanothiazolidin-3-yl)-2-oxoethyl)-6-morpholinoquinoline-4-carboxamide that is approximately 95 weight % Form A and 5 weight % Form B. FIG. 3 shows a representative solid-state ¹³C NMR spectrum for a sample of crystalline (R)—N-(2-(4-cyanothiazolidin-3-yl)-2-oxoethyl)-6-morpholinoquinoline-4-carboxamide that is approximately 30 weight % Form A and 70 weight % Form B. FIG. 4 shows a representative solid-state ¹³C NMR spectrum for a sample of crystalline (R)—N-(2-(4-cyanothiazolidin-3-yl)-2-oxoethyl)-6-morpholinoquinoline-4-carboxamide that is Form B with a minor amount of Form A. FIG. 5 is a comparison of the solid-state ¹³C NMR spectra for crystalline (R)—N-(2-(4-cyanothiazolidin-3-yl)-2-oxoethyl)-6-morpholinoquinoline-4-carboxamide Form A and Form B based on an analysis of the combined spectra of FIGS. 2, 3, and 4. Form A shows characteristic peaks at 168.0±0.2 ppm, 166.1±0.2 ppm, 147.5±0.2 ppm, 146.4±0.2 ppm, 143.1±0.2 ppm, 139.5±0.2 ppm, 130.7±0.2 ppm, 125±0.2 ppm, 117.9±0.2 ppm, 108.6±0.2 ppm, 67.1±0.2 ppm, 48.0±0.2 ppm, 42.9±0.2 ppm, and 35.2±0.2 ppm. Form A shows distinctive peaks relative to Form B at 166.1±0.2 ppm, 130.7±0.2 ppm, 117.9±0.2 ppm, 108.6±0.2 ppm, and 35.2±0.2 ppm.

FIG. 6 shows a representative Ramp DSC thermogram for Form A. Exothermic events are plotted in the upward direction. The melting endotherm shown in FIG. 6 has an onset temperature of about 171° C. and a heat enthalpy of approximately 68 J/g for the melting endotherm. The DSC values obtained can vary by as much as ±5° C. depending upon the instrument used, how samples are prepared, and differences between batches.

FIG. 7 shows a representative TGA thermogram for Form A. Form A exhibited a weight loss of less than about 0.1% upon heating from about 25° C. to 110° C., which confirms that Form A is an anhydrate.

FIG. 8 shows a representative GVS plot for Form A. Form A exhibited a reversible moisture uptake of about 0.05 weight % between 0% relative humidity and 80% relative humidity at 25° C.±0.1° C. The desorption curve indicates that Form A lost moisture at a similar rate to the moisture gained during sorption, with limited hysteresis. No form change was observed by PXRD after the GVS experiment. According to the European Pharmacopoeia (EP) classification, Form A is non-hygroscopic (i.e., <0.2% weight increase).

### Example 5: Crystalline Form B

### A. Preparation of Crystalline Form B

### (i) Unseeded Crystallization (Acetone)

Amorphous (R)—N-(2-(4-cyanothiazolidin-3-yl)-2-oxoethyl)-6-morpholino-quinoline-4-carboxamide (30 mg) was dissolved in 0.4 mL of acetone. After 30 minutes when the solution gradually became thicker, more acetone (0.2 mL) was added which resulted in a fine suspension. The suspension was allowed to stir for three days. The fine suspension was filtered and the solid was washed with acetone (0.6 mL). The solid was identified as Form B by DSC analysis.

### (ii) Unseeded Crystallization (Methyl Ethyl Ketone)

Amorphous (R)—N-(2-(4-cyanothiazolidin-3-yl)-2-oxoethyl)-6-morpholino-quinoline-4-carboxamide (30 mg) was dissolved in 0.4 mL of methyl ethyl ketone. After 15 minutes, when the solution gradually became thicker, more methyl ethyl ketone (0.2 mL) was added which resulted in a fine suspension. The suspension was allowed to stir for three days. The fine suspension was filtered and the solid was washed with methyl ethyl ketone (0.6 mL). The solid was identified as Form B by DSC analysis.

(iii) Unseeded Crystallization (Acetonitrile)

Amorphous (R)—N-(2-(4-cyanothiazolidin-3-yl)-2-oxoethyl)-6-morpholino-quinoline-4-carboxamide (30 mg) was dissolved in 0.4 mL of acetonitrile. After 45 minutes, the solution gradually became thicker, and after 2 hours more acetonitrile (0.2 mL) was added which resulted in a fine suspension. The suspension was allowed to stir for three days. The fine suspension was filtered and the solid was washed with acetonitrile (0.6 mL). The solid was identified as Form B by DSC analysis.

### (iv) Seeded Crystallization (Acetonitrile)

(R)—N-(2-(4-cyanothiazolidin-3-yl)-2-oxoethyl)-6-morpholinoquinoline-4-carboxamide (“Quinoline”, 6.0 g) was dissolved in 90 mL acetonitrile at 70° C. and the resulting solution was filtered through a glass microfibre filter. The filter was washed with 3.0 mL acetonitrile and the combined filtrates were cooled to 40° C. at a rate of no more than 1.5° C./minute. The solution was seeded with crystalline (R)—N-(2-(4-cyanothiazolidin-3-yl)-2-oxoethyl)-6-morpholinoquinoline-4-carboxamide Form B (150 mg/0.025 equivalents, pre-ground in a pestle and mortar) and then agitated at 40° C. for at least 14 hours. The resulting slurry was cooled to 15° C. at a rate of 5° C./hour before isolating the product by filtration under vacuum. The filter cake was washed sequentially with 6 mL acetonitrile and 12 mL tert-butyl methyl ether before drying under vacuum at 40° C. to yield the crystalline Form B final product (4.55 g).

The above-described procedure also worked well when the solution was instead seeded with crystalline (R)—N-(2-(4-cyanothiazolidin-3-yl)-2-oxoethyl)-6-morpholino-quinoline-4-carboxamide Form B (90 mg/0.015 equivalents, pre-ground in a pestle and mortar).

As noted above, seeding with 0.015 to 0.025 equivalents of ground Form B seed worked well for acetonitrile solutions. Further modifications of the seeding procedure were evaluated as described in Table 3 below:

TABLE 3

| SEEDING CONDITIONS¹ | RESULT |
|---|---|
| Solution seeded with 0.03 equivalents of | Did not induce significant |
| unground Form B seed and held at 40° C. | crystallization at 40° C. or |
| | after cooling to and holding |
| | at 20° C. for 22 hours. |
| Solution seeded with 0.001 equivalents of | Significant crystallization |
| pre-ground Form B seed and held at 40° C. | was not observed during a |
| | hold of about 24 hours. In |
| | one run, crystallization |
| | started during a subsequent |
| | cool to 30° C. |
| Quinoline (6.5 g) dissolved in 100 mL of | Significant crystallization |
| acetonitrile at 25° C. and solution seeded | was not observed until |
| with 0.001 equivalents of pre-ground | 2 days after seeding. |
| Form B seed | |
| Quinoline (25.5 g) dissolved in 395 mL of | Significant crystallization |
| acetonitrile at 25° C. and solution seeded | was not observed until |
| with 0.001 equivalents of pre-ground | 8 hours after seeding. |
| Form B seed | |
| Quinoline (6.0 g) dissolved in 93 mL of | Significant crystallization |
| acetonitrile at 20° C. and solution seeded | was not observed until |
| with 0.001 equivalents of pre-ground | 2 hours of seeding. |
| Form B seed | |
| Quinoline (6.5 g) dissolved in 100 mL of | Significant crystallization |
| acetonitrile at 35° C. and solution seeded | was not observed until |
| with 0.001 equivalents of pre-ground | 14 hours after seeding. |
| Form B seed | |

¹The seed was ground in portions so may not have been consistent across these experiments.

### (v) Form A/Form B Interconversion

Crystalline (R)—N-(2-(4-cyanothiazolidin-3-yl)-2-oxoethyl)-6-morpholino-quinoline-4-carboxamide (50/50 mixture of Form A and Form B, 20 g, 48.61 mmol) was charged to a reactor equipped with an overhead stirrer followed by addition of acetone (225 mL) and then ethyl acetate (75 mL). The resulting yellow suspension was slowly heated to reflux (T_{jacket}=70° C.) and stirred (300 rpm) at that temperature for 5 hours followed by cooling to 20° C. over 5 hours. The suspension was then stirred for an additional 15 hours at 20° C. A very fine, easily stirred suspension was obtained with no solid attached to the glass walls of the reactor. The mixture was filtered and the solid washed with 25% ethyl acetate in acetone (80 mL). The filter cake was then sucked dry for 5 minutes. Further drying over night under vacuum at 40° C. gave 15 g of a yellow product. PXRD and DSC analysis (melting point of 194° C.) were consistent with Form B.

### (vi) Seeded Crystallization (Methanol)

(R)—N-(2-(4-cyanothiazolidin-3-yl)-2-oxoethyl)-6-morpholino-quinoline-4-carboxamide (4.5 g) was dissolved in 100 mL of methanol at 60° C. The resulting solution was cooled to 45° C. at a rate of 5.0° C./minute. The solution was then seeded with crystalline (R)—N-(2-(4-cyanothiazolidin-3-yl)-2-oxoethyl)-6-morpholino-quinoline-4-carboxamide Form B (0.0008 equivalents, pre-ground in a pestle and mortar) and agitated at 45° C. for 2 days. The solution was then cooled to 20° C. and held at 20° C. The most substantial crystallization was observed around 2 days after cooling to 20° C. PXRD and DSC analysis were consistent with Form B, but additional studies (see Example 7) suggest that (R)—N-(2-(4-cyanothiazolidin-3-yl)-2-oxoethyl)-6-morpholino-quinoline-4-carboxamide may be chemically unstable in methanol.

(vii) Seeded Crystallization (Acetonitrile/1-Butanol)

As holding time increases for (R)—N-(2-(4-cyanothiazolidin-3-yl)-2-oxoethyl)-6-morpholino-quinoline-4-carboxamide in an acetonitrile solution at 70° C., the risk of racemisation also increases. To further reduce this risk, (R)—N-(2-(4-cyanothiazolidin-3-yl)-2-oxoethyl)-6-morpholino-quinoline-4-carboxamide (12.6 g) was dissolved in a mixture of acetonitrile (31.5 mL) and 1-butanol (58.3 mL) at 70° C. and the resulting solution was filtered through a glass microfibre filter. The filter was washed with a mixture of acetonitrile (1.1 mL) and 1-butanol (2.0 mL) and the combined filtrates were cooled to 44° C. at a rate of 1.73° C./minute. The solution was seeded with with crystalline (R)—N-(2-(4-cyanothiazolidin-3-yl)-2-oxoethyl)-6-morpholino-quinoline-4-carboxamide Form B (4.3 mg, 0.00035 equivalents, pre-ground in a pestle and mortar) and then agitated at 44° C. for at least 12 hours. The resulting slurry was cooled to 10° C. at a rate of 4.9° C./hour before isolating the product by filtration under vacuum. The filter cake was washed sequentially with 1-butanol (12.4 mL) and tert-butyl methyl ether (24.8 mL) before drying under vacuum at 40° C. PXRD and DSC analysis were consistent with Form B.

### B. Physical Characterization of Crystalline Form B

Characterization of Form B was carried out using various techniques including PXRD (FIGS. 9 to 11), solid-state ¹³C NMR spectroscopy (FIGS. 2 to 5), differential scanning calorimetry (DSC) (FIG. 12), thermogravimetic analysis (TGA) (FIG. 13), and gravimetric vapor sorption (GVS) (FIG. 14).

The PXRD pattern of FIG. 9 confirms that Form B is crystalline. FIG. 9 shows the PXRD pattern for Form B measured using transmission mode. Table 4 below lists selected peaks identified in the PXRD pattern of FIG. 9.

TABLE 4

| PEAK | RELATIVE | |
|---|---|---|
| POSITION | INTENSITY | |
| (°2θ) | (%) | DEFINITION |
| 8.9 | 6 | m |
| 12.0 | 39 | vs |
| 13.1 | 7 | m |
| 14.4 | 6 | m |
| 17.5 | 14 | s |
| 18.0 | 7 | m |
| 18.4 | 22 | s |
| 18.7 | 19 | s |
| 19.8 | 34 | vs |
| 20.3 | 10 | m |
| 20.6 | 13 | s |
| 21.1 | 9.6 | m |
| 21.7 | 100 | vs |
| 22.4 | 30 | vs |
| 22.9 | 8 | m |
| 25.2 | 5 | m |
| 25.6 | 6 | m |
| 26.2 | 5 | m |
| 28.7 | 5 | m |
| 30.4 | 5 | m |
| 30.9 | 5 | m |
| 31.1 | 5 | m |

Form B shows distinctive peaks (relative to other forms except Form A) at 12.0±0.2° 2θ, 18.4±0.2° 2θ, 18.7±0.2° 2θ, 19.8±0.2° 2θ, 21.7±0.2° 2θ, and 22.4° 2θ±0.2° 2θ. Form B shows further characteristic peaks at 13.1±0.2° 2θ, 14.4±0.2° 2θ, 17.5±0.2° 2θ, 20.3±0.2° 2θ, and 21.1°2θ±0.2° 2θ.

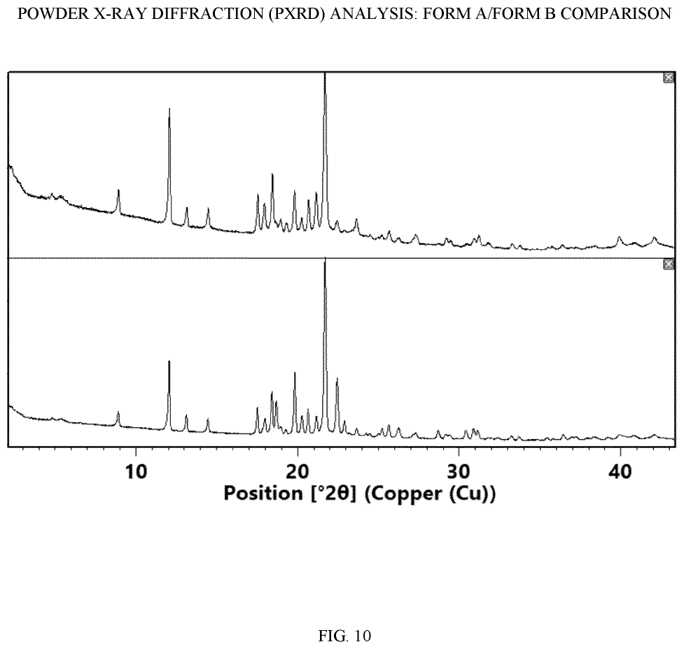

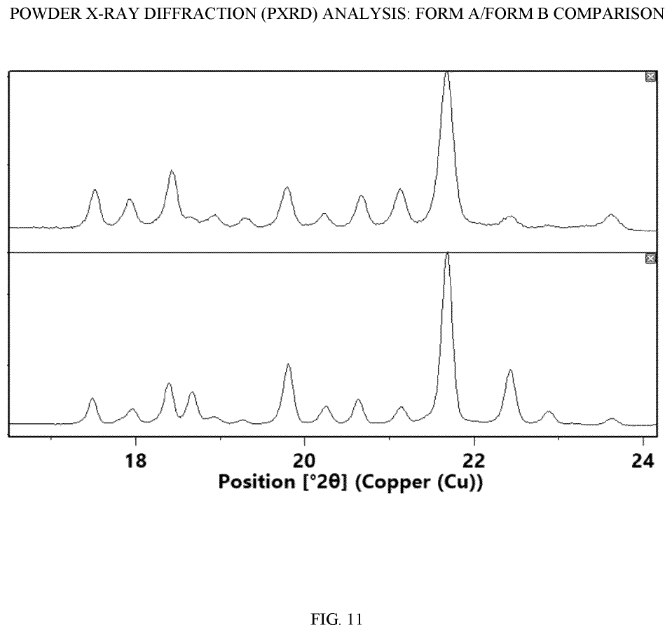

Form B shows distinctive peaks (i.e., medium or stronger relative intensity peaks) relative to Form A at 18.7±0.2° 2θ and 22.4° 2θ±0.2° 2θ. FIG. 10 is a comparison of the transmission powder X-ray diffraction patterns for Form A and Form B based on the diffractograms shown in FIGS. 1 and 9. FIG. 11 is a comparison of the Form A and Form B transmission powder X-ray diffraction patterns for region encompassing the distinctive Form B peaks. The Form A diffractogram is at the top and the Form B diffractogram is at the bottom in FIGS. 10 and 11.

As previously discussed for Form A, FIG. 2 shows a representative solid-state ¹³C NMR spectrum for a sample of crystalline (R)—N-(2-(4-cyanothiazolidin-3-yl)-2-oxoethyl)-6-morpholinoquinoline-4-carboxamide that is approximately 95 weight % Form A and 5 weight % Form B. FIG. 3 shows a representative solid-state ¹³C NMR spectrum for a sample of crystalline (R)—N-(2-(4-cyanothiazolidin-3-yl)-2-oxoethyl)-6-morpholino-quinoline-4-carboxamide that is approximately 30 weight % Form A and 70 weight % Form B. FIG. 4 shows a representative solid-state ¹³C NMR spectrum for a sample of crystalline (R)—N-(2-(4-cyanothiazolidin-3-yl)-2-oxoethyl)-6-morpholino-quinoline-4-carboxamide that is Form B with a minor amount of Form A. FIG. 5 is an analysis of the solid-state ¹³C NMR spectra for crystalline (R)—N-(2-(4-cyanothiazolidin-3-yl)-2-oxoethyl)-6-morpholino-quinoline-4-carboxamide Form A and Form B based on the combined spectra of FIGS. 2, 3, and 4. Form B shows characteristic peaks at 167.7±0.2 ppm, 166.9±0.2 ppm, 147.3±0.2 ppm, 143.1±0.2 ppm, 139.9±0.2 ppm, 130.2±0.2 ppm, 125±0.2 ppm, 119.5±0.2 ppm, 118.6±0.2 ppm, 117.4±0.2 ppm, 110.0±0.2 ppm, 49.9±0.2 ppm, 46.6±0.2 ppm, and 34.5±0.2 ppm. Form B shows distinctive peaks relative to Form A at 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

A representative Ramp DSC thermogram for Form B is shown in FIG. 12. Exothermic events are plotted in the upward direction. The melting endotherm shown in FIG. 12 has an onset temperature of about 191° C. and a heat enthalpy of approximately 87 J/g for the melting endotherm. As previously noted, DSC values obtained can vary by as much as ±5° C. depending upon the instrument used, how samples are prepared, and differences between batches. The DSC thermogram shown in FIG. 10 was generated using a DSC Q2000 module. The instrument was equilibrated at 25° C. and the sample (1 mg to 3 mg) heated to 230° C. at a rate of 10° C./minute.

FIG. 13 shows a representative TGA thermogram for Form B. Form B exhibited a weight loss less than about 0.1% upon heating from about 25° C. to 110° C. which confirms that Form B is an anhydrate.

FIG. 14 shows a representative GVS plot for Form B. Form B exhibited a reversible moisture uptake less than about 0.03 weight % between 0% relative humidity and 80% relative humidity at 25° C.±0.1° C. The desorption curve indicates that Form B lost moisture at a similar rate to the moisture gained during sorption, with limited hysteresis. No form change was observed by PXRD after the GVS experiment. According to the European Pharmacopoeia (EP) classification, Form B is non-hygroscopic (i.e., <0.2% weight increase).

### Example 6: Crystalline Type 2

Type 2 is a crystalline form of racemic N-(2-(4-cyanothiazolidin-3-yl)-2-oxoethyl)-6-morpholinoquinoline-4-carboxamide.

A. Preparation of (R,S)—N-(2-(4-Cyanothiazolidin-3-yl)-2-oxoethyl)-6-morpholinoquinoline-4-carboxamide Crystalline Type 2

(R,S)—N-(2-(4-cyanothiazolidin-3-yl)-2-oxoethyl)-6-morpholinoquinoline-4-carboxamide Type 2 can be isolated by slurrying (R)—N-(2-(4-cyanothiazolidin-3-yl)-2-oxoethyl)-6-morpholinoquinoline-4-carboxamide at high temperature in methanol or ethanol water mixtures for a long time or by several temperature cycles in chloroform, dioxane, or tetrahydrofuran. These methods are illustrated below.

### (i) Slurry at 50° C.

(R)—N-(2-(4-cyanothiazolidin-3-yl)-2-oxoethyl)-6-morpholinoquinoline-4-carboxamide (30-40 mg) Form B was slurried in 0.25 mL of a water mixture with methanol, with a volumetric ratio of 84:16 (methanol:water), at 50° C. for 7 days or more. The remaining solid material was isolated and allowed to dry before analysis. Analysis confirmed that the crystalline form isolated was Type 2.

### (ii) Temperature Cycling

About 50 mg of (R)—N-(2-(4-cyanothiazolidin-3-yl)-2-oxoethyl)-6-morpholino-quinoline-4-carboxamide amorphous was slurried in 0.3 mL of solvent (chloroform, dioxane, or tetrahydrofuran). The slurries were exposed to 10 temperature cycles between 5° C. and up to 3° C. below the boiling point of respectively solvent. The temperature cycling used a ramp of 1° C./min, when going up and down. The suspension was centrifuged, the supernatant was decanted, and the solid material allowed to dry before analysis. Analysis confirmed that the crystalline form isolated was Type 2.

### B. Physical Characterization of Crystalline Type 2

Characterization of Type 2 was carried out using various techniques including PXRD (FIG. 15), differential scanning calorimetry (DSC) (FIG. 16), and gravimetric vapor sorption (GVS) (FIG. 17).

The PXRD pattern of FIG. 15 confirms that Type 2 is crystalline. FIG. 15 shows the PXRD pattern for Type 2 measured using reflection geometry. Table 5 below lists selected peaks identified in the PXRD pattern of FIG. 15.

TABLE 5

| PEAK | RELATIVE | |
|---|---|---|
| POSITION | INTENSITY | |
| (°2θ) | (%) | DEFINITION |
| 8.1 | 4 | m |
| 10.0 | 100 | vs |
| 12.9 | 49 | vs |
| 15.6 | 19 | s |
| 15.8 | 34 | vs |
| 16.2 | 46 | vs |
| 17.1 | 52 | vs |
| 17.6 | 13 | s |
| 17.9 | 22 | s |
| 19.2 | 9 | m |
| 19.3 | 10 | m |
| 20.7 | 21 | s |
| 21.4 | 65 | vs |
| 22.0 | 75 | vs |
| 22.8 | 75 | vs |
| 23.5 | 20 | s |
| 24.0 | 39 | vs |
| 24.5 | 14 | s |
| 26.1 | 61 | vs |
| 26.5 | 57 | vs |
| 26.9 | 35 | vs |
| 29.5 | 9 | m |
| 30.3 | 12 | s |
| 30.7 | 11 | s |
| 31.1 | 8 | m |
| 31.7 | 9 | m |
| 32.7 | 5 | m |
| 34.0 | 7 | m |
| 37.7 | 13 | m |

Type 2 shows distinctive peaks (relative to other crystalline anhydrates) at 10.0±0.2° 2θ, 12.9±0.2° 2θ, 17.1±0.2° 2θ, 22.0±0.2° 2θ, and 22.8° 2θ±0.2° 2θ. Type 2 shows further characteristic peaks at 15.8±0.2° 2θ, 16.2±0.2° 2θ, 26.0±0.2° 2θ, 26.5±0.2° 2θ, and 26.9° 2θ=0.2° 2θ.

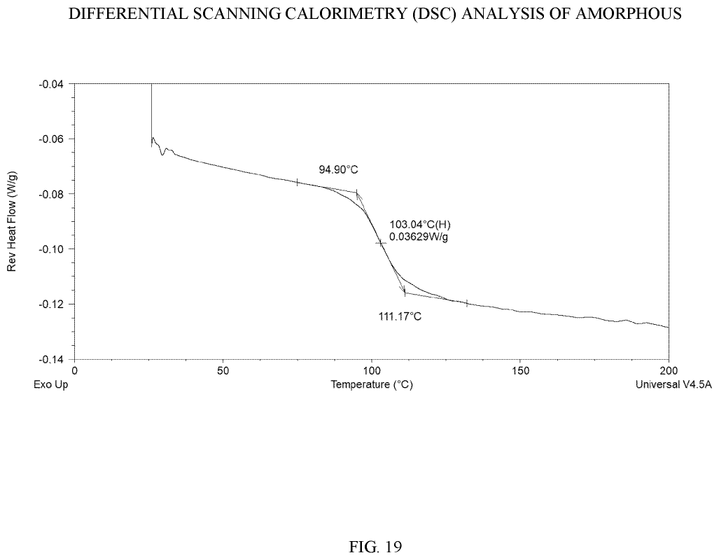

FIG. 16 shows a representative Ramp DSC thermogram for Type 2. Exothermic events are plotted in the upward direction. The melting endotherm shown in FIG. 19 has an onset temperature of about 201° C. with a heat flow of approximately 92 J/g for the melting endotherm. As previously noted, the DSC values obtained can vary by as much as ±5° C. depending upon the instrument used, how samples are prepared, and differences between batches.

FIG. 17 shows a representative GVS plot for Type 2. Type 2 exhibited a reversible moisture uptake less than about 0.5 weight % between 0% relative humidity and 80% relative humidity at 25° C.±0.1° C. The desorption curve indicates that Type 2 lost moisture at a similar rate to the moisture gained during sorption, with limited hysteresis. According to the European Pharmacopoeia (EP) classification, Type 2 is slightly hygroscopic (i.e., ≥0.2% and <2% weight increase).

### Example 7: Crystallization Solvents (Degradation/Epimerization)

Solutions of (R)—N-(2-(4-cyanothiazolidin-3-yl)-2-oxoethyl)-6-morpholino-quinoline-4-carboxamide (“Quinoline”, 1.5 mg/mL) in methanol, ethanol, and acetonitrile were held at ambient temperature and analyzed by supercritical fluid chromatography-mass spectrometry over a period of up to 14 days. New small peaks with masses corresponding to Quinoline+32 and Quinoline+46 were observed in methanol and ethanol respectively. It is believed that these peaks likely are from compounds generated by addition of methanol or ethanol to the nitrile of Quinoline, i.e., Compound 1 and Compound 2 and their respective enantiomers. The structures of Compound 1 and Compound 2 are shown below:

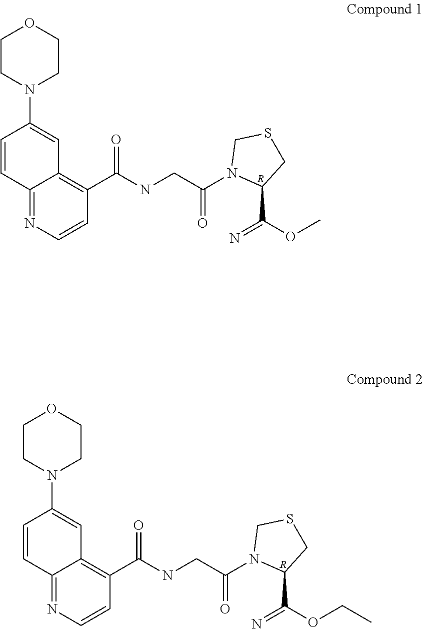

Small new peaks with mases corresponding to the proposed degradants, Compound 3 and Compound 4, also were observed in methanol and in ethanol. In addition, some epimerisation of (R)—N-(2-(4-cyanothiazolidin-3-yl)-2-oxoethyl)-6-morpholino-quinoline-4-carboxamide was observed. The structures of Compound 3 and Compound 4 are shown below:

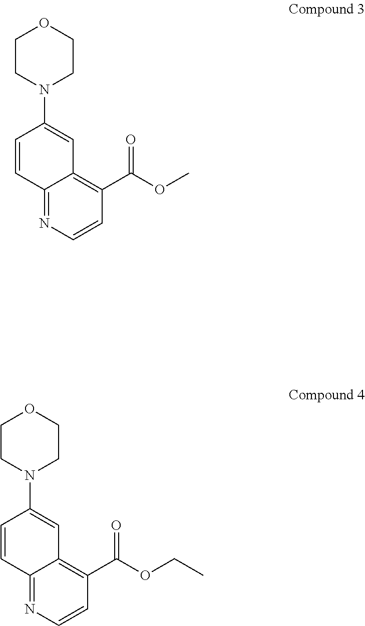 [SMILES: COC(=O)c1ccnc2ccc(N3CCOCC3)cc12 ; CCOC(=O)c1ccnc2ccc(N3CCOCC3)cc12]

Table 6 below reports the area % data over time measured for solutions held in methanol.

TABLE 6

| | t0 | 24 h | 48 h | 4 d | 7 d | 14 d |
|---|---|---|---|---|---|---|
| RT 1.5 min (Compound 3) | — | — | — | — | 0.02 | 0.02 |
| RT 3.2 min (Compound 1 | — | — | — | 0.02 | 0.03 | 0.08 |
| Enantiomer) | | | | | | |
| RT 3.4 min (Compound 1) | — | 0.05 | 0.13 | 0.22 | 0.36 | 0.54 |
| Quinoline Enantiomer | 1.18 | 1.18 | 1.20 | 1.26 | 1.30 | 1.37 |
| Quinoline | 98.82 | 99.77 | 98.67 | 98.49 | 98.3 | 97.99 |

Table 7 below reports the area % data over time measured for solutions held in ethanol.

TABLE 7

| | t0 | 24 h | 48 h | 4 d | 7 d | 14 d |
|---|---|---|---|---|---|---|
| RT 1.4 min (Compound 4) | — | — | — | — | 0.03 | 0.05 |
| RT 3.2 min (Compound 2) | — | — | 0.06 | 0.07 | 0.09 | 0.12 |
| Quinoline Enantiomer | 0.54 | 0.80 | 0.96 | 1.24 | 1.51 | 1.87 |
| Quinoline | 99.46 | 99.20 | 98.98 | 98.70 | 98.37 | 97.96 |

Table 8 below reports the area % data over time measured for solutions held in acetonitrile. Epimerisation, but not chemical degradation, of the carboxamide was observed in acetonitrile.

TABLE 8

| | t0 | 3 d | 10 d |
|---|---|---|---|
| Quinoline Enantiomer | 0.87 | 1.34 | 1.73 |
| Quinoline | 99.13 | 98.66 | 98.27 |

### Example 8: Solubility Studies

### A. Form B Solubility Study

A solubility study was conducted for Form B. Small aliquots of each solvent tested were added at ambient temperature to an accurately weighed sample of Form B (˜20 mg). The aliquot volumes were typically 20-200 μL, up to a total volume of 1.0 mL. Complete dissolution of the Form B was determined by visual inspection. For solvents with solubility less than 20 mg/mL, the experiment was repeated using about 2 mg of Form B.

The estimated solubility in each solvent tested was based on the total solvent used to provide complete dissolution. It should be noted that the actual solubility may vary to some extent from the estimated (i.e., visually detected) solubility due to use of solvent aliquots that were too large, a slow rate of dissolution, or other factors. The estimated solubilities are reported in Table 9 below.

TABLE 9

| | SOLUBILITY |
|---|---|
| | AT 20° C. |
| SOLVENT | (mg/mL) |
| acetic acid | >430 |
| acetone | 20.3-22.6 |
| acetonitrile | 20.6-22.9 |
| anisole | <2 |
| butyl acetate | <2 |
| tert-butanol | <2 |
| chloroform | 30.9-36 |
| cyclohexane | <2 |
| dichloromethane | 36.2-43.4 |
| diisopropyl ether | <2 |
| dimethyl acetamide | 426-852 |
| dimethyl formamide | 402-804 |
| dimethyl sulfoxide | 424-848 |
| dioxane | 26.6-30.4 |
| ethanol | 2.1-2.6 |
| ethyl acetate | <2 |
| heptane | <2 |
| isopropyl acetate | <2 |
| methanol | 9.3-11.2 |
| methyl ethyl ketone | 9.3-14 |
| methyl isobutyl ketone | <2 |
| 2-methyl-tetrahydrofuran | <2 |
| methyl tert-butyl ether | <2 |
| N-methyl pyrrolidone | 424-848 |
| 1-propanol | <2 |
| isopropanol | <2 |
| tetrahydrofuran | 24.4-27.5 |
| toluene | <2 |
| water | <2 |

A suitable solvent for cooling crystallization should have high solubility for the solute as well as high potential recovery, i.e., the solvent generally should have high solubility for the solute at a high temperature and relatively low solubility for the solute at a low temperature (i.e., high temperature coefficient of solubility). With respect to their solubility at a low temperature, solvents suitable for cooling crystallization generally will have a solubility at 20° C. in the range of about 5 mg/mL to about 20 mg/mL.

### B. FaSSIF Blank Dissolution Media (Form A and Form B)

Solubility of Form A and Form B in phosphate buffer, pH 6.5 (FaSSIF Blank), dissolution media was determined after equilibrating the crystalline form in media at 25° C. for 1 day. High-performance liquid chromatography (HPLC) was used to determine the concentration in filtered solution. The solid residue was analyzed by PXRD to confirm the crystalline form retained. Solubilities measured for Form A and Form B were 0.50 mg/ml and 0.30 mg/ml, respectively.

### C. Acetonitrile Solubility Curve

A Crystal 16 instrument is used to generate the solubility curve with increasing temperature in acetonitrile. At least three accurately measured samples of (R)—N-(2-(4-cyanothiazolidin-3-yl)-2-oxoethyl)-6-morpholinoquinoline-4-carboxamide Form B are charged to Crystal 16 vials, acetonitrile (1.0 mL) is charged via a pipette, and the contents are agitated at 700 rpm. The resulting slurries are heated from 25 to 70° C. at a constant rate of 0.075° C./minute and the clear points for each concentration are determined by measuring turbidity.

### D. Acetonitrile and Methanol Solubility Curves

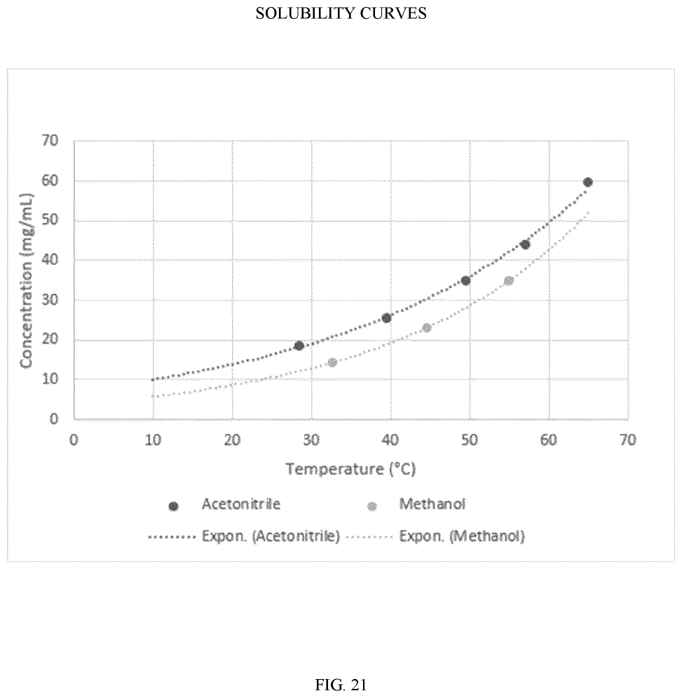

Form B solubility in acetonitrile and methanol with increasing temperature was evaluated. (R)—N-(2-(4-cyanothiazolidin-3-yl)-2-oxoethyl)-6-morpholinoquinoline-4-carboxamide Form B (each sample between 14.35 mg and 74.1 mg) and solvent (1.0 mL, either acetonitrile or methanol) were charged to a Crystal 16 vial. The contents were agitated at 700 rpm and the resulting slurries were heated from 25° C. to 70° C. at a constant rate of 0.075° C./minute. The clear points for each concentration of Form B were determined by measuring turbidity. The solubility curves generated based on the measured data are shown in FIG. 21.

### Example 9: Amorphous (R)—N-(2-(4-Cyanothiazolidin-3-yl)-2-oxoethyl)-6-morpholinoquinoline-4-carboxamide

### A. Preparation of Amorphous (R)—N-(2-(4-Cyanothiazolidin-3-yl)-2-oxoethyl)-6-morpholinoquinoline-4-carboxamide

Amorphous (R)—N-(2-(4-cyanothiazolidin-3-yl)-2-oxoethyl)-6-morpholino-quinoline-4-carboxamide can be isolated by various methods including freeze drying, anti-solvent precipitation, flash evaporation, melt quench, crash precipitation, and vapour diffusion. Several of these methods are illustrated below.

### (i) Flash Evaporation

(R)—N-(2-(4-cyanothiazolidin-3-yl)-2-oxoethyl)-6-morpholinoquinoline-4-carboxamide (25-30 mg) is dissolved in hot acetone, chloroform, tetrahydrofuran, or dichloromethane (400-700 μL). The solution is filtered through a 0.2 μm filter directly into a heated vial on a hotplate at about 120° C. and the solvent evaporated to yield a glassy solid. The glass residue generated in situ is used without further manipulation.

### (ii) Crash Precipitation

A saturated solution of (R)—N-(2-(4-cyanothiazolidin-3-yl)-2-oxoethyl)-6-morpholinoquinoline-4-carboxamide is prepared in selected solvents and filtered through a 0.2 μm PTFE filter directly into a round-bottomed flask containing antisolvent (about 10 volumes) at ambient temperature.

(iii) Freeze Drying

A solution of (R)—N-(2-(4-cyanothiazolidin-3-yl)-2-oxoethyl)-6-morpholinoquinoline-4-carboxamide in acetonitrile/water 1:1 is frozen in liquid nitrogen and dried in a freeze dryer (Christ Alpha 2-4 LDplus) overnight.

### (iv) Melt Quench

(R)—N-(2-(4-cyanothiazolidin-3-yl)-2-oxoethyl)-6-morpholinoquinoline-4-carboxamide (20-100 mg) is added to a HPLC vial and flushed with nitrogen gas. The vial is heated to 200° C. for five minutes and then quickly immersed in an ice/water bath to form amorphous material.

B. Physical Characterization of Amorphous (R)—N-(2-(4-Cyanothiazolidin-3-yl)-2-oxoethyl)-6-morpholinoquinoline-4-carboxamide

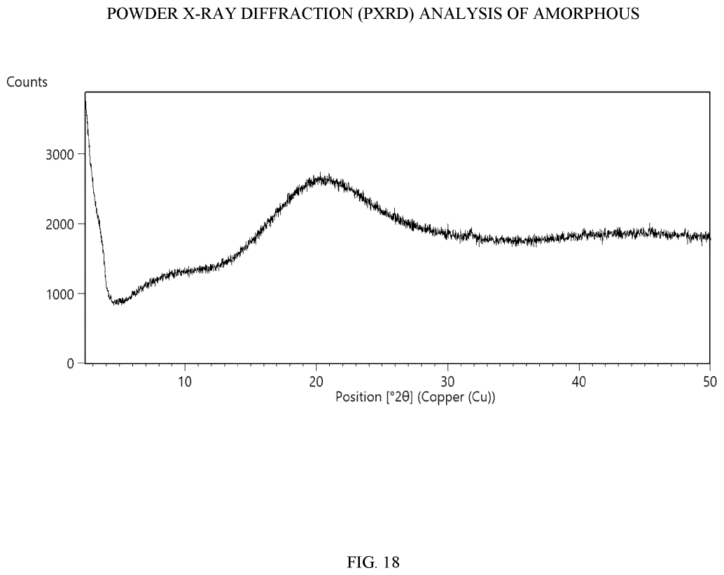

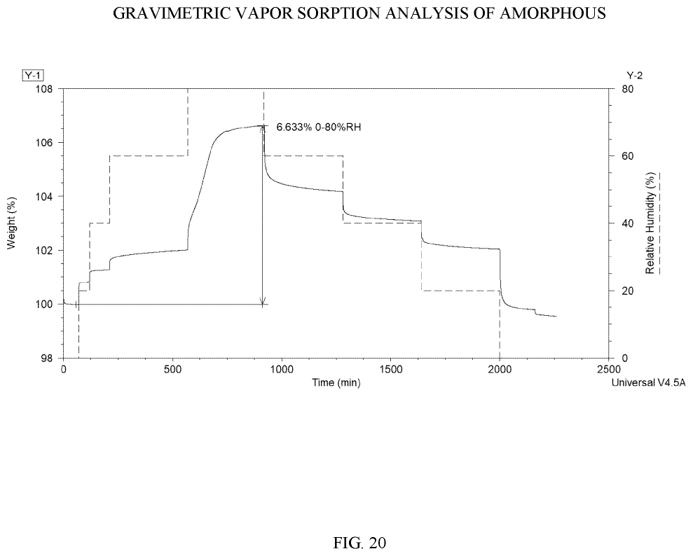

Amorphous (R)—N-(2-(4-cyanothiazolidin-3-yl)-2-oxoethyl)-6-morpholino-quinoline-4-carboxamide was characterized by PXRD (FIG. 18), differential scanning calorimetry (DSC) (FIG. 19), and gravimetric vapor sorption (GVS) (FIG. 20). The PXRD pattern of FIG. 18 shows that the amorphous compound has no regular crystalline order.

### Example 10: FAP Inhibition and Binding Assays

The hFAP protein used in the Examples was either commercially sourced or produced in insect cells as recombinant hFAP (Gp67-6HN-TEV-FAP (M39-A757), MW 89086.7 Da, or cd33-FAP (27-757)-6His, MW85926 Da). Recombinant hFAP protein was secreted from Sf21 cells in media, purified with affinity (batchmode, Ni excel resin, ÄKTA, GE Healthcare) and size exclusion chromatography (Superdex200, ÄKTA, GE Healthcare), concentrated to 19.5 mg/mL, snapfrozen in liquid nitrogen and stored in −80° C.

A. hFAP Inhibition Assay

(R)—N-(2-(4-Cyanothiazolidin-3-yl)-2-oxoethyl)-6-morpholinoquinoline-4-carboxamide was tested in a biochemical inhibition assay using hFAP enzyme at 0.24 nM FAC (Proteros, 38-760 (PR-0071)) and the substrate Ala-Pro-AMC (ARI-3144) at 20 μM FAC. 384 low volume black plates (Greiner #784076) were used. 4 μL, 0.48 nM enzyme solution (100 mM Tris HCl, 100 mM NaCl, 0.05% Chaps, pH 7.4) was added to 40 nL compounds (in DMSO) at 10 CR, 3-fold dilution series from 50 μM FAC. Plates were incubated for 15 min at rt in dark. 4 μL, 40 μM substrate solution (100 mM Tris HCl, 100 mM NaCl, 0.05% Chaps, pH 7.4) was added to each well. Plates were centrifuged at 1000 rpm and incubated for 30 min at rt in dark. The plates were read on a PHERAstar® reader with excitation 340 nm and emission 460 nm. Data were analyzed in Genedata Screener®. IC₅₀ values were determined by plotting % inhibition versus log compound concentration and using a one site dose response model. Raw data signals were normalized using 0.5% DMSO as 0% control and Reference Compound A (i.e., (S)—N-(2-(2-cyano-4,4-difluoropyrrolidin-1-yl)-2-oxoethyl) quinoline-4-carboxamide as reported in J. Med. Chem. 2014, 57, 3053) at 50 μM as 100% inhibitor control. Data are reported in Table 10.

B. hFAP Inhibition Assay (Tight Binders)

(R)—N-(2-(4-Cyanothiazolidin-3-yl)-2-oxoethyl)-6-morpholinoquinoline-4-carboxamide was tested in a biochemical inhibition assay using human Fibroblast activation protein alpha (hFAP) enzyme at 2.4 pM FAC (Proteros, 38-760 (PR-0071) and the substrate Ala-Pro-AMC (ARI-3144) at 20 μM FAC. 384 low volume black plates (Greiner #784076) were used. 4 μL, 4.8 pM enzyme solution (100 mM Tris HCl, 100 mM NaCl, 0.05% Chaps, pH 7.4) was added to 40 nL compounds (in DMSO) at 10 CR, 3-fold dilution series from 50 nM FAC. Plates were incubated for 15 min at rt in dark. 4 μL, 40 μM substrate solution (100 mM Tris HCl, 100 mM NaCl, 0.05% Chaps, pH 7.4) was added to each well. Plates were centrifuged at 1000 rpm and incubated for 2.5 h at rt in dark. The plates were read on a PHER Astar® reader with excitation 340 nm and emission 460 nm. Data were analyzed in Genedata Screener®. IC₅₀ values were determined by plotting % inhibition versus log compound concentration and using a one site dose response model. Raw data signals were normalized using 0.5% DMSO as 0% control and Reference Compound A (i.e., (S)—N-(2-(2-cyano-4,4-difluoropyrrolidin-1-yl)-2-oxoethyl) quinoline-4-carboxamide as reported in J. Med. Chem. 2014, 57, 3053) at 50 μM as 100% inhibitor control. Data are reported in Table 10.

C. hFAP Binding Assay

(R)—N-(2-(4-Cyanothiazolidin-3-yl)-2-oxoethyl)-6-morpholinoquinoline-4-carboxamide was tested in a direct binding assay using 8K surface plasmon resonance biosensor (GE Healthcare) at 20° C. Immobilization of hFAP (M39-A757) on a CMD200M sensor chip (Xantec) was performed using standard amine coupling procedure in immobilization buffer (10 mM HEPES, 150 mM NaCl, 0.05% Tween20, pH 7.4). The surface was washed with 10 mM NaOH, IM NaCl before being activated with EDC/NHS (GE Healthcare), followed by immobilization of hFAP (in 10 mM Acetate pH 5.0). Finally, the surface was deactivated by ethanolamine. Immobilization levels of hFAP were around 4000-6000 RU. The reference spot was treated as described, omitting the injection of hFAP. Compound concentration series were injected over the immobilized protein in increasing concentrations (2-500 nM) using single cycle kinetics in running buffer (20 mM TRIS, 150 mM NaCl, 0.05% Tween20, 1% DMSO, pH 7.4). Interaction models were fitted globally to the experimental traces, enabling determination of kₒₙ, k_{off} and K_{d}. Data are reported in Table 10.

TABLE 10

| ASSAY | RESULT |
|---|---|
| hFAP inhibition assay¹ | IC₅₀ = 0.37 nM |
| hFAP inhibition assay (Tight Binders)² | IC₅₀ = 0.084 nM |
| hFAP Binding assay³ | K_{d} = 0.012 nM |
| hFAP Binding assay³ | k₍ₒₙ₎ = 4900000 M⁻¹s⁻¹ |
| hFAP Binding assay³ | k_{(off)} = 0.000046 1/s³ |

¹IC₅₀ is reported after single measurement (n = 1) or as geometric mean for multiple measurements (n = 2-3).
²IC₅₀ is reported after single measurement (n = 1) or as geometric mean for multiple measurements (n = 2-6).
³K_{d} is reported after single measurement (n = 1) or as geometric mean for multiple measurements (n = 2-4). k₍ₒₙ₎ and k_{(off)} are reported after single measurement (n = 1) or as an average for multiple measurements (n = 2-4). NV is not valid.

### D. FAP Plasma Inhibition Assay

Plasma (anticoagulant K2EDTA) was used as the enzyme source: Human plasma (Pooled from AZ Biobank), Mouse plasma (AZ AST Biobank), and Cynomolgus plasma (BioIVT, #NHP00PLK2FNN, lot CYN222895). 384-Well black fluotrack PS plates (Greiner 781076) were used. 20 μL diluted plasma (Cynomolgus and Human plasma dilution 1:40, Mouse plasma dilution 1:67) in buffer (PBS, 0.1% BSA) was added to 0.6 μL compounds (in DMSO). Compounds were tested using 10 CR, 3-fold dilution series from 500 nM FAC. Two replicates for each assay point were run on the same plate. A fluorescence blank read was taken before substrate addition. Substrate, Ala-Pro-AMC (ARI-3144) stock solution (20 mM in DMSO) was diluted in buffer (PBS, 0.1% BSA) to 150 μM concentration and 20 μL added giving 75 μM FAC. Plates were incubated for 40 min at rt in the dark. The plates were read on a Beckman Paradigm® reader with excitation 360 nm and emission 465 nm. Data were analyzed in Excel (IDBS XLfit Add-In) using a one site dose response model (4-parameter logistic fit). IC₅₀ values were determined by plotting % inhibition versus log compound concentration. Raw data signals were normalized using 1.5% DMSO in diluted plasma as 0% control and 1.5% DMSO in buffer (no plasma) as 100% inhibitor control. Data are reported in Table 11.

TABLE 11

| ASSAY | IC₅₀ (nM) |
|---|---|
| FAP Human Plasma¹ | 0.36 |
| FAP Mouse Plasma² | 0.52 |
| FAP Cyno Plasma³ | 0.40 |

¹IC₅₀ is reported after single measurement (n = 1) or as geometric mean for multiple measurements (n = 4).
²IC₅₀ is reported after single measurement (n = 1) or as geometric mean for multiple measurements (n = 4).
³IC₅₀ is reported after single measurement (n = 1).

### Example 11: hPrep Inhibition Assay

(R)—N-(2-(4-Cyanothiazolidin-3-yl)-2-oxoethyl)-6-morpholinoquinoline-4-carboxamide was tested in a biochemical inhibition assay using Prolyl endopeptidase, Prolyl Oligopeptidase (hPREP) enzyme at 0.6 nM FAC (R&D Systems, 4308-SE) and the substrate Z-Gly-Pro-amino-methylcoumarin (Bachem, I-1145) at 50 μM FAC. 384 Low volume black plates (Greiner #784076) were used. 4 μL, 1.2 nM enzyme solution (25 mM Tris HCl, 250 mM NaCl, 0.01% Triton X-100, 5 mM Glutathione, pH 7.5) was added to 40 nL compounds (in DMSO) at 10 CR, 3-fold dilution series from 50 μM FAC. Plates were incubated for 15 min at rt in dark. 4 μL, 100 μM substrate solution (25 mM Tris HCl, 250 mM NaCl, 0.01% Triton X-100, 5 mM Glutathione, pH 7.5) was added to each well. Plates were centrifuged at 1000 rpm and incubated for 20 min at rt in dark. The plates were read on a PHERAstar® reader with excitation 340 nm and emission 460 nm. Data were analyzed in Genedata Screener®. IC₅₀ values were determined by plotting % inhibition versus log compound concentration and using a one site dose response model. Raw data signals were normalized using 0.5% DMSO as 0% control and Reference Compound B (i.e., (R)—N-(2-(4-cyanothiazolidin-3-yl)-2-oxoethyl)-7-methylquinoline-4-carboxamide) at 50 μM as 100% inhibitor control. Data are reported in Table 12.

TABLE 12

| | PREP |
|---|---|
| COMPOUND | IC₅₀ (nM)¹ |
| (R)-N-(2-(4-Cyanothiazolidin-3-yl)-2-oxoethyl)- | 1200 |
| 6-morpholinoquinoline-4-carboxamide | |
| B² | 5.2 |
| C³ | 7.1 |

¹IC₅₀ is reported after single measurement (n = 1) or as geometric mean for multiple measurements (n = 2-9).
²Reference Compound B: (R)-N-(2-(4-cyanothiazolidin-3-yl)-2-oxoethyl)-7-methylquinoline-4-carboxamide.
³Reference Compound C: (R)-N-(2-(4-cyanothiazolidin-3-yl)-2-oxoethyl)-quinoline-4-carboxamide.

### Example 12: hDPP Inhibition Assays

A. hDPP7 Inhibition Assay

(R)—N-(2-(4-Cyanothiazolidin-3-yl)-2-oxoethyl)-6-morpholinoquinoline-4-carboxamide was tested in a biochemical inhibition assay using human dipeptidylpeptidase 7 (hDPP7) enzyme at 15 nM FAC (BPS Bioscience, #80070) and the substrate Ala-Pro-amino-methylcoumarin (BPS Bioscience, #80305) at 5 μM FAC. The enzymatic reactions were conducted in duplicate at rt for 30 min in 50 μL DPP assay buffer (BPS Bioscience, #80300). Compound solutions (in DMSO) at 10 CR, 3-fold dilution series were prepared in assay buffer ten-fold higher than the final concentration, and 5 μL of the dilution was added to a 50 μL reaction so that the highest compound concentration was 100 μM FAC and the concentration of DMSO was 1% in all wells. The plates were read on a Tecan Infinite M1000 microplate reader with excitation 340 nm and emission 460 nm. Data were analyzed in Graph Pad Prism. IC₅₀ values were determined by plotting % inhibition versus log compound concentration and using a one site dose response model. Raw data signals were normalized using 1% DMSO as 0% control and no enzyme as 100% inhibitor control. Data are reported in Table 13.

B. hDPP8 Inhibition Assay

(R)—N-(2-(4-Cyanothiazolidin-3-yl)-2-oxoethyl)-6-morpholinoquinoline-4-carboxamide was tested in a biochemical inhibition assay using human dipeptidylpeptidase 8 (hDPP8) enzyme at 1.5 nM FAC (BPS Bioscience, #80080) and the substrate Ala-Pro-amino-methylcoumarin (BPS Bioscience #80305) at 5 μM FAC. The enzymatic reactions were conducted in duplicate at rt for 30 min in 50 μL DPP assay buffer (BPS Bioscience, #80300). Compound solutions (in DMSO) at 10 CR, 3-fold dilution series were prepared in assay buffer ten-fold higher than the final concentration, and 5 μL of the dilution was added to a 50 μL reaction so that the highest compound concentration was 100 μM FAC and the concentration of DMSO was 1% in all wells. The plates were read on a Tecan Infinite M1000 microplate reader with excitation 340 nm and emission 460 nm. Data were analyzed in Graph Pad Prism. IC₅₀ values were determined by plotting % inhibition versus log compound concentration and using a one site dose response model. Raw data signals were normalized using 1% DMSO as 0% control and no enzyme as 100% inhibitor control. Data are reported in Table 13.

C. hDPP9 Inhibition Assay

(R)—N-(2-(4-Cyanothiazolidin-3-yl)-2-oxoethyl)-6-morpholinoquinoline-4-carboxamide was tested in a biochemical inhibition assay using human dipeptidylpeptidase 9 (hDPP9) enzyme at 0.4 nM FAC (BPS Bioscience, #80090) and the substrate Ala-Pro-amino-methylcoumarin (BPS Bioscience #80305) at 5 μM FAC. The enzymatic reactions were conducted in duplicate at rt for 30 min in 50 μL DPP assay buffer (BPS Bioscience, #80300). Compound solutions (in DMSO) at 10 CR, 3-fold dilution series were prepared in assay buffer ten-fold higher than the final concentration, and 5 μL of the dilution was added to a 50 μL reaction so that the highest compound concentration was 100 μM FAC and the concentration of DMSO was 1% in all wells. The plates were read on a Tecan Infinite M1000 microplate reader with excitation 340 nm and emission 460 nm. Data were analyzed in Graph Pad Prism. IC₅₀ values were determined by plotting % inhibition versus log compound concentration and using a one site dose response model. Raw data signals were normalized using 1% DMSO as 0% control and no enzyme as 100% inhibitor control. Data are reported in Table 13.

TABLE 13

| ASSAY | IC₅₀ (nM)¹ |
|---|---|
| hDPP7 | 33000 |
| hDPP8 | 13000 |
| hDPP9 | 1900 |

¹IC₅₀ is reported after single measurement (n = 1).

Although specific embodiments and examples have been described above, these embodiments and examples are only illustrative and do not limit the scope of the disclosure. Changes and modifications can be made in accordance with ordinary skill in the art without departing from the disclosure in its broader aspects as defined in the following claims. For example, any embodiment described herein can be combined with any other suitable embodiment described herein to provide additional embodiments.

What is claimed is:

1. A crystalline form of N-(2-(4-cyanothiazolidin-3-yl)-2-oxoethyl)-6-morpholinoquinoline-4-carboxamide.

2. The crystalline form of claim 1 that is a crystalline form of (R)—N-(2-(4-cyanothiazolidin-3-yl)-2-oxoethyl)-6-morpholinoquinoline-4-carboxamide.

3. The crystalline form of claim 2 characterized by a transmission X-ray powder diffraction pattern comprising peaks at 18.7±0.2° 2θ and 22.4° 2θ±0.2° 2θ.

4. The crystalline form of claim 3, wherein the transmission X-ray powder diffraction pattern further comprises at least one peak selected from the group consisting of 12.0±0.2° 2θ, 18.4±0.2° 2θ, 19.8±0.2° 2θ, and 21.7° 2θ±0.2° 2θ.

5. The crystalline form of any of claims 2 to 4 further characterized by a solid-state ¹³C NMR spectrum comprising at least one peak selected from the group consisting of 166.9±0.2 ppm, 130.2±0.2 ppm, 118.6±0.2 ppm, 117.4±0.2 ppm, 110.0±0.2 ppm, 49.9±0.2 ppm, 46.6±0.2 ppm, and 34.5±0.2 ppm.

6. The crystalline form of claim 5, wherein the solid-state ¹³C NMR spectrum comprises peaks at 49.9±0.2 ppm and 46.6±0.2 ppm.

7. The crystalline form of any of claims 2 to 6 further characterized by a differential scanning calorimetry curve comprising a melting endotherm having an onset between about 185° C. to about 200° C.

8. The crystalline form of any of claims 3 to 7 further characterized by a thermogravimetric analysis thermogram wherein the crystalline form exhibits a weight loss of less than about 0.5 weight % from about 25° C. to about 110° C.

9. The crystalline form of any of claims 3 to 8 further characterized by a gravimetric vapor sorption plot wherein the crystalline form exhibits a reversible moisture uptake of less than about 0.5 weight % from about 0% relative humidity to about 80% relative humidity at 25° C.±0.1° C.

10. The crystalline form of any of claims 3 to 9, wherein the crystalline form is a crystalline anhydrate.

11. The crystalline form of claim 2, wherein:
the transmission X-ray powder diffraction pattern comprises at least one peak selected from the group consisting of 12.0±0.2° 2θ, 18.4±0.2° 2θ, 19.8±0.2° 2θ, and 21.7°2θ±0.2°2θ; and
the transmission X-ray powder diffraction pattern does not comprise peaks at 18.7±0.2° 2θ and 22.4° 2θ±0.2° 2θ having a medium or stronger relative intensity.

12. The crystalline form of claim 11, wherein the transmission X-ray powder diffraction pattern further comprises at least two peaks selected from the group consisting of 12.0±0.2° 2θ, 18.4±0.2° 2θ, 19.8±0.2° 2θ, and 21.7°2θ±0.2° 2θ.

13. The crystalline form of any of claim 2, 11, or 12 further characterized by a solid-state ¹³C NMR spectrum comprising at least one peak selected from the group consisting of 166.1±0.2 ppm, 130.7±0.2 ppm, 117.9±0.2 ppm, 108.6±0.2 ppm, and 35.2±0.2 ppm.

14. The crystalline form of claim 13, wherein the solid-state ¹³C NMR spectrum comprises peaks at 166.1±0.2 ppm, 117.9±0.2 ppm, and 108.6±0.2 ppm.

15. The crystalline form of claim 2 or any of claims 11 to 14 further characterized by a differential scanning calorimetry curve comprising a melting endotherm having an onset temperature between about 165° C. to about 180° C.

16. The crystalline form of any of claims 11 to 15 further characterized by a thermogravimetric analysis thermogram wherein the crystalline form exhibits a weight loss of less than about 0.5 weight % from about 25° C. to about 110° C.

17. The crystalline form of any of claims 11 to 16 further characterized by a gravimetric vapor sorption plot wherein the crystalline form exhibits a reversible moisture uptake of less than about 0.5 weight % from about 0% relative humidity to about 80% relative humidity at 25° C.±0.1° C.

18. The crystalline form of any of claims 11 to 17, wherein the crystalline form is a crystalline anhydrate.

19. The crystalline form of claim 1 that is a crystalline form of (R,S)—N-(2-(4-cyanothiazolidin-3-yl)-2-oxoethyl)-6-morpholinoquinoline-4-carboxamide.

20. A composition comprising at least two crystalline forms selected from the group consisting of:
the crystalline form of any of claims 3 to 10;
the crystalline form of any of claims 11 to 18; and
the crystalline form of claim 19.

21. A pharmaceutical composition comprising the crystalline form of any of claims 1 to 19, and one or more pharmaceutically acceptable excipients.

22. A method of treating or preventing an FAP-mediated condition in a subject suffering from or susceptible to the FAP-mediated condition, the method comprising administering to the subject a therapeutically effective amount of a crystalline form of any of claims 1 to 19.
